SEQUENCE LISTING (19 sequences)

SEQ ID NO: 1 - DNA, 501 bp, Staphylococcus aureus
atgcataagttagcacatacaagttttgggattgttgggatgtttgttaatacttgtatagtagctaaatatgtgattattaattgggagatgtttagcatgaaaaaagtaaataacgacactgtatttggaattttgcaattagaaacacttttgggtgacattaactcaattttcagcgagattgaaagcgaatacaaaatgtctagagaagaaattttaattttactaactttatggcaaaaaggttttatgacgcttaaagaaatggacagatttgttgaagttaaaccgtataagcgtacgagaacgtataataatttagttgaattagaatggatttacaaagagcgtcctgttgacgatgaaagaacagttattattcatttcaatgaaaagttacaacaagagaaagtagagttgttgaatttcatcagtgatgcgattgcaagtagagcaacagcaatgcaaaatagtttaaacgcaattattgctgtgtaa

SEQ ID NO: 2 - protein, 166 aa, Staphylococcus aureus
MHKLAHTSFGIVGMFVNTCIVAKYVIINWEMFSMKKVNNDTVFGILQLETLLGDINSIFSEIESEYKMSREEILILLTLWQKGFMTLKEMDRFVEVKPYKRTRTYNNLVELEWIYKERPVDDERTVIIHFNEKLQQEKVELLNFISDAIASRATAMQNSLNAIIAV

SEQ ID NO: 3 - DNA, 750 bp, Staphylococcus aureus
gtggactatcaaactttcgaaaaggtcaataaattcataaatgtaaaagcgtacatattttttctcactcaagagttaaagcaacaatataaattatcattaaaagaattgttgatattagcatatttttattacaaaaatgaacacagtatttcactaaaagaaatcattggtgacatactttacaaacaatctgatgttgtaaagaacattaagtcactatctaaaaaaggatttataaataagtctagaaacgaagcagatgaacgccgtatttttgtttcagttactccaatacaacgtaaaaagattgcttgtgttattaatgagttagataaaataattaaaggatttaataaggaaagagactacataaaatatcaatgggctccaaaatatagcaaagaattttttatactttttatgaacattatgtactcaaaagattttttaaaatatcgatttaatttaacatttcttgatttatctatcttatatgtaatatacatctcgaaaaaatgagatactaaatttaaaagatttgtttgaaagtattagatttatgtatcctcaaattgttaggtcagttaatagattaaataataaaggtatgctaatcaaagaacgatcccttgcagatgaaaggattgtgttaatcaaaataaataaaatacaatataacactattaaaagcatattcacagatacttccaagattctcaaaccaagaaaatttttcttttaaattta

SEQ ID NO: 4 - protein, 247 aa, Staphylococcus aureus
MDYQTFEKVNKFINVKAYIFFLTQELKQQYKLSLKELLILAYFYYKNEHSISLKEIIGDILYKQSDVVKNIKSLSKKGFINKSRNEADERRIFVSVTPIQRKKIACVINELDKIIKGFNKERDYIKYQWAPKYSKEFFILFMNIMYSKDFLKYRFNLTFLDLSILYVISSRKNEILNLKDLFESIRFMYPQIVRSVNRLNNKGMLIKERSLADERIVLIKINKIQYNTIKSIFTDTSKILKPRKFFF

SEQ ID NO: 5 - DNA, 1307 bp, Staphylococcus aureus
cagtagatgctcatctttttttagaacttttttaaggttgaaaatgtatatcacattttatacacatttgatttgtaagaaatgttttgatttatacaaatcatatcttgaaaaataaccaatttagcctcattcggtttgatttaatttgttaaatttaaggctaactaattaatttagtttaatcaattttcatgagagttatatgtaataaaaattcaatgcgtatcttttttgaagaaatatatgtagaattgttgcaatttaatggtaatattgatatattttctttgtatataaattataaaattaatatgtaatagagtgatttgttttatgtactattatcttatttctaaatattaactctattgattattggtttttatacttatttaattttattcaactttgacaattgaatagaaagcaagtttatttacacttgtagttttatgcataagttagcacatacaagttttggattgttgggatgtttgttaatacttgtatagtagctaaatatgtgattattaattgggagatgtttagcatgaaaaaagtaaataacgacactgtatttggaattttgcaattagaaacacttttgggtgacattaactcaattttcagcgagattgaaagcgaatacaaaatgtctagagaagaaattttaattttactaactttatggcaaaaaggttttatgacgcttaaagaaatggacagatttgttgaagttaaaccgtataagcgtacgagaacgtataataatttagttgaattagaatggatttacaaagagcgtcctgttgacgatgaaagaacagttattattcatttcaatgaaaagttacaacaagagaaagtagagttgttgaatttcatcagtgatgcgattgcaagtagagcaacagcaatgcaaaatagtttaaacgcaattattgctgtgtaagtttaatagcataaaaagaggttttcattaagttgaaaacctctttttgttgttggcattaatttttcaaatgttgactactcaatcctaaattataaatagtatagcgcagcaaatgcttaagaaattttttctatggcacaaatgaatggagcatgattacgttggtttaaaaattgatattgcaaaacttgcgcatgcttttgatccaaagtactcaagtaatcaagcaatgcatgcttctcaatttgtccttcgctatgaccatgatatataacaagtacaataataccttcaattgacattaatgatagcaatgaattaatagcttggattgtcgtgtcaggctttg

SEQ ID NO: 6 - DNA, 33 bp, Staphylococcus aureus
ggaagcttaaacatcatttctgaagttatcggc

SEQ ID NO: 7 - DNA, 34 bp, Staphylococcus aureus
gggactagtgaaggatgatgaaaatgaaaacacg

SEQ ID NO: 8 - DNA, 23 bp, Staphylococcus aureus
gagcatatccacttttcttggag

SEQ ID NO: 9 - DNA, 22 bp, Staphylococcus aureus
cacaatagagagatgtcacgtc

SEQ ID NO: 10 - DNA, 21 bp, Staphylococcus aureus
caaagcctgacacgacaatcc

SEQ ID NO: 11 - DNA, 22 bp, Staphylococcus aureus
ctgaaagatgagacagtagatg

SEQ ID NO: 12 - DNA, 827 bp, Staphylococcus aureus
aattcaatgcgtatcttttttgaagaaatatatgtagaattgttgcaatttaatggtaatattgatatattttctttgtatataaattataaaattaatatgtaatagagtgatttgttttatgtactattatcttatttctaaatattaactctattgattattggtttttatacttatttaattttattcaactttgacaattgaatagaaagcaagtttatttacacttgtagttttatgcataagttagcacatacaagttttgggattgttgggatgtttgttaatacttgtatagtagctaaatatgtgattattaattgggagatgtttagcatgaaaaaagtaaataacgacactgtatttggaattttgcaattagaaacacttttgggtgacattaactcaattttcagcgagattgaaagcgaatacaaaatgtctagagaagaaattttaattttactaactttatggcaaaaaggttttatgacgcttaaagaaatggacagatttgttgaagttaaaccgtataagcgtacgagaacgtataataatttagttgaattagaatggatttacaaagagcgtcctgttgacgatgaaagaacagttattattcatttcaatgaaaagttacaacaagagaaagtagagttgttgaatttcatcagtgatgcgattgcaagtagagcaacagcaatgcaaaatagtttaaacgcaattattgctgtgtaagtttaatagcataaaaagaggttttcattaagttgaaaacctctttttgttgttggcattaatttttcaaatgttgactactcaat

SEQ ID NO: 13 - DNA, 27 bp, Staphylococcus aureus
ggatccgcccatgaaactttccatctg

SEQ ID NO: 14 - DNA, 27 bp, Staphylococcus aureus
ggatccgcgaacgttatgacgttggag

SEQ ID NO: 15 - DNA, 25 bp, Staphylococcus aureus
ttcgaatcgtgcgctctcctgttcc

SEQ ID NO: 16 - DNA, 26 bp, v
ttcgaatggcttattggcatcctggc

SEQ ID NO: 17 - DNA, 23 bp, Staphylococcus aureus
cctaaagttacagatgcaatacc

SEQ ID NO: 18 - DNA, 19 bp, Staphylococcus aureus
cgagggttagtcaaagttg

SEQ ID NO: 19 - DNA, 21 bp, Staphylococcus aureus
gtgccattgaaatcactcctt

(19) United States
(12) Patent Application Publication
(10) Pub. No.: US 2003/0171563 A1
(43) Pub. Date: Sep. 11, 2003

(54) Regulators of bacterial virulence factor expression

(76) Inventor: Peter J. McNamara, Madison, WI (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/145,602

(22) Filed: May 13, 2002

Related U.S. Application Data
(60) Provisional application No. 60/291,917, filed on May 18, 2001.

(51) Int. Cl. C07H 21/02; C07H 21/04

(52) U.S. Cl. 536/23.1

(57) ABSTRACT

Genetic loci that encode polypeptides that regulate the expression of virulence factors in bacteria, particularly S. aureus. Methods of detecting the gene or gene products for purposes of detecting S. aureus or diagnosing a patient suspected of being infected by S. aureus.

This application claims priority from a provisional patent application entitled “Regulators of Bacterial Virulence Factor Expression,” filed in the name Kimberly-Clark Worldwide, Inc. on May 18, 2001, and given patent application serial No. 60/291,917, which is hereby incorporated by reference in its entirety.

### BACKGROUND OF THE INVENTION

Staphylococcus aureus can cause many different diseases, including skin infections, pneumonia, endocarditis, and toxic shock syndrome. The virulence of S. aureus is dependent upon the organism's ability to elaborate cell surface proteins and extra-cellular toxins and enzymes. These toxins include hemolysins, proteases, enterotoxins, and toxic shock syndrome toxin.

Virulence factor regulation. The expression of many virulence factors of S. aureus is controlled by the products of several loci. In laboratory cultures, virulence factor expression is altered as the bacteria transition from the exponential phase to stationary phase of growth (Abbas-Ali and Colman, 1997; Ji et al., 1995; Novick et al., 1990). Throughout the post-exponential phase of growth the components encoded by agr, sae, ar, and at least six MarR-family regulators (sarA and the Sar-homologues) function together to repress the transcription of genes encoding cell-surface virulence factors while increasing the transcription of genes encoding extracellular toxins and enzymes (Janzon and Arvidson, 1990; Novick et al., 1993). Measurements of the levels of translation products in strains with regulatory gene mutations have been complicated by the lack of quantitative, reproducible, specific assays. However, the reduction of exponential phase cell-surface proteins and the increase in all but one of the regulated post-exponential phase proteins that have been examined mirrors the pattern of their transcription (Bjorklind and Arvidson, 1980; Morfeldt et al., 1996; Novick et al, 1993).

A model of virulence factor regulation. The current understanding of the function of components encoded by agr, sae, ar, sarA, and the Sar-homologues, has lead to a model that describes the mechanism of virulence factor regulation. Central in the model is agr (FIG. 1, item 1) (Peng et al, 1988). This locus consists of divergent messages transcribed from adjacent promoters designated P2 and P3 (Janzon and Arvidson, 1990; Kornblum et al., 1990). The P2 promoter message, RNAII, encodes four proteins, AgrA, AgrB, AgrC, and AgrD (Kornblum et al., 1990; Norvick et al., 1995). These proteins are involved in a partially self-inducing, pheromone-sensing, signal transduction circuit. Two of the agr-encoded proteins share sequence homology with components of other bacterial signal transduction systems (Norvick et al., 1995). These proteins, AgrC and AgrA, act as a histidine-kinase sensor and a response regulator, respectively. The activating signal of the agr system is a peptide pheromone modified from the pre-peptide protein AgrD (Ji et al., 1995). AgrB is believed to be the enzyme responsible for the maturation (modification and secretion) of the peptide pheromone (Ji et al., 1997).

The agr system begins to function when AgrC binds the AgrD-derived peptide signal (Ji et al., 1997, Novick et al., 1995). Like other bacterial sensor proteins, the binding of the signal to the sensor protein initiates an autophosphorylation event and presumably, a concomitant activating conformational change to AgrC (Kornblum et al., 1990; Lina et al., 1998). The phosphate group on AgrC is thought to be transferred to the regulator protein, AgrA. This phosphate transfer results in an activating, again presumably conformational, change to AgrA. Unlike other bacterial signal transduction systems where the activated regulator protein directly initiates the transcription of target promoters, genetic evidence provides support that activated AgrA functions with the translation product of a genetically unlinked locus named sarA to up-regulate transcription from the agr promoters (Cheung et al., 1994; Cheung et al., 1995). The agr promoters are the only known targets of AgrA. The sarA product, SarA (FIG. 1, item 2), has been shown to bind DNA between the two agr promoters (Chien et al., 1998; Heinrichs et al., 1996; Rechtin et al., 1999). While the activation of agr has been genetically and biochemically examined, the specific roles of AgrA and SarA remain unclear. Regardless of the mechanism, the result of the increase in P2 and P3 transcription is an amplification of the activating circuit encoded by RNAII and the high-level production of a 514-ribonucleotide RNA known as RNAIII (Janzon and Arvidson, 1990; Morfeldt et al., 1996; Novick et al., 1993).

SarA is transcribed from three different overlapping messages. These messages, from largest to smallest, are known as “B”, “C”, and “A” (Bayer et al., 1996). All the messages are initiated from distinct upstream promoters (P2, P3, and P1, respectively) and end at a common terminator downstream of the SarA open reading frame. The Pi transcript is the dominant message in S. aureus 8325-4 derived laboratory strains. Transcription from the sarA P1 and P2 promoters is dependent on primary sigma factor in S. aureus (σ^{A}), while the sarA P3 promoter is dependent on the multiple-stress-responsive sigma factor (σ^{B}) (Deora et al., 1997; Palma and Cheung, 2001). Virulence factor regulation has been studied in S. aureus strains derived from 8325-4. These strains have a mutation in the rsbu-encoded phosphatase and phenotypically have a reduced stress response (Kullik et al., 1998). Recent experiments have shown that full σ^{B} activity enhances transcription of sarA, decreases transcription of agr, and modulates virulence factor gene transcription (Chan and Foster, 1998; Kullik et al., 1998).

In addition to encoding SarA, the three sar messages play a direct role in virulence factor regulation. For example, transcriptional attenuation of the gene encoding protein A (spa) requires the sarA “A” message in sar-minus strains, while the sarA “B” and “C” messages complement agr-minus strains (Bayer et al., 1996; Cheung and Projan, 1994). These data need to be viewed with caution. The DNA fragments encoding the sarA messages were cloned on multi-copy number plasmids. These constructs may be expected to alter the concentration of SarA which can have a profound effect on the levels, and therefore activity, of the other regulators involved in the control of virulence factor expression (see below). Furthermore, interpretation of the sarA complementation studies is difficult because dissimilar phenotypes have been reported in sarA-minus strains sharing the same genetic background (Chan and Foster, 1998; Chien et al., 1998).

The MarR-family and the SarA-homologues. Complexity is added to the model of virulence factor regulation by the recent identification of additional regulatory proteins that modulate virulence factor expression. These modulators are members of the MarR-family of bacterial regulators (ExPASy Prosite, PS01117). The MarR-family of regulators is named after a repressor of regulons involved in multiple antibiotic resistance and oxidative stress in E. coli (Cohen et al., 1993). This family also includes regulators of anabolic pathways, toxins, sporulation, and protease production (Ludwig et al., 1995; Ruppen et al., 1988; Thomson et al., 1997). The majority of MarR homologues are repressors; however, at least one family member appears to act as an activator (Thomson et al., 1997). Thirteen MarR-family members can be identified by BLASTP searches of the S. aureus (FIG. 11). The genes encoding these proteins are present in the genome databases of strains N315, Mu50, COL, and 8325-4 (Kuroda et al., 2001). Included in this group are SarA, SarR, SarS, SarT, SarU, and Rot. With regard to the regulation of toxin production, the remaining homologues have yet to be characterized in regard to their effect on toxin production. This includes TcaR (B. Berger-Bachi, personal communication). Even though the genes encoding these Sar-homologues are distributed non-uniformly around the genome, an operon naming system is utilized (Kuroda et al., 2001).

Rot, SarR, SarS, SarT, and SarU are all components of the network of regulators that includes RNAIII and SarA. Genetic and biochemical data strongly implicate Rot and SarS (FIG. 1, item 3) in the repression of toxin gene transcription and the activation of cell-surface gene transcription. While we identified rot by screening a pool of transposon mutants, SarS was isolated from lysates using target promoter DNA fragments linked to magnetic beads (McNamara et al., 2000; Tegmark et al., 2000). In vitro, SarS has been shown to bind the promoter region of hla (the gene encoding the α-toxin), sspA (the gene encoding the V8 protease), spa, and rnaIII (the gene encoding agr RNAIII), although an effect on transcription was only seen with hla and spa (Cheung et al., 2001; Tegmark et al., 2000). SarR (FIG. 1, item 4) was isolated using a DNA-column with sarA P2 promoter fragments (Marrack and Kappler, 1990). When compared to wild-type strains, sarR mutant strains show increased expression of SarA. SarT (FIG. 1, item 6) was identified in the sequence of the S. aureus strain COL genome by homology to SarA (Schmidt et al., 2001). SarT is required for the transcriptional repression of rnaIII and hla. Although not included in our model, as we described with rot, an element encoded by agr appears to down-regulate expression of SarT (McNamara et al., 2000; Schmidt et al., 2001). Transcription of hla, the only virulence factor gene examined to date in sarT-minus strains, is dependent upon the repression of SarT by SarA (Schmidt et al., 2001). SarU (FIG. 1, item 5) appears to be required for transcription of both agr and sarA.

It is clear that the Sar-homologues affect the transcription of other Sar-homologues genes as well as agr. As mentioned above, the levels of SarS and SarT are dependent on the level of SarA (Cheung et al., 2001; Schmidt et al., 2001) and the level of SarA is related to the level of SarR (Manna and Cheung, 2001). Preliminary data indicate that Rot is involved in the down-regulation of transcription of SarS. Therefore, increased expression of a Sar-homologue can influence the level of other Sar-homologues, regulators, and ultimately, virulence factor genes. As mentioned above, sarA when cloned on a multi-copy plasmid and moderately overexpressed in S. aureus both negatively affects bacterial growth and reduces transcription of genes that are normally positively regulated by SarA in wild-type strains (Tegmark et al., 2001).

DNA binding sites and the structure of the Sar homoloques. The DNA recognition sequence for recombinant SarA (rSarA) binding has been examined by several groups. DNase I footprinting and sequence analysis of sequences upstream of regulated genes defined specific “AT”-rich binding sites and have shown that rSarA binds as a dimer protecting between 20 to 38-bp of DNA depending on the report (Cheung and Projan, 1994; Rechtin et al., 1999; Tegmark et al., 2001). In one study, rSarA was shown to bind linear DNA with limited sequence specificity (Tegmark et al., 2001). Instead, DNA fragments with a minimum “AT” content of 76% were shown to be sufficient for rSarA-binding. In this same study, sequences with slightly higher binding affinities were also found. These sequences corresponded to the specific sequences that were reported by the other investigators (Tegmark et al., 2001). It is difficult to believe that SarA is a nonspecific DNA binding protein. As the archetype for the Sar-homologues, the data of Tegmark et al. would imply that all the SarA-homologues are nonspecific DNA binding proteins (Tegmark et al., 2001). This leads to the question of how mutations in different Sar-homologue genes confer different phenotypes. While the levels of the various Sar-homologues may play a role, SarA and the Sar-homologues may require supercoiled rather than linear DNA for specific binding.

Crystal structures were determined for a rSarA monomer and a monomeric rSarA-6mer DNA complex (Schumacher et al., 2001a). rSarA has four α-helices domains and two inducible regions that consist of a β-hairpin and a carboxy-terminal loop. Studies of the rSarA-DNA complex revealed that the inducible domains in rSarA undergo extensive conformational changes that result in the formation of extended α-helices which wrap around DNA having a D-DNA-like conformation. Caution is indicated in accepting these data because they were obtained for a monomeric form of rSarA bound to a short DNA sequence (Schumacher et al., 2001b). DNase I protection and gel shift assays demonstrate that SarA binds DNA as a dimer, protects at least 20-bp of DNA, and introduces bends into the target DNA (Rechtin et al., 1999; Tegmark et al., 2001).

Structural and binding properties have also been determined for recombinant SarR (rSarR). DNase I protection and gel shift assays have demonstrated that rSarR binds DNA surrounding all three sarA promoters, although a specific DNA binding sequences were not defined (Liu et al., 2001; Manna and Chueng, 2001). Like rSarA, rSarR was shown to bind to DNA as a dimer. The crystal structure studies revealed that rSarR has both a classic helix-turn-helix motif for DNA binding in the major groove and a loop region involved in recognition of the minor groove (Liu et al., 2001). rSarR was shown to interact with approximately 27 bp of target DNA and to induce bends within the target DNA (Liu et al., 2001; Manna and Chueng, 2001). It is reasonable to assume that the characterized Sar-homologues that are most closely related to SarR (SarA, Rot, and SarT) bind to DNA as dimers using the helix-turn-helix motif and loop region and act as DNA-bending proteins. It is unknown if SarR, Rot, and SarT can form heterodimers. In contrast, SarS and SarU have two DNA-binding domains and probably bind DNA as a monomer, although other higher ordered quaternary structures are possible.

Other two-component signal transductions systems in the regulatory network. Transposon mutagenesis of a wild-type strain of S. aureus coupled with a screen for altered extracellular protein production was used to identify sae (FIG. 1, item 7) (Giraudo et al., 1997; Giraudo et al., 1994; Rampone et al., 1996). The sae locus encodes a two-component signal transduction system that functions to stimulate transcription of the genes encoding α-toxin and β-toxin (hlb) and coagulase (Giraudo et al., 1994). Unlike agr-minus strains, a sae mutation results in strains with decreased transcription of the gene encoding protein A (spa). The coding capacity of the sae operon, activating stimuli of the sae sensor, mechanism of action of the response regulator, and role in the virulence factor gene regulatory network remain unknown. However, the relevance of sae to virulence has been verified in an intraperitoneal mouse model of infection (Rampone et al., 1996).

ArlRS encodes a two component signal transduction system that has been shown to affect virulence factor gene transcription (FIG. 1, item 8) (Fournier et al., 2001). Mutations in arlRS increase the transcription of hla, hlb, ssp, and spa. The observed up-regulation of gene transcription in the mutant strain is reflected in the secreted products. Analysis of mutant strains showed that an ArlSR mutation increases synthesis of agr RNAII and RNAIII and decreases the synthesis of SarA.

Environmental Conditions and virulence factor regulation. S. aureus can interpret a variety of environmental signals that modulate virulence factor production. Oxygen and carbon dioxide levels (Yarwood and Schlievert. 2000), osmolarity, glucose levels and pH (Regassa and Bentley, 1992; Regassa et al., 1992), magnesium concentration (Mills et al., 1996), heat (Bergdall, 1989), ethanol (Yu and Petrov, 1990), detergents (Fujimoto and Bales, 1998), antibiotics (Kernodle et al., 1995), as well as other conditions and compounds alter toxin production (Bergdall, 1989; Chan and Foster, 1998; Kernodle et al., 1995). With the exception of the induction of σ^{B}, and perhaps through the function of a respiratory locus with homology to Bacillus subtilis resD, the triggering of virulence factor production by environmental signals is not understood (Kullik et al., 1998; Thomson et al., 1997; Yarwood and Schlievert, 2000).

RNAIII and virulence factor regulation. RNAIII is required for decreased transcription of cell-surface protein genes and increased transcription of extracellular protein genes (Janzon and Arvidson, 1990; Morfeldt et al., 1996; Novick et al., 1993). While the effect of RNAIII is primarily on transcription, in the case of one extracellular protein, α-toxin, RNAIII is also required for translation (Morfeldt et al., 1995; Novick et al., 1993). Genetic evidence has involved RNAIII itself in the regulation of virulence factor genes. Synthesis of RNAIII from an agrBDCA-independent promoter in an agr-null mutant strain returns a wild-type pattern, although not levels, of virulence factor messages and translation products (Vandenesch et al., 1991). Mutational analysis of RNAIII has ruled out involvement of 6-toxin, the only known translation product of RNAIII.

How RNAIII functions to alter the transcription of virulence factor genes remains unknown. A comparison of migration patterns in denaturing and non-denaturing gels has demonstrated that RNAIII complexes with unidentified proteins (Morfeldt et al., 1995). RNAIII may be viewed as part of a ribonucleic acid-protein complex that is required for the transcription of staphylococcal virulence factor genes (Novick, 1995). Alternatively, RNAIII may act as an antagonist of a global repressors (McNamara et al., 2000). The RNA molecule DsrA-RNA is known to increase the transcription of genes that are suppressed in Escherichia coli by the histone-like silencer H-NS (Lease et al., 1998; Sledjeski et al., 1996). DrsA-RNA is part of a complex that binds H-NS and Hfq relieving DNA secondary structure that inhibits the transcription of the regulated genes. The emerging picture of riboreguation involves regulatory RNA coupled with protein components. In addition to DrsA-RNA, examples of this phenomenon are seen with oxys (Altuvia and Wagner, 2000), tmRNA (Karzai et al., 1999), CsrB (Romeo, 1998), and RNase P (Gopalan et al., 2001).

The mechanism by which RNAIII regulates the translation of the hla message has been the subject of one detailed study (Morfeldt et al., 1995). In the absence of RNAIII, secondary structure in the leader sequence at the 5′-end of the hla message blocks the ribosomal binding site, preventing the initiation of translation. When RNAIII is present, it hybridizes with the leader sequence of the message. This interaction causes a change in secondary structure within the leader that exposes the ribosomal binding allowing for the initiation of translation. Of note, a high level of DsrA-RNA expression is required for the translation of mRNA encoding σ^{S}, a stationary phase/stress E. coli sigma factor (Sledjeski et al., 1996). This observation is consistent with the fact that only high levels of RNAIII restore α-toxin activity to agr-minus strains (Janzon and Arvidson, 1990).

Clinical consequences. From a teleological perspective, the coordinate regulation of virulence factors by a pheromone-sensing signal transduction system is thought to abet staphylococcal pathogenesis. As described by Novick (1995), the early expression of cell-surface proteins can be imagined to augment the establishment of infection. In the absence of the pheromone, proteins that mediate adherence of the bacteria to host tissues and that aid the bacteria in circumventing host defenses are expressed. While encapsulated in micro-abscesses, the bacteria replicate. The environment surrounding the bacteria becomes more hostile as oxygen levels and pH values decrease, and glucose becomes limiting. In addition to these environmental signals for toxin production, the pheromone level surrounding the microbial cells reaches a critical concentration. At this point, the synthesis of surface proteins is down-regulated and the production of soluble enzymes (e.g. proteases) and toxins are up-regulated. The soluble proteins can be envisioned to be required for breaching the host defenses and allowing for bacterial dissemination. While the temporal aspect of the regulation of virulence factors have not yet been (and may never be) experimentally validated (Goerke et al., 2000), the general importance of the agr/sar-encoded pathway to the pathogenesis of S. aureus has been demonstrated in several animal models of staphylococcal disease (Abdelnour et al., 1993; Cheung et al., 1994; Darouiche et al., 1997; Gillaspy et al., 1995).

### SUMMARY OF THE INVENTION

The present invention describes genetic loci associated with the regulation of virulent factor expression in S. aureus. Important aspects of the present invention include nucleic acids, proteins, recombinant organisms, antibodies, kits, methods of detecting a virulent organism and of determining a compound capable of affecting virulence factor expression. Furthermore, the compositions of the present invention may be used in therapeutic and prophylactic methods to treating and preventing S. aureus-related diseases and disorders.

This invention provides S. aureus rot gene polynucleotide sequences, Rot polypeptides encoded by these sequences, antibodies that bind to these polypeptides, compositions comprising any of the above, as well as methods using the polynucleotides, polypeptides, and/or antibodies.

This invention provides S. aureus rlp gene polynucleotide sequences, Rlp polypeptides encoded by these sequences, antibodies that bind to these polypeptides, compositions comprising any of the above, as well as methods using the polynucleotides, polypeptides, and/or antibodies.

Accordingly, in one aspect, the invention includes an isolated polynucleotide comprising a member selected from the group consisting of:

(a) a polynucleotide of SEQ ID NO:1, SEQ ID NO:3, or SEQ ID NO:5 or a complement of SEQ ID NO:1, SEQ ID NO:3, or SEQ ID NO:5;

(b) a fragment of the polynucleotide of SEQ ID NO:1, SEQ ID NO:3, or SEQ ID NO:5 wherein the fragment comprises at least 20 contiguous nucleotides of SEQ ID NO:1, SEQ ID NO:3, or SEQ ID NO:5;

(c) a polynucleotide having at least 70% sequence identity to the sequence of SEQ ID NO:1, SEQ ID NO:3, or SEQ ID NO:5;

(d) a polynucleotide that hybridizes to any one of the polynucleotides of (a), (b) or (c) under conditions of 5× SSC, 50% formamide and 42° C., and which encodes a protein having the same biological function; and

(e) a polynucleotide encoding the same amino acid sequence as any of the polynucleotides of (a), (b), (c) or (d) but which exhibits regular degeneracy in accordance with the degeneracy of the genetic code.

In another aspect, the invention includes a recombinant polynucleotide comprising a member selected from the group consisting of:

(a) a polynucleotide of SEQ ID NO:1, SEQ ID NO:3, or SEQ ID NO:5 or a complement of SEQ ID NO:1, SEQ ID NO:3, or SEQ ID NO:5;

(b) a fragment of the polynucleotide of SEQ ID NO:1, SEQ ID NO:3, or SEQ ID NO:5 wherein the fragment comprises at least 20 contiguous nucleotides of SEQ ID NO:1, SEQ ID NO:3, or SEQ ID NO:5;

(c) a polynucleotide having at least 70% sequence identity to the sequence of SEQ ID NO:1, SEQ ID NO:3, or SEQ ID NO:5;

(d) a polynucleotide that hybridizes to any of the polynucleotides of (a), (b) or (c) under conditions of 5× SSC, 50% formamide and 42° C., and which encodes a protein having the same biological function; and

(e) a polynucleotide encoding the same amino acid sequence as any of the polynucleotides of (a), (b), (c) or (d) but which exhibits regular degeneracy in accordance with the degeneracy of the genetic code.

In a further aspect, the invention includes a protein or polypeptide encoded by the polynucleotide selected from the group consisting of

(a) a polynucleotide of SEQ ID NO:3 or a complement of SEQ ID NO:3;

(b) a fragment of the polynucleotide of SEQ ID NO:3 wherein the fragment comprises at least 20 contiguous nucleotides of SEQ ID NO:3;

(c) a polynucleotide having at least 70% sequence identity to the sequence of SEQ ID NO:3;

(d) a polynucleotide that hybridizes to any of the polynucleotides of (a), (b) or (c) under conditions of 5× SSC, 50% formamide and 42° C., and which encodes a protein having the same biological function; and

(e) a polynucleotide encoding the same amino acid sequence as any of the polynucleotides of (a), (b), (c) or (d) but which exhibits regular degeneracy in accordance with the degeneracy of the genetic code.

In another aspect, the invention includes an isolated protein or polypeptide comprising the amino acid of SEQ ID NO:2.

In a further aspect, the invention includes a recombinant protein or polypeptide comprising the amino acid of SEQ ID NO:2.

In a further aspect, the invention includes an isolated protein or polypeptide comprising a Rot polypeptide sequence from Staphylococcus aureus, wherein the polypeptide comprises at least 15 contiguous amino acids of SEQ ID NO:2.

In a further aspect, the invention includes an isolated protein or polypeptide comprising the amino acid of SEQ ID NO:4.

In a further aspect, the invention includes a recombinant protein or polypeptide comprising the amino acid of SEQ ID NO:4.

In a further aspect, the invention includes an isolated protein or polypeptide comprising a Rlp polypeptide sequence from Staphylococcus aureus, wherein the polypeptide comprises at least 15 contiguous amino acids of SEQ ID NO:4.

In a further aspect, the invention includes cloning vectors, expression vectors, host cells, fusion proteins, nucleic acid primers and compositions comprising any of the above mentioned polynucleotides.

In a further aspect, the invention includes compositions comprising any of the above mentioned polypeptides.

In a further aspect, the invention includes purified antibodies that are capable of specifically binding to a polypeptide of the invention.

In a further aspect, the invention includes a monoclonal antibody capable of specifically binding to a polypeptide of the invention.

In a further aspect, the invention includes kits for detection or quantification of any of the polynucleotides or polypeptides of the invention.

### BRIEF DESCRIPTION OF THE FIGURES

FIG. 1. A Model for the regulation of staphylococcal virulence factors. A single S. aureus is delineated by the large hatched circle. Genes are represented by boxes and promoters are labeled “P”. With the exception of RNAIII (arrow and ladder-like structure) and the α-toxin message (arrow), mRNA is depicted using grey straight lines with triangular arrowheads. A detailed summary of FIG. 1 can be found in the Detailed Description of the Invention.

FIG. 2. (Panel A) An EcoRI(E) restriction map of the wild type allele of agr in RN6390 (top) and the agr-null allele (Δagr) in PM466 (bottom). The agr P2 operon and P3 transcript are represented by the striped and shade boxes, respectively. Chromosomal DNA on the 3′-end of the P2 operon and 3′-end of the P3 transcript are represented by a black and grey lines, respectively. (Panel B) Chromosomal DNA from RN6390 (lane 1) and PM466 (lane 2) digested with EcoRI and analyzed by Southern hybridization using agr and flanking ssDNA as the probe. DNA hybridizing to agr-flanking regions is marked with arrows. Abbreviations: C: ClaI; H: HincII; H/C: ligation of a HincII site with a T₄ polymerase blunt-ended ClaI site.

FIG. 3. BLASTP alignment of the rot gene product (Rot) and the S. aureus regulatory protein SarA, (e value=5.9) and the S. epidermidis SarA (e value=0.49). Rot was used as the query sequence against the non-redundant database at the National Center for Biotechnology Information. Identities are shown, (+) denotes similarity, and numbers at right refer to amino acids in the respective proteins.

FIG. 4. Quantitative measurements of α-toxin activity in supernatant fluids from post-exponential phase (10 hour) cultures of S. aureus strains RN6390 (wild type, gray bar), PM466 (vertically striped bar), PM614 (white bar), PM720 (diagonal striped bar), and PM702 (black bar). Relevant genotypes are shown (wt: wild type; Δagr: agr-null allele, rot: wild type rot allele; rot::Tn917: insertionally inactivated rot).

FIG. 5. Northern analysis of the α-toxin message in RNA from post-exponential phase cultures of S. aureus strains (Panel A) PM466 (Panel B) PM614, and (Panel C) PM720. Lane 1 contains 30 μg of total RNA serially diluted (1:2) in lanes 2-7. The transcript was identified by hybridization using digoxigenin-labeled probe specific for the gene encoding α-toxin with chemiluminescent chemistry.

FIG. 6. BLASTP alignment of the gene products of rlp and sarA. Identities are shown, (+) denotes similarity, and numbers at right refer to amino acids in the respective proteins.

FIG. 7. Southern analysis of chromosomal DNA from S. aureus strains PM734, PM743, and RN6390. A 10 kb PstI fragment hybridized with labeled rlp in the wild-type strain RN6390 (lane 1). This is in contrast to the 12 kb fragment seen in the mutant strain, PM734, in which rlp is interrupted by a 2 kb erythromycin-encoding cassette (rlp::erm) that does not encode a PstI site (lane 3). Lane 2 contains PstI-digested chromosomal DNA from strain PM743, PM734 with a the rlp::erm allele restored to wild-type rlp by allelic exchange. rlp is now also known as sarU by agreement among the Staphylococcus aureus researchers.

FIG. 8. Activity data from select extracellular and cell surface staphylococcal virulence-associated proteins. (Panel A) cell-surface coagulase, measured as reciprocal of the dilution of cultures standardized by optical density that formed a clot in rabbit plasma (Smeltzer et al., 1993); (Panel B) α-toxin, measured as the reciprocal of the dilution of culture supernatant fluids from a standard number of bacteria that yielded 50% lysis, (Panel C) total proteolytic activity measured azocasien hydrolysis (Smeltzer et al., 1993), and (Panel D) extracellular protein A measured by ELISA. Data from an agr-minus strain is included for reference. Activity and protein levels were measured in S. aureus strains RN6390 (wild-typ), PM734 (sarU::ermC), PM743 (sarU-restored), and PM466 (Dagr).

FIG. 9. Primer extension analysis of the α-toxin (hla) and protein A (spa) messages (panel A), RNAII and RNAIII (panel B), and sar (panel C), in RNA from exponential phase cultures (lanes 1-3 and 4-6, respectively) of S. aureus strains RN6390 (lanes 1 and 4), PM734 (lanes 2 and 5) and PM743 (lanes 3 and 6). Relative levels of specific messages calculated as the area in square pixels are given above each band with the exception of the smaller spa primer extension products which are listed below.

FIG. 10. Nucleic acid sequence (SEQ ID NO:12) containing the rot gene. The −35 and TATA box sequences are underlined and indicated by (−35) and (−10), respectively. Ts indicates the putative transcriptional start site. The Rot open-reading frame is indicated by segmenting the sequence into codons and the encoded amino acids are shown below their respective codons. The translational stop codon is indicated by *. Putative transcriptional termination sequences are underline in the 3′ region of the sequence.

FIG. 11. A Clustal alignment of the amino acids, represented by the single letter code, of the S. aureus strain N315 Sar-homologues. The sequences of the characterized Sar-homologues are labeled. Uncharacterized Sar-homologues are labeled with the GenBank numbers assigned to proteins from strain N315. The degree of similarity of the amino acids in the proteins, from low to high, is displayed as periods, colons, and asterisks. Note that SarU, SarS, and SA2091 have two regions of homology with the other Sar-homologues. This region encompassed the “DER” sequence and has homology with the helix-turn-helix wing region of the putative DNA-binding domain of MarR (McNamara et al., 2000).

### DETAILED DESCRIPTION OF THE INVENTION

Staphylococcus aureus remains an important human pathogen responsible for a broad spectrum of infections, intoxications, and syndromes. Due to the emergence of multiple-antibiotic resistant strains, new agents are required to combat these conditions. One possible target for new antibiotics is regulatory molecules that govern virulence factor production. To date, the products of several genetically unlinked loci have been found to coordinately regulate virulence factor expression.

Described herein are nucleic acids and polypeptides that affect the expression of virulence factors in S. aureus. Rot (SEQ ID NO:2) is encoded by the nucleic acid of SEQ ID NO:1. As described herein, Rot is a global repressor of toxins in S. aureus (See Example 1). Rlp (SEQ ID NO:4) is encoded by the nucleic acid of SEQ ID NO:3. As described herein, Rlp is a virulence factor gene regulator in S. aureus (See Example 2); rlp is now also known as sarU by agreement among the Staphylococcus aureus researchers.

Methods of detecting Rot or Rlp or Rot-encoding or Rlp-encoding nucleic acids may be useful in the diagnosis or prognosis of S. aureus infection. Furthermore, such methods may also be useful in screening for compounds or conditions that affect virulence factor expression in S. aureus. Compounds found to affect virulence factor expression by the methods of the present invention may have particular utility as antibiotics to S. aureus or related organisms. For example, a compound found to activate rot expression may be useful in methods of inhibiting the production of toxic shock syndrome toxin-1 (TSST-1). Similarly, a compound that blocks the activity of Rlp would stop toxin production in S. aureus by preventing the activation of the agr/sar pathway. Such methods would be effective at ameliorating the progression of toxic shock syndrome in a patient. Other S. aureus-related infections, diseases, and syndromes that may be treated by the compounds and methods of the present invention include, but are not limited to, skin and wound infections, tissue abscesses, folliculitis, food poisoning, osteomyelitis, pneumonia, scalded skin syndrome, septicaemia, septic arthritis, myocarditis, and endocarditis.

Nucleic Acids

Important aspects of the present invention concern isolated nucleic acid segments and recombinant vectors encoding Rot and Rlp. Because these proteins are shown herein to affect virulence factor expression, nucleic acid segments and recombinant vectors encoding them are particularly useful. For example, they are useful in methods of determining compounds or conditions that affect the expression of virulence factors.

In one aspect, the invention concerns the creation and use of recombinant host cells through the application of DNA technology that express Rot or Rlp. In specific embodiments, these technologies using rot or rlp may comprise the sequence of SEQ ID NO:1 or SEQ ID NO:3, respectively. In some embodiments, the nucleic acid is a DNA segment. The DNA segment may be genomic or a cDNA segment. Further, it is contemplated that RNA and protein nucleic acids encoding rot or rlp also are within the scope of the present invention.

In one embodiment, the present invention concerns DNA segments that are free from total genomic DNA. It is contemplated that such DNA segments are capable of expressing a protein or polypeptide that affects virulence factor expression in the cell expressing the DNA segment.

As used herein, the term “DNA segment” refers to a DNA molecule that has been isolated free of total genomic DNA of a particular species. Therefore, a DNA segment encoding rot or rlp refers to a DNA segment that contains Rot or Rlp coding sequences yet is isolated away from, or purified free from, total genomic DNA. Included within the term “DNA segment”, are DNA segments and smaller fragments of such segments, and also recombinant vectors, including, for example, plasmids, cosmids, phage, viruses, and the like.

Similarly, a DNA segment comprising an isolated or purified rot or rlp refers to a DNA segment including Rot or Rlp coding sequences and, in certain aspects, regulatory sequences, isolated substantially away from other naturally occurring genes or protein encoding sequences. As will be understood by those in the art, this includes both genomic sequences, cDNA sequences and smaller engineered gene segments that express, or may be adapted to express, proteins, polypeptides, domains, peptides, fusion proteins and mutants.

“Isolated substantially away from other coding sequences” means that the segment of interest forms the significant part of the coding region of the DNA segment, and that the DNA segment does not contain large portions of naturally-occurring coding DNA, such as large chromosomal fragments or other functional genes or open reading frame coding regions. Of course, this refers to the DNA segment as originally isolated, and does not exclude genes or coding regions later added to the segment by the hand of man.

In particular embodiments, the invention concerns isolated DNA segments and recombinant vectors incorporating DNA sequences that encode Rot or Rlp protein or polypeptide that includes within its amino acid sequence a contiguous amino acid sequence in accordance with, or essentially as set forth in, SEQ ID NO:2 or SEQ ID NO:4, respectively. Moreover, in other particular embodiments, the invention concerns isolated DNA segments and recombinant vectors that encode a Rot or Rlp protein or polypeptide that includes within its amino acid sequence the substantially full length protein sequence of SEQ ID NO:2 or SEQ ID NO:4, respectively.

The term “biologically functional equivalent” is well understood in the art and is further defined in detail herein. Accordingly, sequences that have been between about 70% and about 80%, or more preferably, between 81% and about 90%; or even more preferably, between about 91% and about 99%; of amino acids that are identical or functionally equivalent to the amino acids of SEQ ID NO:2 will be sequences that are “essentially as set forth in SEQ ID NO:2”, provided the biological activity of the protein is maintained.

In certain other embodiments, the invention concerns isolated DNA segments and recombinant vectors that include within their sequence a nucleic acid sequence essentially as set forth in SEQ ID NO:1 or SEQ ID NO:3. The term “essentially as set forth in SEQ ID NO:” is used in the same sense as described above and means that the nucleic acid sequence substantially corresponds to a portion of SEQ ID NO:1 and has relatively few codons that are not identical, or functionally equivalent, to the codons of SEQ ID NO:1. DNA segments that encode proteins capable of affecting expression of virulence factors in cells expressing the DNA segment will be most preferred.

The term “functionally equivalent codon” is used herein to refer to codons that encode the same amino acid, such as the six codons for arginine or serine, and also refers to codons that encode biologically equivalent amino acids (see Table 1).

TABLE 1

| | |
|---|---|
| Amino Acids | Codons |

| | | |
|---|---|---|
| Alanine | Ala A | GCT GCT GCA GCG |
| Cysteine | Cys C | TGC TGT |
| Aspartic acid | Asp D | GAC GAT |
| Glutamic acid | Glu E | GAG GAA |
| Phenylalanine | Phe F | TTC TTT |
| Glycine | Gly G | GGC GGG GGA GGT |
| Histidine | His H | CAC CAT |
| Isoleucine | Ile I | ATC ATT ATA |
| Lysine | Lys K | AkG AAA |
| Leucine | Leu L | CTG CTC TTG CTT CTA |
| | | TTA |
| Methionine | Met M | ATG |
| Asparagine | Asn N | AAC AAT |
| Proline | Pro P | CCC CCT CCA CCG |
| Glutarnine | Gln Q | CAG CAA |
| Arginine | Arg R | CGC AGG CGG AGA CGA |
| | | CGT |
| Serine | Ser S | AGC TCC TCT AGT TCA |
| | | TCG |
| Threonine | Thr T | ACC ACA ACT ACG |
| Valine | Val V | GTG GTC GTT GTA |
| Tryptophan | Trp W | TGG |
| Tyrosine | Tyr Y | TAC TAT |

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, e.g., tag sequences.

Excepting internal or flanking regions, and allowing for the degeneracy of the genetic code, sequences that have between about 70% and about 79%; or more preferably, between about 80% and about 89%; or even more preferably, between about 90%, and 92%, and about 99%; of nucleotides that are identical to the nucleotides of SEQ ID NO:1 will be sequences that are “essentially set forth in SEQ ID NO:1”.

Sequences that are essentially the same as those set forth in SEQ ID NO:1 may also be functionally defined as sequences that are capable of hybridizing to a nucleic acid segment containing the complement of SEQ ID NO:1 under relatively stringent conditions. Suitable relatively stringent hybridization conditions will be well known to those of skill in the art, as disclosed herein.

Naturally, the present invention also encompasses DNA segments that are complementary, or essentially complementary, to the sequence set forth in SEQ ID NO:1 or SEQ ID NO:3. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO:1 or SEQ ID NO:3 under relatively stringent conditions such as those described herein.

The nucleic acid segments of the present invention, regardless of the length of the coding sequence itself, may be combined with other DNA sequences, such as promoters, polyadenylation signals, additional restriction enzyme sites, multiple cloning segments, and the like, such that their overall length may vary considerably. It is therefore contemplated that a nucleic acid fragment of almost any length may be employed, with the total length preferably being limited by the ease of preparation and use in the intended recombinant DNA protocol.

For example, nucleic acid fragments may be prepared that include a short contiguous stretch identical to or complementary to SEQ ID NO:1 or SEQ ID NO:3, such as about 8, about 10 to 14, or about 15 to about 20 nucleotides, and that are up to about 20,000, or about 10,000, or about 5,000 base pairs in length with segments of about 3,000 being preferred in certain cases. DNA segments with total lengths of about 1,000, about 5,000, about 200, about 100 and about 50 base pairs in length (including all intermediate lengths) are also contemplated to be useful.

It will be readily understood that “intermediate lengths”, in these contexts, means any length between the quoted ranges, such as 10, 11, 12, 13, 14, 15, 16, 17, 18, 19, 20, 21, 22, 23, etc.; 30, 31, 32, 33, etc; 50, 51, 52, 53, etc.; 100, 101, 102, 103, etc.; 150, 151, 152, 153, etc.; including all integers through the 200-500; 500-1,000; 1,000-2,000; 2,000-3,000; 3,000-5,000; 5,000-10,000 ranges.

The various probes and primers designed around the disclosed nucleotide sequences of the present invention may be of any length. By assigning numeric values to a sequence, for example, the first residue is 1, the second residue is 2, etc., an algorithm defining all primers can be proposed;

n to n+y

wherein n is an integer from 1 to the last number of the sequence and y is the length of the primer minus one, where n+y does not exceed the last number of the sequence. Thus, for a 10-mer, the probes correspond to bases 1 to 10, 2 to 11, 3 to 12 . . . and so on. For a 15-mer, the probes correspond to bases 1 to 15, 2 to 16, 3 to 17 . . . and so on. For a 20-mer, the probes correspond to bases 1 to 20, 2 to 21, 3 to 22 . . . and so on.

It will also be understood that this invention is not limited to the particular nucleic acid sequence of SEQ ID NO:1 or SEQ ID NO:3. Recombinant vectors and isolated DNA segments may therefore variously include the coding regions themselves, coding regions bearing selected alterations or modifications in the basic coding region, or they may encode larger polypeptides that nevertheless include such coding regions or may encode biologically functional equivalent proteins or polypeptides that have variant amino acids sequences.

The DNA segments of the present invention encompass biologically functional equivalent proteins and polypeptides. Such sequences may arise as a consequence of codon redundancy and functional equivalency that are known to occur naturally with nucleic acid sequences and the proteins thus encoded. Alternatively, functionally equivalent proteins or polypeptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the antigenicity of the protein or to test mutants in order to examine transformation activity at the molecular level. Methods of site-directed mutagenesis are discussed herein.

One may also prepare fusion proteins and polypeptides, e.g. where the Rot or Rlp coding region is aligned within the same expression unit with other proteins or polypeptides having desired functions, such as for purification or immunodetection purposes (e.g., proteins that may be purified by affinity chromatography and enzyme label coding regions, respectively).

Encompassed by the invention are DNA segments encoding relatively small polypeptides, such as, for example, polypeptides of from about 15 to about 50 amino acids in lengths, and more preferably, of from about 20 to about 30 amino acids in length; and also larger polypeptides up to and including proteins corresponding to those encoded by the full-length sequence set forth in SEQ ID NO:1 or SEQ ID NO:3.

Recombinant Vectors, Host Cells and Expression

Recombinant vectors form further aspects of the present invention. The term “expression vector or construct” means any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a gene and translation of an RNA into a gene product. In other embodiments, expression only includes transcription of the nucleic acid, for example, to generate antisense constructs.

Particularly useful vectors are contemplated to be those vectors in which the coding portion of the DNA segment, whether encoding a full length protein or smaller polypeptide, is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned”, “under control”, “operably linked” or “under transcriptional control” means that the promoter is in the correct location and orientation in relation to the nucleic acid to control RNA polymerase initiation and expression of the gene.

The promoter may be in the form of the promoter that is naturally associated with rot or rlp, as may be obtained by isolating the 5′ non-coding sequences located upstream of the open reading frame, for example, using recombinant cloning and/or PCR technology, in connection with the compositions disclosed herein.

In preferred embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with rot or rlp in its natural environment. Such promoters may include promoters normally associated with other genes, and/or promoters isolated from any other bacterial, viral or eukaryotic.

Naturally, it will be important to employ a promoter that effectively directs the expression of the DNA segment in the cell type, or organism, chosen for expression. The use of promoter and cell type combinations for protein expression is generally known to those of skill in the art of molecular biology, for example, see Sambrook et al. (1989), incorporated herein by reference. The promoters employed may be constitutive, or inducible, and can be used under the appropriate conditions to direct high level expression of the introduced DNA segment, such as is advantageous in the large-scale production of recombinant proteins or polypeptides.

At least one module in a promoter functions to position the start site for RNA synthesis. The best known example of this is the TATA box, but in some promoters lacking a TATA box, such as the promoter for the mammalian terminal deoxynecleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

The particular promoter that is employed to control the expression of a nucleic acid is not believed to be critical, so long as it is capable of expressing the nucleic acid in the targeted cell or organism. Thus, where Staphylococcus is targeted, it may be preferable to position the nucleic acid coding region adjacent to and under the control of a promoter that is capable of being expressed in Staphylococcus. Generally speaking, such a promoter might include a staphylococcal or heterologous phage promoter.

In various other embodiments wherein the targeted cell is a mammalian cell, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters which are well-known in the art to achieve expression of a transgene is contemplated as well, provided that the levels of expression are sufficient for a given purpose.

As indicated, it is contemplated that one may use any regulatory element to express Rot or Rlp disclosed by the present invention; however, under certain circumstances it may be desirable to use the innate promoter region associated with the gene of interest to control its expression, such as the rot or rlp promoter within the 5′ flanking region of a genomic clone. As noted above, in most cases, genes are regulated at the level of transcription by regulatory elements that are located upstream, or 5′ to the genes.

In general, to identify regulatory elements for the gene of interest, one would obtain a genomic DNA segment corresponding to the region located between about 10 to 50 nucleotides up to about 2000 nucleotides or more upstream from the transcriptional start site of the gene, i.e., the nucleotides between positions −10 and −2000. A convenient method used to obtain such a sequence is to utilize restriction enzyme(s) to excise an appropriate DNA fragment. Restriction enzyme technology is commonly used in the art and will be generally known to the skilled artisan. For example, one may use a combination of enzymes from the extensive range of known restriction enzymes to digest the genomic DNA. Analysis of the digest fragments would determine which enzyme(s) produce the desired DNA fragment. If desired, one may even create a particular restriction site by genetic engineering for subsequence use in litigation strategies.

Alternatively, one may choose to prepare a series of DNA fragments differentiated by size through the use of a deletion assay with linearized DNA. In such an assay, enzymes are also to digest the genomic DNA; however, in this case, the enzymes do not recognize specific sites within the DNA but instead digest the DNA from the free end(s). In this case, a series of size differentiated DNA fragments can be achieved by stopping the enzyme reaction after specified time intervals. Of course, one may also choose to use a combination of both restriction enzyme digestion and deletion assay to obtain the desired DNA fragment(s). Furthermore, one of skill in the art would understand that there are many techniques that may be used to obtain a DNA fragment (e.g., PCR) and the invention is not limited by the technique used to obtain the fragment.

Once the desired DNA fragment has been isolated, its potential to regulate a gene and determine the basic regulatory unit may be examined using any one of several conventional techniques. It is recognized that once the core regulatory region is identified, one may choose to employ a longer sequence that comprises the identified regulatory unit. This is because although the core region is all that is ultimately required, it is believed that particular advantages accrue, in terms of regulation and level of induction achieved where one employs sequences which correspond to the natural control regions over long regions, e.g., from around 25 or so nucleotides to as many as 1000 to 1500 or so nucleotides in length. The preferred length will be in part determined by the type of expression system used and the results desired.

Numerous methods are known in the art for precisely locating regulatory units with larger DNA sequences. Most conveniently, the desired control sequence is isolated within a DNA fragment(s) that is subsequently modified using DNA synthesis techniques to add restriction site linkers to the fragment(s) termini. This modification readily allows the insertion of the modified DNA fragment into an expression cassette that contains a reporter gene that confers on its recombinant host cell a readily detectable phenotype that is either expressed or inhibited, as may be the case.

Generally, reporter genes encode a polypeptide not otherwise produced by the host cell; or a protein or factor produced by the host cell but at much lower levels; or a mutant form of a polypeptide not otherwise produced by the host cell. Preferably, the reporter gene codes an enzyme which produces a calorimetric or fluorometic change in the host cell which is detectable by in situ analysis and is a quantitative or semi-quantitative function of transcriptional activation. Exemplary reporter genes encode esterases, phosphatases, proteases and other proteins detected by activity that generate a chromophore or fluorophore as will be known to the skilled artisan. Well-known examples of such a reporter gene are E. coli β-galactosidase, luciferase and chloramphenicol-acetyl-transferase (CAT). Alternatively, a reporter gene may render its host cell resistant to a selection agent. For example, the gene neo renders cells resistant to the antibiotic neomycin. It is contemplated that virtually any host cell system compatible with the reporter gene cassette may be used to determine the regulatory unit. Thus mammalian or other eukaryotic cells, insect, bacterial or plant cells may be used.

Once a DNA fragment containing the putative regulatory region is inserted into an expression cassette that is in turn inserted into an appropriate host cell system, using any of the techniques commonly known to those of skill in the art, the ability of the fragment to regulate the expression of the reporter gene is assessed. By using a quantitative reporter assay and analyzing a series of DNA fragments of decreasing size, for example produced by convenient restriction endonuclease sites, or through the actions of enzymes such as BAL31, E. coli exonuclease III or mung bean nuclease, and which overlap each other a specific number of nucleotides, one may determine both the size and location of the native regulatory unit.

Of course, once the core regulatory unit has been determined, one may choose to modify the regulatory unit by mutating certain nucleotides within the core unit. The effects of these modifications may be analyzed using the same reporter assay to determine whether the modifications either enhance or reduce transcription. Thus, key nucleotides within the core regulatory sequence can be identified.

It is recognized that regulatory units often contain both elements that either enhance or inhibit transcription. In the case that a regulatory unit is suspected of containing both types of elements, one may use competitive DNA mobility shift assays to separately identify each element. Those of skill in the art will be familiar with the use of DNA mobility shift assays.

It may also be desirable to modify the identified regulatory unit by adding additional sequences to the unit. The added sequences may include additional enhancers, promoters or even other genes. Thus, one may, for example, prepare a DNA fragment that contains the native regulatory elements positioned to regulate one or more copies of the negative gene and/or another gene or prepare a DNA fragment which contains not one but multiple copies of the promoter region such that transcription levels of the desired gene are relatively increased.

A specific initiation signal also may be required for efficient translation of coding sequences. These signals include an ATG initiation code and adjacent sequences. Exogenous translational control signals, including an ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. Furthermore, it is well known that S. aureus are capable of utilizing alternative (non-ATG) start sites and, thus, initiation signals including such alternative sites are also contemplated. The exogenous translational control signals and initiation codons can be either natural or synthetic. The efficiency of expression may be enhanced by the inclusion of appropriate transcription enhancer elements.

It is proposed that rot or rlp may be co-expressed with another gene, wherein the proteins may be co-expressed in the same cell. Co-expression may be achieved by co-transfecting the cell with two distinct recombinant vectors, each bearing a copy of either the respective DNA. Alternatively, a single recombinant vector may be constructed to include the coding regions for both of the proteins, which could then be expressed in cells transfected or transformed with the single vector. In either event, the term “co-expression” herein refers to the expression of both the polypeptide comprising the amino acid sequence SEQ ID NO:2 or SEQ ID NO:4 and other protein in the same recombinant cell. In one embodiment, the polypeptides comprising the amino acid sequence SEQ ID NO:2 and SEQID NO:4 are expressed in the same recombinant cell.

As used herein, the terms “engineered” and “recombinant” cells are intended to refer to a cell into which an exogenous DNA segment or gene, such a gene coding Rot or Rlp has been introduced. Therefore, engineered cells are distinguishable from naturally occurring cells that do not contain a recombinantly introduced exogenous DNA segment or gene. Engineered cells are thus cells having a gene or genes introduced through the hand of man. Recombinant cells include those having an introduced cDNA or genomic gene, and also include genes positioned adjacent to a promoter not naturally associated with the particular introduced gene.

To express a recombinant Rot or Rlp in accordance with the present invention, one would prepare an expression vector that comprises an Rot- or Rlp-encoding nucleic acid under the control of one or more promoters. To bring a coding sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded recombinant protein. This is the meaning of “recombinant expression” in this context.

Many standard techniques are available to construct expression vectors containing the appropriate nucleic acids and transcriptional/translational control sequences in order to achieve protein or polypeptide expression in a variety of host-expression systems. Cell types available for expression include, but are not limited to, bacteria, such as S. aureus, E. coli and B. subtilis transformed with recombinant bacteriophage DNA, plasmid DNA or cosmid DNA expression vectors.

In general, plasmid vectors containing replicon and control sequences that are derived from species compatible with the host cell are used in connection with these hosts. The vector ordinarily carries a replication site, as well as marking sequences that are capable of providing phenotypic selection in transformed cells. For example, E. coli is often transformed using derivatives of pBR322, a plasmid derived from E. coli species. PBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector that can be used to transform host cells, such as E. coli LE392.

Further useful vectors include pIN vectors (Inouye and Inouye, 1985); pQE (His-tagged) vectors (Qiagen) and pGEX vectors, for use in generating glutathione-S-transferase (GST) soluble fusion proteins for later purification, separation or cleavage. Other suitable fusion proteins are those with β-galactosidase, ubiquitin and the like.

It is contemplated that a polypeptide of the invention may be “overexpressed”, i.e., expressed in increasing levels of relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein or polypeptide in comparison to the level in natural cells is indicative of overexpression, as is a relative abundance of the specific protein in relation to the other proteins produced by the host cell and, e.g., visible on a gel.

Nucleic Acid Detection

In addition to their use in directing the expression Rot or Rlp encoded polypeptides, the nucleic acid sequences disclosed herein also have a variety of other uses. For example, they also have utility as probes or primers in nucleic acid hybridization embodiments. They may be particularly useful in methods and kits for the detection of Staphylococcus aureus.

Hybridization

The use of a hybridization probe of between 17 and 300 nucleotides in length allows the formation of a duplex molecule that is both stable and selective. Molecules have complementary sequences over stretches greater than 20 bases in length are generally preferred, in order to increase stability and selectivity of the hybrid, and thereby improve the quality and degree of particular hybrid molecules obtained. One will generally prefer to design nucleic acid molecules having stretches of 20 to 30 nucleotides, or even longer where desired. Such fragments may be readily prepared by, for example, directly synthesizing the fragment by chemical means or by introducing selected sequences into recombinant vectors for recombinant production.

Accordingly, the nucleotide sequences of the invention may be used for their ability to selectively form duplex molecules with complementary stretches of genes or RNAs or to provide primers for amplification of DNA or RNA from an organism. Depending on the application envisioned, one would desire to employ varying conditions of hybridization to achieve varying degrees of selectivity of probe towards target sequence.

For applications requiring high selectivity, one would typically desire to employ relatively stringent conditions to form the hybrids, e.g., one will select relatively low salt and/or high temperature conditions, such as provided by about 0.02 M to about 0.10 M NaCl at temperatures of about 50 [symbol] C. to about 70 [symbol] C. Such high stringency conditions tolerate little, if any, mismatch between the probe and the template or target strand, and would be particularly suitable for isolating specific genes or detecting specific mRNA transcripts. It is appreciated that conditions can be rendered more stringent by the addition of increasing amounts of formamide.

For certain applications, for example, substitution of nucleotides by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37 [symbol] C. to about 55 [symbol] C., while a low stringency condition could be provided by about 0.15 M to about 0.9 M salt, at temperatures ranging from about 20 [symbol] C. to about 55 [symbol] C. Thus, hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20 [symbol] C. to about 37 [symbol] C. Other hybridization conditions utilized could include approximately 10 mM Tris-HCl (pH 8.3), 50 mM KCl, 1.5 mM MgCl₂, at temperatures ranging from approximately 40 [symbol] C. to about 72 [symbol] C.

In certain embodiments, it will be advantageous to employ nucleic acid sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known that can be employed to provide a detection means visible to the human eye or spectrophotometrically, to identify specific hybridization with complementary nucleic acid-containing samples.

In general, it is envisioned that the hybridization probes described herein will be useful both as reagents in solution hybridization, as in PCR, for detection of expression of corresponding genes, as well as in embodiments employing a solid phase. In embodiments involving a solid phase, the test DNA (or RNA) is absorbed or otherwise affixed to a selected matrix or surface. This fixed, single-stranded nucleic acid is then subjected to hybridization with selected probes under desired conditions. The selected conditions will depend on the particular circumstances based on the particular criteria required (depending, for example, on the G+C content, type of target nucleic acid, source of nucleic acid, size of hybridization probe, etc). Following washing of the hybridized surface to remove non-specifically bound probe molecules, hybridization is detected, or even quantified, by means of the label.

Amplification and PCR

Nucleic acid used as a template for amplification is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA and is used directly as the template for amplification.

Pairs of primers that selectively hybridize to nucleic acids corresponding to rot or rlp are contacted with the isolated nucleic acid under conditions that permit selective hybridization. The term “primer”, as defined herein, is meant to encompass any nucleic acid that is capable of priming the synthesis of a nascent nucleic acid in a template-dependent process. Typically, primers are oligonucleotides from ten to thirty base pairs in length, but longer sequences can be employed. Primers may be provided in double-stranded or single-stranded form, although the single-stranded form is preferred.

Once hybridized, the nucleic acid primer complex is contacted with one or more enzymes that facilitate template-dependent nucleic acid synthesis. Multiple rounds of amplification, also referred to as “cycles”, are conducted until a sufficient amount of amplification product is produced.

Next, the amplification product is detected. In certain applications, the detection may be performed by visual means. Alternatively, the detection may involve indirect identification of the product via chemiluminescence, radioactive scintography of incorporated radiolabel or fluorescent label or even via a system using electrical or thermal impulse signals (Affymax technology).

A number of template dependent processes are available to amplify the marker sequences present in a given template sample. One of the best known amplification methods is the polymerase chain reaction (referred to as PCR) which is described in detail in U.S. Pat. Nos. 4,683,195, 4,683,202 and 4,800,159, and each incorporated herein by reference in entirety.

Following any amplification, it may be desirable to separate the amplification product from the template and the excess primer for the purpose of determining whether specific amplification has occurred. In one embodiment, amplification products are separated by agarose, agarose-acrylamide, or polyacrylamide gel electrophoresis using standard methods (Sambrook et al., 1989).

Alternatively, chromatographic techniques may be employed to effect separation. There are many kinds of chromatography which may be used in the present invention: absorption, partition, ion-exchange and molecular sieve, and many specialized techniques for using them including column, paper, thin-layer and gas chromatography.

Amplification products must be visualized in order to conform amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled, nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

All the essential materials and reagents required for detecting rot or rlp in a biological sample may be assembled together in a kit. This generally will comprise preselected primers for specific markers. Also included may be enzymes suitable for amplifying nucleic acids including various polymerases (RT, Taq., etc), deoxynucleotides and buffers to provide the necessary reaction mixture for amplification.

Such kits generally will comprise, in suitable means, distinct containers for each individual reagent and enzyme as well as for each marker primer pair. In some embodiments, pairs of primers for amplifying nucleic acids are selected to amplify the sequences specified in SEQ ID NO:1 or SEQ ID NO:3.

In other embodiment, the kit comprises the components for the detection of Rot or Rlp encoding nucleic acids by an RNase protection assay. The RNase protection assay was first used to detect and map the ends of specific mRNA targets in solution. The assay relies on being able to easily generate high specific activity radiolabeled RNA probes complementary to the mRNA of interest by in vitro transcription were recombinant plasmids containing bacteriophage promoters. The probes are mixed with total cellular RNA samples to permit hybridization to their complementary targets, then the mixture is treated with RNase to degrade excess unhybridized probe. Also, as originally intended, the RNase used is specific for single-stranded RNA, so that hybridized double-stranded probe is protected from degradation. After inactivation and removal of the RNase, the protected probe (which is proportional in amount to the amount of target mRNA that was present) is recovered and analyzed on a polyacrylamide gel.

Proteins and Polypeptides

The present invention provided purified, and in preferred embodiments, substantially purified, proteins and polypeptides comprising the amino acid sequence of SEQ ID NO:2 or SEQ ID NO:4. The term “purified protein or polypeptide” as used herein is intended to refer to an aqueous composition, isolatable from S. aureus cells or recombinant host cells, wherein the protein or polypeptide is purified to any degree relative to its naturally-obtainable state, i.e., relative to its purity within a cellular extract. A purified protein or polypeptide therefore also refers to a protein or polypeptide free from the environment in which it naturally occurs.

Proteins and polypeptides comprising the amino acid sequence of SEQ ID NO:2 or SEQ ID NO:4 may be full length proteins, preferably Rot or Rlp, respectively. Proteins and polypeptides comprising the amino acid sequence of SEQ ID NO:2 or SEQ ID NO:4 may also be less than full length proteins, such as individual domains, regions or even epitopic peptides. Where less than full length proteins are concerned the most preferred will be those containing predicted immunogenic sites and/or those containing the functional domains. Such polypeptides may contain at least 5, 6, 7, 8, 9, 10, 11, 12, 13, 14, 15, 16, 17, 18, 19 20, 25, 30, 35, 40, 50, 75, 100 or more contiguous amino acids from SEQ ID NO:2 or SEQ ID NO:4. Of course, one of skill in the art would understand that the polypeptide lengths provided above are for example only and essentially any length contiguous amino acid sequence from SEQ ID NO:2 or SEQ ID NO:4 may be included as proteins or polypeptides of the present invention.

Generally, “purified” will refer to a protein or polypeptide composition that has been subjected to fractionation to remove various protein or polypeptide components not comprising the amino acid sequence of SEQ ID NO:2 or SEQ ID NO:4, and which composition substantially retains its activity, as may be assessed by binding to amino acid sequence of SEQ ID NO:2- or SEQ ID NO:4-specific antibodies.

Where the term “substantially purified” is used, this will refer to a composition in which the protein or polypeptide forms the major component of the composition, such as constituting about 50% of the proteins in the composition or more. In preferred embodiments, a substantially purified protein will constitute more than 60%, 70%, 80%, 90%, 95%, 99% or even more of the proteins in the composition.

A polypeptide or protein that is “purified to homogeneity,” as applied to the present invention, means that the polypeptide or protein has a level or purity where the polypeptide or protein is substantially free from other proteins and biological components. For example, a purified polypeptide or protein will often be sufficiently free of other protein components so that degradative sequencing may be performed successfully.

Various methods for quantifying the degree of purification of proteins and polypeptides comprising the amino acid sequence of SEQ ID NO:2 or SEQ ID NO:4 will be known to those of skill in the art in light of the present disclosure. These include, for example, assessing the number of polypeptides within a fraction by SDS/PAGE analysis will often be preferred in the context of the present invention as this is straightforward.

To purify a protein and polypeptide comprising the amino acid sequence of SEQ ID NO:2 or SEQ ID NO:4, a natural or recombinant composition comprising at least some protein and polypeptide comprising the amino acid sequence of SEQ ID NO:2 or SEQ ID NO:4 will be subjected to fractionation to remove various components not comprising the amino acid sequence of SEQ ID NO:2 or SEQ ID NO:4 from the composition. Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques.

Although preferred for use in certain embodiments, there is no general requirement that the protein and polypeptide comprising the amino acid sequence of SEQ ID NO:2 or SEQ ID NO:4 always be provided in their most purified state. Indeed, it is contemplated that less substantially purified proteins and polypeptides comprising the amino acid sequence of SEQ ID NO:2 or SEQ ID NO:4, which are nonetheless enriched in protein and polypeptide comprising the amino acid sequence of SEQ ID NO:2 or SEQ ID NO:4 compositions, relative to the natural state, will have utility in certain embodiments. These include, for example, antibody generation where subsequent screening assays using purified protein and polypeptide comprising the amino acid sequence of SEQ ID NO:1 or SEQ ID NO:4.

Methods exhibiting a lower degree of relative purification may have advantages in total recovery of protein product, or in maintaining the activity of an expressed protein. Inactive products also have utility in certain embodiments, such as, e.g., in antibody generation.

Modifications and changes can be made in the structure of a polypeptide of the present invention and still obtain a molecule having like characteristics and function. For example, certain amino acids can be substituted for other amino acids in a sequence without appreciable loss of structure or activity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with the like properties.

In making such changes, the hydropathic index of amino acids can be considered. The importance of the hydropathic amino acid index in conferring interactive biologic function on a polypeptide is generally understood in the art (Kyte and Doolittle, 1982). It is known that certain amino acids can be substituted for other amino acids having a similar hydropathic index or score and still result in a polypeptide with similar biological activity. Each amino acid has been assigned a hydropathic index based on its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−0.3); proline (−1.6); histidine (−3.2); glutamate (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines that interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +/−2 is preferred, those which are within +/−1 are particularly preferred, and those within +/1 0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated herein by reference, states that the greatest local average hydrophilicity of a polypeptide, as governed by the hydrophilicity of its adjacent amino acids, correlates with its immunogenicity and antigenicity, i.e. with a biological property of the polypeptide.

As detailed in U.S. Pat. No. 4,554,101, the following hydrophilicity values have been assigned to amino acid residues: arginine (+3.0); lysine (+3.0); aspartate (+3.0+/−1); glutamate (+3.0+/−1); serine (+0.3); asparagine (+0.2); glutamine (+0.2); glycine (0); proline (−0.5+/11); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within +/−2 is preferred, those which are with in +/−1 are particularly preferred, and those within +/=0.5 are even more particularly preferred.

As outlined above, amino acid substitution are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (see Table 2).

TABLE 2

| | Exemplary |
|---|---|
| Original Residue | Substitutions |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Met; Leu; Tyr |
| Ser | Thr |
| Thr | Ser |
| Trp | Tyr |
| Tyr | Trp; Phe |
| Val | Ile; Leu |

Mutagenesis

Site-specific mutagenesis is a technique useful in the preparation of individual polypeptides, or biologically functional equivalent proteins or polypeptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector that includes within it sequence a DNA sequence encoding the desired protein. An oligonucleotide primer bearing the desired mutated sequence is synthetically prepared. This primer is then annealed with the single-stranded DNA preparation, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli dut ung strains, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

Antibodies to Epitopic Core Sequences

Polypeptides corresponding to one or more antigenic determinants, or “epitopic core regions”, of a polypeptide comprising the amino acid sequence of SEQ ID NO:2 or SEQ ID NO:4 can also be prepared. Such polypeptides should generally be at least five or six amino acid residues in length, will preferably be about 10, 15, 20, 25 or about 30 amino acid residues in length, and may contain up to about 35-50 residues or so.

Synthetic polypeptides will generally be about 35 residues long, which is the approximate upper length limit of automated polypeptide synthesis machines, such as those available from Applied Biosystems (Foster City, Calif.). Longer polypeptides may also be prepared, e.g., by recombinant means.

U.S. Pat. No. 4,554,101, (Hopp) incorporated herein by reference, teaches the identification and preparation of epitopes from primary amino acid sequences on the basis of hydrophilicity. Through the methods disclosed in Hopp, one of skill in the art would be able to identify epitopes from within an amino acid sequence such as the sequence disclosed herein (SEQ ID NO:2 or SEQ ID NO:4).

Numerous scientific publications have also been devoted to the prediction of secondary structure, and to the identification of epitopes, from analyses of amino acid sequences (Chou and Fasman, 1974a, b; 1978a, b, 1979). Any of these may be used, if desired, to supplement the teachings of Hopp in U.S. Pat. No. 4,554,101.

In further embodiments, major antigenic determinants of a polypeptide may be identified by an empirical approach in which portion of the gene encoding the polypeptide are expressed in a recombinant host, and the resulting proteins tested for their ability to elicit an immune response. For example, PCR can be used to prepare a range of polypeptides lacking successively longer fragments of the C-terminus of the protein. The immunoactivity of each of these polypeptides is determined to identify those fragments or domains of the polypeptide that are immunodominant. Further studies in which only a small number of amino acids are removed at each iteration then allows the location of the antigenic determinants of the polypeptide to be more precisely determined.

Another method for determining the major antigentic determinants of a polypeptide is the SPOTs™ system (Genosys Biotechnologies, Inc., The Woodlands, Tex.). In this method, overlapping polypeptides are synthesized on a cellulose membrane, which following synthesis and deprotection, is screened using a polyclonal or monoclonal antibody. The antigenic determinants of the polypeptides which are initially identified can be further localized by performing subsequent syntheses of smaller polypeptides with larger overlaps, and by eventually replacing individual amino acids at each position along the immunoreactive polypeptide.

Once one or more such analyses are completed, polypeptides are prepared that contain at least the essential features of one or more antigenic determinants. The polypeptides are then employed in the generation of antisera against the polypeptide. Minigenes or gene fusions encoding these determinants can also be constructed and inserted into expression vectors by standard methods, for example, using PCR cloning methodology.

The use of such small polypeptides for vaccination typically requires conjugation of the polypeptide to an immunogenic carrier protein, such as hepatitis B surface antigen, keyhole limpet hemocyanin or bovine serum albumin. Methods for performing this conjugation are well known in the art.

Antibody Generation

In certain embodiments, the present invention provides antibodies that bind to, or are immunoreactive with, proteins and polypeptides comprising the amino acid sequence of SEQ ID NO:2 or SEQ ID NO:4. Thus, antibodies that bind to the protein product of the isolated nucleic acid sequence of SEQ ID NO:1 or SEQ ID NO:3 are provided. As detailed above, in addition to antibodies generated against the full length proteins, antibodies may also be generated in response to smaller constructs comprising epitopic core regions, including wild-type, polymorphic and mutant epitopes.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (MAbs) are recognized to have certain advantages, e.g., reproducibility and large-scale production, and their use is generally preferred. The invention thus provides monoclonal antibodies of the human murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art.

Means for preparing and characterizing antibodies are well known in the art (Harlow and Lane (ed.) Antibodies: A Laboratory Manual, Cold Springs Harbor Laboratory, N.Y., 1988; incorporated herein by reference).

The methods for generating monoclonal antibodies (MAbs) generally begin along the same lines as those for preparing polyclonal antibodies. Briefly, a polyclonal antibody is prepared by immunizing an animal with an immunogenic composition containing a protein and polypeptide comprising the amino acid sequence of SEQ IDS NO:2 or SEQ ID NO:4 in accordance with the present invention and collecting antisera from that immunized animal.

A wide range of animal species can be used for the production of antisera. Typically, the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobenzoyl-N-hydroxysuccinimide ester, carbodiimide and bisbiazotized benzidine.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion. MHC antigens may even be used.

Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to co-administer biological response modifiers (BRM), which have been shown to upregulate T cell immunity or downregulate suppressor cell activity. Such BRMs include, but are not limited to, Cimetidine (CM; 1200 mg/d) (Smith/Kline, PA); or low-dose Cyclophosphamide (CYP; 300 mg/m²) (Johnson/Mead, NJ) and cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous, and intraperitoneal). The production of polyclonal antibodies may be monitored by sampling blood of the immunized animal at various points following immunization.

A second, booster injection, may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a polypeptide bound to a solid matrix, or by using, e.g., protein A or protein G chromatography.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified Rot or Rlp protein, polypeptide, peptide or domain, be it a wild-type or mutant composition. The immunizing composition is administered in a manner effective to stimulate antibody producing cells.

The methods for generating antibodies (MAbs) generally begin along the same lines as those for preparing polyclonal antibodies. Rodents such as mice and rats are preferred animals, however, the use of rabbit, sheep, or frog cells is also possible. The use of rats may provide certain advantages (Goding, 1986, pp. 60-61), but mice is preferred, with the BALB/c mouse being most preferred as this is most routinely used and generally gives a higher percentage of stable fusions.

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes form the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag4 1, Sp210-Ag14, OF, NSO/U, MPC11-X45-GTG 1.7 and S194/5XXO Bul; for rats, one may use R210.RCY3, Y3-Ag 1.2.3, IR983F and 4B210; and U-266, GM1500-GRG2, LICR-LON-Hmy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described and those using polyethylene glycol (PEG), such as 37% (v/v) PEG, by Gefter et al. (1977). The use of electrically induced fusion methods is also appropriate (Goding, pp. 71-74, 1986).

Fusion procedures usually produce viably hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis as a source of nucleotides (HAT medium). Where azaserine is used, the media is supplemented with hypoxanthine.

In certain examples, the preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, a selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC of affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Alternatively, monoclonal antibody fragments encompassed by the present invention can be synthesized using an automated peptide synthesizer, or by expression of full-length gene or of gene fragment in E. coli.

Antibody Conjugates

The present invention further provides antibodies against proteins and polypeptides comprising the amino acid sequence of SEQ ID NO:2 or SEQ ID NO:4, generally of the monoclonal type, that are linked to one or more other agents to form an antibody conjugate. Any antibody of sufficient selectivity, specificity and affinity may be employed as the basis for an antibody conjugate. Such properties may be evaluated using conventional immunological screening methodology known to those of skill in the art.

Certain examples of antibody conjugates are those conjugates in which the antibody is linked to a detectable label. “Detectable labels” are compounds or elements that can be detected due to their specific functional properties, or chemical characteristics, the use of which allows the antibody to which they are attached to be detected and further quantified if desired. Another such example is the formation of a conjugate comprising an antibody linked to a cytotoxic or anticellular agent, as may be termed “immunotoxins”. In the context of the present invention, immunotoxins are generally less preferred.

Antibody conjugates are thus preferred for use as diagnostic agents. Antibody diagnostics generally fall within two classes, those for use in in vitro diagnostic protocols, generally known as “antibody-directed imaging”.

Many appropriate imaging agents are known in the art, as are methods for their attachment to antibodies (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the antibody (U.S. Pat. No. 4,472,509). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

Radioactively labeled monoclonal antibodies of the present invention may be produced according to well-known methods in the art. For instance, monoclonal antibodies can be iodinated by contact with sodium or potassium iodide and a chemical oxidizing agent such as sodium hypochlorite, or an enzymatic oxidizing agent, such as lactoperoxidase. Monoclonal antibodies according to the invention may be labeled with technetium-99m by ligand exchange process, for example, by reducing pertechnate with stannous solution, chelating the reduced technetium onto a Sephadex column and applying the antibody to this column or by direct labeling techniques, e.g., by incubating pertechnate, a reducing agent such as SNCI₂, a buffer solution such as sodium-potassium phthalate solution, and the antibody.

Intermediary functional groups which are often used to bind radioisotopes which exist as metallic ions to antibody are diethylenetriaminecpentaacetic acid (DTPA) and ethylene diaminetetracetic acid (EDTA).

Essentially any fluorescent label, including rhodamine, fluorescein isothiocyanate and renograhin, may be used to produce an antibody conjugate of the present invention.

The much preferred antibody conjugates of the present invention are those intended primarily for use in vitro, where the antibody is linked to a secondary binding ligand or to an enzyme (an enzyme tag) that will generate a colored product upon contact with a chromogenic substrate.

Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art and is described, for example, in U.S. Pat. Nos. 3,817,837; 3,850,752; 3,939,350; 3,996,345; 4,277,437; 4,275,149; and 4,366,241; each incorporated herein by reference.

Immunodetection Methods

In still further embodiments, the present invention concerns immunodetection methods for binding, purifying, removing, quantifying or otherwise generally detecting biological components such as components containing proteins and polypeptides comprising the amino acid sequence of SEQ ID NO:2 or SEQ ID NO:4. The proteins or polypeptides of the present invention may be employed to detect and purify antibodies prepared in accordance with the present invention, and antibodies prepared in accordance with present invention, may be employed to detect proteins and polypeptides comprising the amino acid sequence of SEQ ID NO:2 or SEQ ID NO:4. As described throughout the present application, the use of antibodies specific to proteins and polypeptides comprising the amino acid sequence of SEQ ID NO:2 or SEQ ID NO:4 is contemplated. The steps of various useful immunodetection methods have been described in the scientific literature, such as, e.g., Nakamura et al. (1987), incorporated herein by reference.

In general, the immunobinding methods include obtaining a sample suspected of containing a protein and polypeptide comprising the amino acid sequence of SEQ ID NO:2 or SEQ ID NO:4, and contacting the sample with a first antibody in accordance with the present invention, as the case may be, under conditions effective to allow the formation of immunocomplexes.

These methods include methods for purifying proteins and polypeptides comprising the amino acid sequence of SEQ ID NO:2 or SEQ ID NO:4, as may be employed in purifying proteins and polypeptides comprising the amino acid sequence of SEQ ID NO:2 or SEQ ID NO:4 from patients' samples or for purifying recombinantly expressed proteins and polypeptides comprising the amino acid sequence of SEQ ID NO:2 or SEQ ID NO:4. In these instances, the antibody removes the antigenic component form a sample. The antibody will preferably be linked to a solid support, such as in the form of a column matrix, and the sample suspected of containing the antigenic component will be applied to the immobilized antibody. The unwanted components will be washed from the column, leaving the antigen immunocomplexed to the immobilized antibody, which antigen is then collected by removing the proteins and polypeptides comprising the amino acid sequence of SEQ ID NO:2 or SEQ ID NO:4 from the column.

The immunobinding, or immunoreactive, methods also include methods for detecting or quantifying the amount of reactive component in a sample, which methods require the detection or quantification of any immune complexes formed during the binding process. Here, one would obtain a sample suspected of containing a protein and polypeptide comprising the amino acid sequence of SEQ ID NO:2 or SEQ ID NO:4, and contact the sample with an antibody against proteins and polypeptides comprising the amino acid sequence of SEQ ID NO:2 or SEQ ID NO:4, and then detect or quantify the amount of immune complexes formed under the specific conditions.

In terms of antigen detection, the biological sample analyzed may be any sample that is suspected of containing a protein and polypeptide comprising the amino acid sequence of SEQ ID NO:2 or SEQ ID NO:4, typically such samples would be suspected of containing S. aureus.

Contacting the chosen biological sample with the antibody under conditions effective and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any proteins and polypeptides comprising the amino acid sequence of SEQ ID NO:2 or SEQ ID NO:4 present. After this time, the sample-antibody composition, such as a tissue section, ELISA plate, dot blot or western blot, will generally be washed to remove any non-specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological or enzymatic tags. U.S. Patents concerning the use of such labels include U.S. Pat. Nos. 3,817,837; 3,850,752; 3,939,350; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The antibody specific to proteins and polypeptides comprising the amino acid sequence of SEQ ID NO:2 or SEQ ID NO:4 employed in the detection may itself be linked to a detectable label, wherein one would then simply detect this label, thereby allowing the amount of the primary immune complexes in the composition to be determined.

Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for th antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

The immunodetection methods of the present invention have evident utility in the diagnosis or prognosis of S. aureus infections, intoxications, and syndromes. Here, a biological or clinical sample suspected of containing a protein and polypeptide comprising the amino acid sequence of SEQ ID NO:2 or SEQ ID NO:4 is used. However, these embodiments also have applications to non-clinical samples, such as in the titering of antigen or antibody samples, in the selection of hybridomas, and the like.

Immunochemical methods include, but are not limited to, Western blotting, immunoaffinity purification, immunoprecipitation, ELISA, dot or slot blotting, RIA, immunohistochemical staining, immunocytochemical staining, and flow cytometry. Such techniques are well known to those of skill in the art.

Screening Assays

In yet another aspect, the present invention provides a process of screening substances for their ability to affect virulence factor expression in bacteria, particularly S. aureus. Utilizing the methods and compositions of the present invention, screening assays for the testing of candidate substances can be derived. A candidate substance is a substance that potentially can promote or inhibit virulence factor expression in bacteria.

A screening assay of the present invention generally involves determining the ability of a candidate substance to affect cellular processes leading to an alteration in expression of Rot or Rlp and detecting this alteration using nucleic acid or antibody compositions of the present invention. Alternatively, a screening assay of the present invention may involve determining the ability of a candidate substance to affect the biological activity of Rot or Rlp.

Rot and Rlp are described herein as proteins that affect the expression of virulence factors in S. aureus. Rot, as described herein, represses the expression of toxins in S. aureus. To screen for compounds that decrease toxin expression, one may contact an S. aureus culture with a candidate compound and monitor for increase expression or biological activity of Rot using the compositions and methods disclosed herein. To screen for compounds that increase toxin expression, one may contact an S. aureus culture with a candidate compound and monitor for decreased expression or biological activity of Rot using the compositions and methods disclosed herein.

Rlp, as described herein, induces expression of virulence factors in S. aureus. To screen for compounds that decrease toxin expression, one may contact an S. aureus culture with a candidate compound and monitor for decreased expression or biological activity of Rlp using the compositions and methods disclosed herein. To screen for compounds that increase toxin expression, one may contact an S. aureus culture with a candidate compound and monitor for increased expression or biological activity of Rlp using the compositions and methods disclosed herein.

The culture is exposed to the candidate compound under conditions and for a period of time sufficient for affecting virulence factor expression. Such conditions and time periods may be determined by using compounds known to affect virulence factor expression.

Exposure will vary inter alia with the biological conditions used, the concentration of compound and the nature of the culture. Means for determining exposure time are well known to one of ordinary skill in the art.

The candidate compound may be essentially any compound or composition suspected of being capable of affecting biological functions or interactions. The compound or composition may be part of a library of compounds or compositions. Alternatively, the compound or compositions may be designed specifically to interact or interfere with the biological activity of the compositions of the present invention, e.g., antisense constructs, double-stranded nucleic acid molecules containing one or more Rot or Rlp binding sites, or compositions containing or encoding dominant negative Rot or Rlp polypeptides (polypeptides with missing or alternative domains as compared to the wild-type protein).

Therapeutic Compositions and Methods

Since S. aureus can cause many different diseases, compositions that affect the virulence of S. aureus are i15 useful in methods of treating S. aureus-associated diseases and disorders. Such diseases and disorders include, but are not limited to, skin wounds and infections, tissue abscesses, folliculitis, food poisoning, osteomyelitis, pneumonia, scalded skin syndrome, septicemia, septic arthritis, myocarditis, endocarditis, and toxic shock syndrome. Furthermore, the compositions affecting virulence may be useful in preventive treatments (i.e., a patient that is thought to be a candidate for a S. aureus infection).

In certain aspects of the present invention, a composition that decreases virulence factor expression is administered to treat or prevent a S. aureus-associated disease and disorder. An example of a composition includes a composition comprising SEQ ID NO:1 or functional fragments and variations thereof. Another example is a composition comprising a compound that increases the expression of SEQ ID NO:1 in S. aureus. The compound may be a compound found by the screening assays of the present invention. Another example is a composition that suppresses the expression or activity of SEQ ID NO:3. Such composition may include a compound found by the screening assays of the present invention. Such compounds also include nucleic acids found to express molecules that have dominant negative activity over the activity of SEQ ID NO: 3.

Methods of administering compounds to treat S. aureus infections are well known to those of skill in the art. For example, methods of and pharmaceutical compositions for treating S. aureus infections are described in U.S. Pat. No. 5,846,772, incorporated herein by reference.

The pharmaceutical compositions may be administered in any effective, convenient manner including, for instance, administration by topical, oral, anal, vaginal, intravenous, intraperitoneal, intramuscular, subcutaneous, intranasal or intradermal routes among others.

The pharmaceutical compositions generally are administered in an amount effective for treatment of prophylaxis of a specific indication or indications. In general, the compositions are administered in an amount of at least about 10 μg/kg body weight. In most cases they will be administered in an amount not in excess of about 10 mg/kg body weight per day. Preferably, in most cases, dose is from about 10 μg/kg to about 1 mg/kg body weight, daily. It will be appreciated that optimum dosage will be determined by standard methods for each treatment modality and indication, taking into account the indication, its severity, route of administration, complicating conditions and the like.

In therapy or as a prophylactic, the active agent may be administered to an individual as an injectable composition, for example as a sterile aqueous dispersion, preferably isotonic.

Alternatively, the composition may be formulated for topical application, for example, in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

For administration to mammals, and particularly humans, it is expected that the daily dosage level of the active agent will be from 0.01 mg/kg to 10 mg/kg, typically around 1 mg/kg. The physician in any event will determine the actual dosage which will be most suitable for an individual and will vary with the age, weight and response of the particular case. There can, of course, be individual instances where higher or lower dosage ranges are merited, and such are within the scope of this invention.

Kits

In another aspect, the present invention provides for kits for detecting the presence of transcripts that encode Rot or Rlp or epitopes that are immunoreactive with the antibodies of the present invention.

Kits comprising antibodies may comprise a first container containing a first antibody being an antibody immunoreactive with proteins and polypeptides comprising the amino acid sequence of SEQ ID NO:2 or SEQ ID NO:4, with the antibody present in an amount sufficient to perform at least one assay. The assay kits of the invention may further comprise a second container containing a second antibody that immunoreacts with the first antibody. Preferably, the secondary antibody is conjugated with a label (enzymatic, fluorometric, radioactive, etc.). The secondary antibody may be from essentially any animal including, but not limited to cow, goat, sheep, horse, rabbit, chicken, or donkey.

### DETAILED DESCRIPTIONS OF THE FIGURES

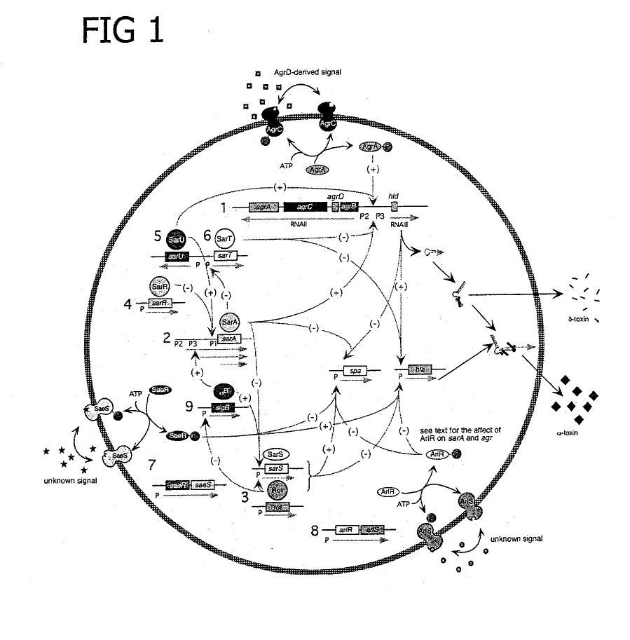

FIG. 1. A Model for the regulation of staphylococcal virulence factors. A single S. aureus is delineated by the large hatched circle. Genes are represented by boxes and promoters are labeled “P”. With the exception of RNAIII (arrow and ladder-like structure) and the α-toxin message (arrow), mRNA is depicted using straight lines with triangular arrowheads. 1. The P2 and P3 operons of the agr locus. The agr P2 promoter transcribes RNAII, which encodes AgrA, AgrC, AgrD, and AgrB. The agr P3 promoter transcribes RNAIII, which encodes 6-toxin (hld). AgrC is activated by autophosphorylation after binding the AgrD-derived peptide pheromone. The phosphate group (circle) is transferred from AgrC to AgrA. Activated AgrA functions to increase transcription RNAII and RNAIII. RNAIII is associated with the down-regulation of cell-surface protein genes and the up-regulation of extracellular protein genes including α-toxin (hla). RNAIII is required for translational control of the hla message. Translation of δ-toxin (short lines) is the only known protein product of RNAIII. 2. The sar locus is transcribed on three messages each encoding SarA. SarA is required for the activation of agr P2 promoter. SarA, the sar transcripts, or an uncharacterized protein encoded on the SarA messages is involved in the down-regulation of transcription of the gene encoding protein A (spa). 3. Rot and SarS are involved in the up-regulation of cell-surface protein gene transcription and the down-regulation of extracellular protein gene transcription. Rot represses transcription of sarS. 4. SarR represses transcription of sarA. 5. SarU is required for of sarA and agr transcription. 6. SarT is a repressor of agr and hla. 7. The sae locus encodes a phosphate-transferring two-component signal transduction system that affects both cell surface and extracellular protein expression by an unknown mechanism. 8. The arlRS locus encodes a phosphate-transferring two-component signal transduction system that down-regulates transcription of spa and hla. Not shown in this model are affects of components of arlRS that up-regulate transcription from the agr P2 and P3 promoters and down-regulate transcription on sarA. 9. The sarA P3 and a second sarS promoter are dependent upon σ^{B}. Rot down-regulates sB expression.

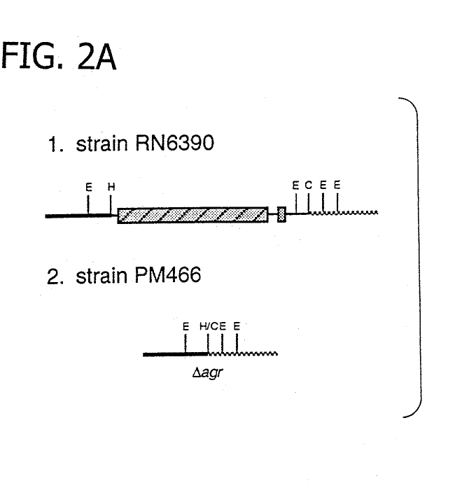

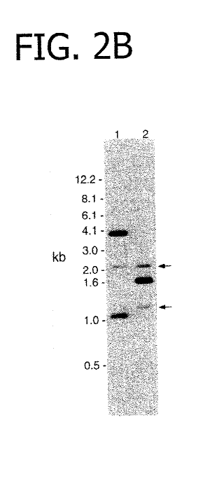

FIG. 2. (Panel A) An EcoRI(E) restriction map of the wild type allele of agr in RN6390 (top) and the agr-null allele (Δagr) in PM466 (bottom). The agr P2 operon and P3 transcript are represented by the striped and shade boxes, respectively. Chromosomal DNA on the 3′-end of the P2 operon and 3′-end of the P3 transcript are represented by a black and grey lines, respectively. (Panel B) Chromosomal DNA from RN6390 (lane 1) and PM466 (lane 2) digested with EcoRI and analyzed by Southern hybridization using agr and flanking ssDNA as the probe. DNA hybridizing to agr-flanking regions is marked with arrows. Abbreviations: C: ClaI; H: HincII; H/C: ligation of a HincII site with a T₄ polymerase blunt-ended ClaI site.

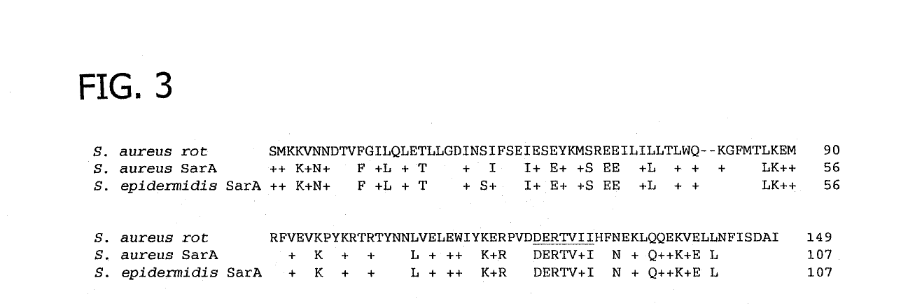

FIG. 3. BLASTP alignment of the rot gene product (Rot) and the S. aureus regulatory protein SarA, (e value 5.9) and the S. epidermidis SarA (e value=0.49). Rot was used as the query sequence against the non-redundant database at the National Center for Biotechnology Information. Identities are shown, (+) denotes similarity, and numbers at right refer to amino acids in the respective proteins.

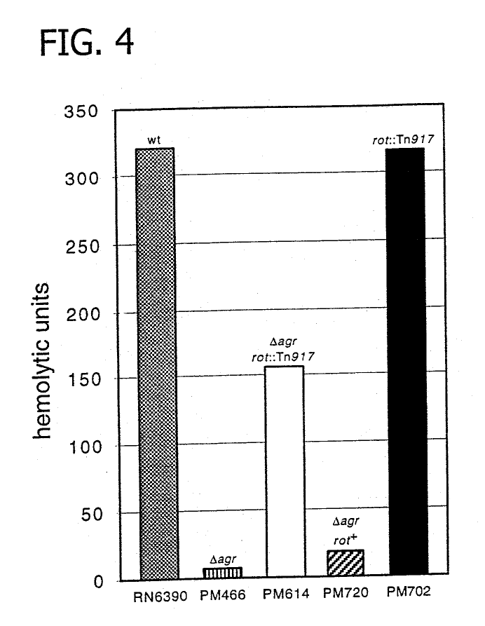

FIG. 4. Quantitative measurements of α-toxin activity in supernatant fluids from post-exponential phase (10 hour) cultures of S. aureus strains RN6390 (wild type, gray bar), PM466 (vertically striped bar), PM614 (white bar), PM720 (diagonal striped bar), and PM702 (black bar). Relevant genotypes are shown (wt: wild type; Δagr: agr-null allele, rot: wild type rot allele; rot::Tn917: insertionally inactivated rot).

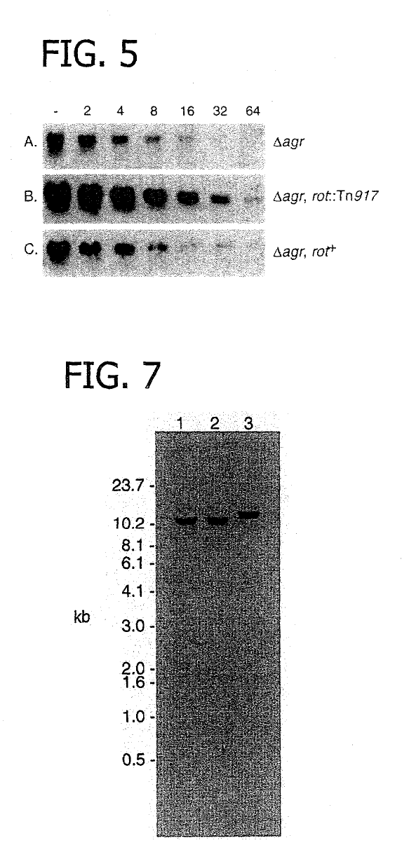

FIG. 5. Northern analysis of the α-toxin message in RNA from post-exponential phase cultures of S. aureus strains (Panel A) PM466 (Panel B) PM614, and (Panel C) PM720. Lane 1 contains 30 μg of total RNA serially diluted (1:2) in lanes 2-7. The transcript was identified by hybridization using digoxigenin-labeled probe specific for the gene encoding α-toxin with chemiluminescent chemistry.

FIG. 6. BLASTP alignment of the gene products of rlp and sarA. Identities are shown, (+) denotes similarity, and numbers at right refer to amino acids in the respective proteins.

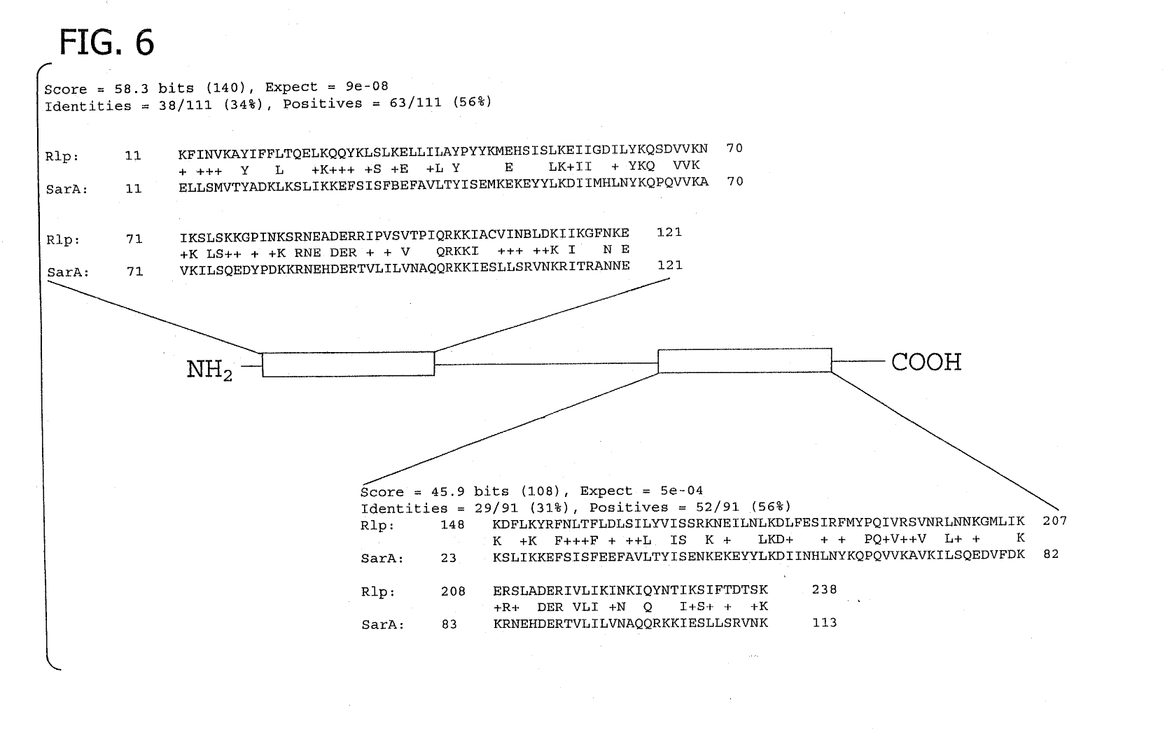

FIG. 7. Southern analysis of chromosomal DNA from S. aureus strains PM734, PM743, and RN6390. A 10 kb PstI fragment hybridized with labeled rlp in the wild-type strain RN6390 (lane 1). This is in contrast to the 12 kb fragment seen in the mutant strain, PM734, in which rlp is interrupted by a 2 kb erythromycin-encoding cassette (rlp::erm) that does not encode a PstI site (lane 3). Lane 2 contains PstI-digested chromosomal DNA from strain PM743, PM734 with a the rlp::erm allele restored to wild-type rlp by allelic exchange. rlp is now also known as sarU by agreement among the Staphylococcus aureus researchers.

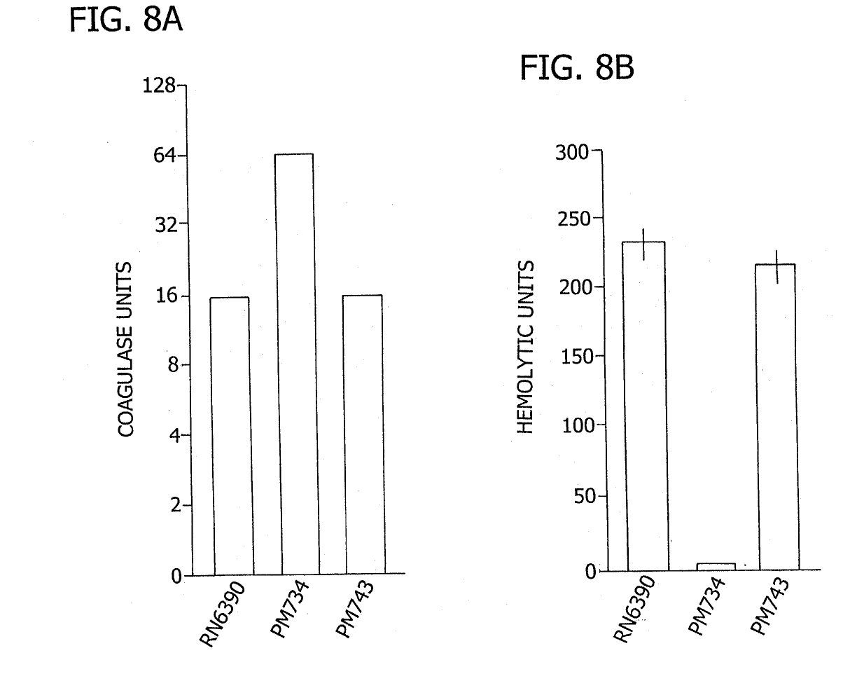

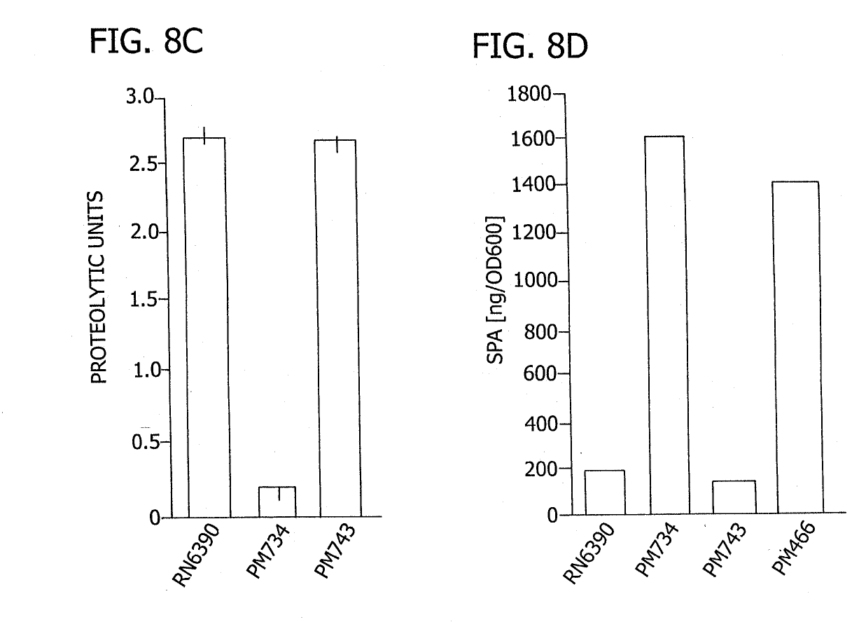

FIG. 8. Activity data from select extracellular and cell surface staphylococcal virulence-associated proteins. (Panel A) cell-surface coagulase, measured as reciprocal of the dilution of cultures standardized by optical density that formed a clot in rabbit plasma (Smeltzer et al., 1993); (Panel B) α-toxin, measured as the reciprocal of the dilution of culture supernatant fluids from a standard number of bacteria that yielded 50% lysis, (Panel C) total proteolytic activity measured azocasien hydrolysis (Smeltzer et al., 1993), and (Panel D) extracellular protein A measured by ELISA. Data from an agr-minus strain is included for reference. Activity and protein levels were measured in S. aureus strains RN6390 (wild-type), PM734 (sarU::ermC), PM743 (sarU-restored), and PM466 (Dagr).

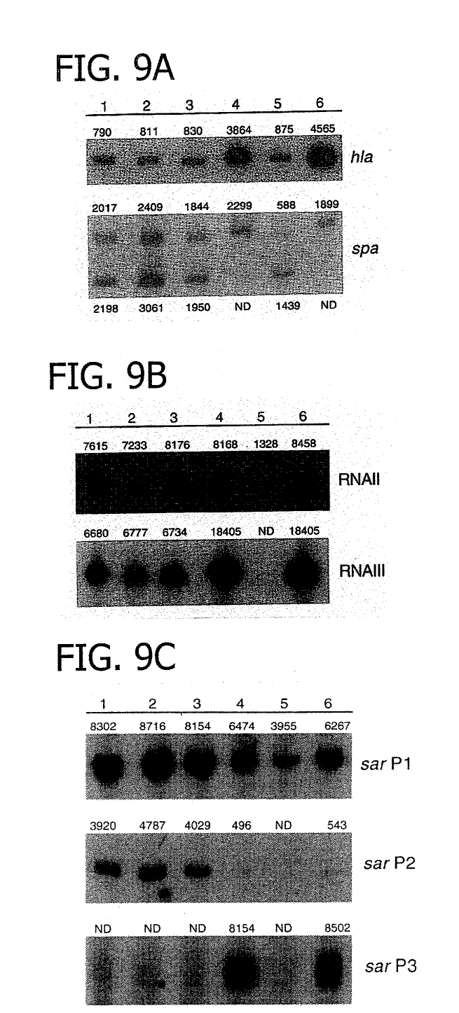

FIG. 9. Primer extension analysis of the α-toxin (hla) and protein A (spa) messages (panel A), RNAII and RNAIII (panel B), and sar (panel C), in RNA from exponential phase cultures (lanes 1-3 and 4-6, respectively) of S. aureus strains RN6390 (lanes 1 and 4), PM734 (lanes 2 and 5) and PM743 (lanes 3 and 6). Relative levels of specific messages calculated as the area in square pixels are given above each band with the exception of the smaller spa primer extension products which are listed below.

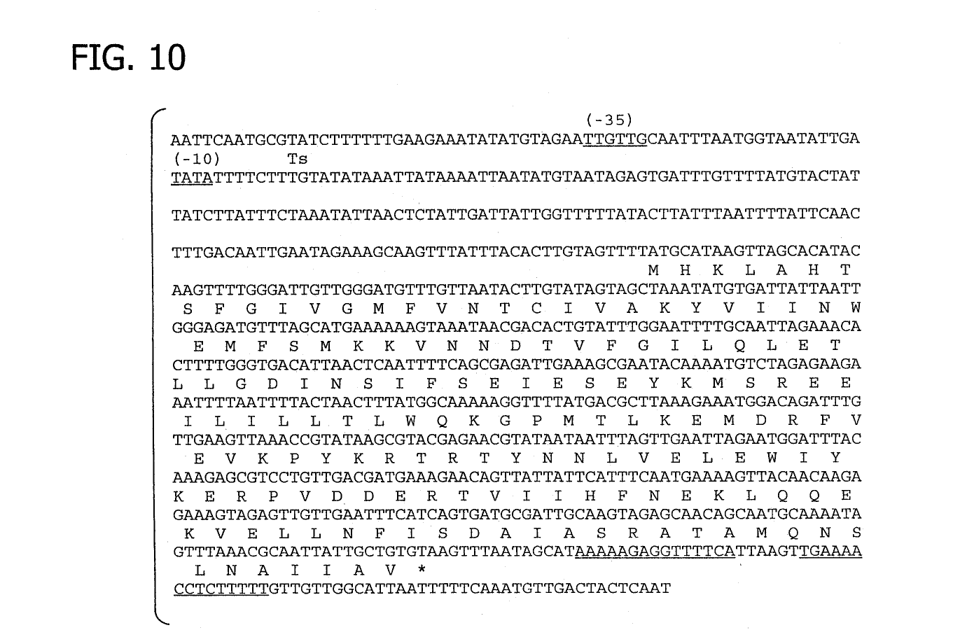

FIG. 10. Nucleic acid sequence (SEQ ID NO:12) containing the rot gene. The −35 and TATA box sequences are underlined and indicated by (−35) and (−10), respectively. Ts indicates the putative transcriptional start site. The Rot open-reading frame is indicated by segmenting the sequence into codons and the encoded amino acids are shown below their respective codons. The translational stop codon is indicated by *. Putative transcriptional termination sequences are underline in the 3′ region of the sequence.

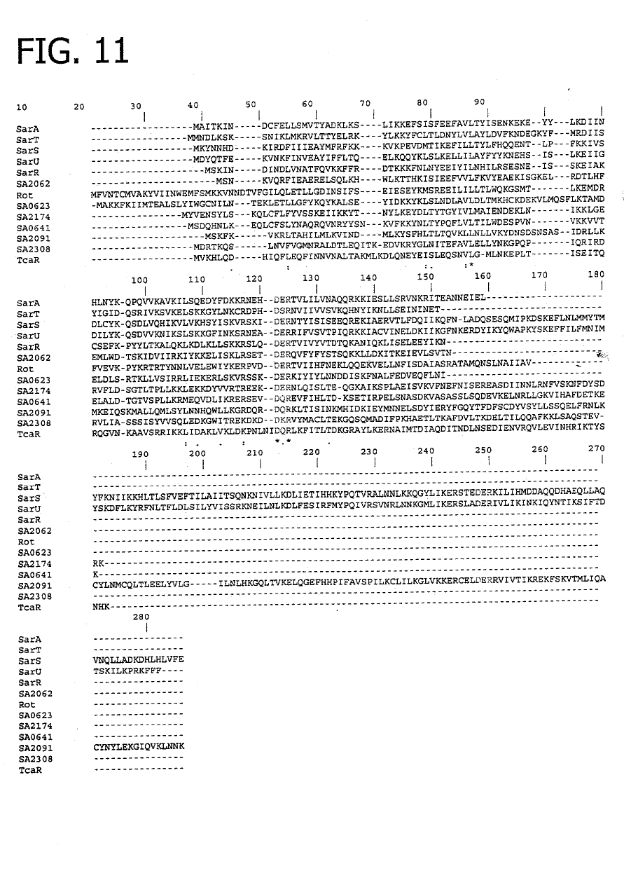

FIG. 11. A Clustal alignment of the amino acids, represented by the single letter code, of the S. aureus strain N315 Sar-homologues. The sequences of the characterized Sar-homologues are labeled. Uncharacterized Sar-homologues are labeled with the GenBank numbers assigned to proteins from strain N315. The degree of similarity of the amino acids in the proteins, from low to high, is displayed as periods, colons, and asterisks. Note that SarU, SarS, and SA2091 have two regions of homology with the other Sar-homologues. This region encompassed the “DER” sequence and has homology with the helix-turn-helix wing region of the putative DNA-binding domain of MarR (McNamara et al., 2000).

### EXAMPLES

The following examples are included to demonstrate embodiments of the invention. It should be appreciated by those of skill in the art that the techniques disclosed in the examples that follow represent techniques discovered by the inventors to function well in the practice of the invention, and thus can be considered to constitute preferred modes for its practice. However, those skilled in the art should, in light of the present disclosure, appreciate that many changes can be made in the specific embodiments that are disclosed and still obtain like or similar results without departing from the spirit and scope of the invention.

### Example 1

This example describes the isolation and characterization of a gene involved in the expression of virulence factors in S. aureus. The inventors have designated this gene rot, repressor of toxins.

Materials and Methods

Bacterial Strains, Phase, Plasmids, Media, Growth Conditions, and Virulence Factor Assays.

Bacteria, bacteriophage, and plasmids used in this example are described in Table 3.

TABLE 3
Summary of bacterial strains, bacteriophage, and plasmids

| Strain, phage, | | |
|---|---|---|
| or plasmid | Genotype, phenotype description | Reference or source |
| E. coli | | |
| DH5α | Fφ80lacZΔM15 Δ(lacZYA-argF)U196 and A1 recA1 | BRL |
| | hasdR17(rk⁻mk⁺)deoR thi-1 supE44 λ⁻ gyrA96 relA1 | |
| S. aureus | | |
| 8325-4 | wild type strain 8325 UV cured ofphagesφ11, φ12, and φ13 | NCTC |
| PM466 | agr-null mutant of RN6390 | This Example |
| PM614 | PM466 chr::Tn917::rot φ11 transductant | This Example |
| PM615 | PM466 chr::Tn917::rot φ11 transductant | This Example |
| PM616 | PM466 chr::Tn917::rot φ11 transductant | This Example |
| PM702 | RN6390 chr::Tn917::rot φ11 transductant | This Example |
| PM720 | PM614 with rot restored by allelic exchange | This Example |
| RN4220 | 8325-4, nitrosoguanidine-induced-restriction-minus mutant used | |
| | as primary recipient for plasmids propagated in E. coli | |
| RN6390 | 8325-4 | Novick et al. |
| Phage | | |
| φ11 | S. aureus generalized transducing phage φ | |
| Plasmids | | |
| pBC SK | cloning vector | Stratagene |
| pBCRII(+) | t-tail cloning vector | Invitrogen |
| pJM33 | pRN6650 with a 3.3-kb agr encoding ClaI-HpaIdeletion | This Example |
| pIM37 | pBluescript KS with a 1.2-kB HindIII insert that | This Example |
| | encodes the transposase of Tn 917 | |
| pJM48 | pSPT181(ts)::agr-null | This Example |
| pJM202 | pSPT181(ts)::rot | This Example |
| pJM531 | pCR-Script:hla; α-toxin gene from RN6390 amplified using primers | This Example |
| | 5′-GGAAGCTTAAACATCATTTCTGAAGTTATCGGC-3′ (SEQ ID NO:6) | |
| | and 5′-GGGACTAGTGAAGGATGATGAAAATGAAAACACG-3′ (SEQ ID NO:7) | |
| pRN6650 | pUC17:agr; contains a 6.1-kb MboI agr encoding fragment | Regassa et al., 1992 |
| pSPT181(ts) | temperature sensitive S. aureus-E. coli shuttle vector | Janzon and Arvidson, 1990 |
| pTV1 | E. coli-S. aureus shuttle vector with Tn917 | Youngman, 1987 |

S. aureus was cultivated in Trypticase Soy Broth (TSB, Difco Laboratories, Detroit, Mich.) and incubated at 37° C. with rotary agitation at 200 rpm or grown on Trypticase Soy agar plates (TSA). Escherichia coli was grown at 37 [symbol] C. in Luria-Bertani broth (LB) with agitation or on LB agar. Antibiotic-resistant staphylococci were selected and maintained at 10 μg ml⁻¹ tetracycline or 5 μg ml⁻¹ of either erythromycin or chloramphenicol. Resistant E. coli were grown in media augmented with 100 μg ml⁻¹ ampicillin. The method used for quantitative measurement of α-toxin has been previously described (Hart et al., 1993; McNamara and Iandolo, 1998). Assays for coagulase and protease have been described by Hart et al.(1993).

DNA Isolation.

Chromosomal DNA was isolated from S. aureus using the method of Dyer and Iandolo (1983). Staphylococcal plasmid DNA was purified using a Qiagen Plasmid Mini Kit (Chatsworth, Calif.). The plasmid isolation procedure was modified by incubating the cell suspension in P1 buffer containing 100 μg ml⁻¹ recombinant lysostaphin (AMBI UK, Trowbridge, UK) for 30 min at 37 [symbol] C. The procedure was further modified by removal of the precipitate formed after the addition of neutralization buffer by centrifugation for 30 min. Routine procedures were used to isolate DNA from E. coli (Ausubel et al., 1996).

Recombinant Techniques.

Plasmids were constructed and amplified in E. coli strain DH5α using standard recombinant DNA techniques (Ausubel et al., 1996). Restriction endonucleases, DNA modification enzymes, and polymerases were obtained from Promega BioTech (Madison, Wis.) and used as recommended by the manufacturer.

For staphylococcal transductions, bacteriophage φ11 lysates were obtained from infected strains grown in overlaid soft agar (TSB, 0.5 mM CaCl₂, 0.5% agar), sterilized by passage through 0.2 μm filters, and titered on S. aureus strain RN6390. Transductions consisted of 5×10¹¹ CFU ml⁻¹ of exponentially grown bacteria in TSB containing 0.5 mN CaCl₂ and 5×10¹⁰ PFU ml⁻¹ of bacteriophage in a total of 0.6 ml. After 5 min at room temperature, 1.5 ml of TSB containing 0.5 mM CaCl₂ was added and the tubes were incubated for 20 min at 37 [symbol] C. Following the addition of 1 ml of 0.2 mM sodium citrate, the cells were harvested by centrifugation at 4×10³× g for 20 min, resuspended in 1 ml of 0.2 mM sodium citrate, and plated on TSA supplemented with 2 mM sodium citrate and the appropriate antibiotic. Transductional frequencies, when reported, were based on scoring at least 65 colonies.

Transformation of S. aureus were conducted using the electrotransformation procedure of Kraemer and Iandolo (1990). All plasmid DNA initially isolated from E. coli were introduced into S. aureus RN4220 prior to introduction to other strains of S. aureus. Allelic exchange in S. aureus utilized pSPT181(ts)-based plasmids with the conditions for plasmid integration and co-integrate resolution have been described in detail by Janzon and Arvidson (Janzon and Arvidson, 1990).

Construction of S. aureus Strain PM466.

To create PM466, the agr locus was deleted from strain RN6390 by allelic exchange using plasmid pJM48. Plasmid pJM48 was constructed in multiple steps. Initially, a 3.3 kb ClaI-HpaI agr-encoding fragment was removed from plasmid pRN6650 creating pJM33. The 2.8 kb Eco-RIHindIII fragment from pJM33 that contains agr flanking DNA was then cloned into similar sites in pBC SK (Stratagene, La Jolla, Calif.). This fragment was removed from pBC SK by digestion with PstI and transferred into similar sites in the temperature sensitive shuttle vector pSPT181(ts), creating pJM48.

Transposon Tn917 Mutagenesis and Phenotypic Screens.

Strain PM466 was subjected to mutagenesis with transposon Tn917 carried on plasmid pTV1 (Youngman, 1987). To overcome the low transformation efficiency of S. aureus, a colony of PM466 harboring pTV1 was grown at 32 [symbol] C., the permissive temperature, on TSA containing chloramphenicol to create a pool of bacteria with pTV1. Mutant bacteria were selected at 42 [symbol] C. and screened for protease activity on Nutrient agar (Smibert and Krieg, 1981) with 5% Skim Milk (Difco Laboratories) and hemolytic activity on Blood Agar Base (Difco Laboratories) supplemented with 5% rabbit food.

Southern and Northern Hybridization.

Digested staphylococcal chromosomal DNA was subjected to electrophoresis through 0.7% agarose gels, transferred to nylon membranes (MagnaGraph, Fisher Scientific, Pittsburgh, Pa.), and probed using the Genius system (Boehringer Mannheim, Indianapolis, Ind.) as instructed by the manufacturer. Hybridizations used a randomly primed digoxigenin-labeled 6.1 kb BamHI fragment from pRN6650 that contains agr plus flanking DNA or a 1.2 kb HindIII probe from pIM36 that encodes the transposase of transposon Tn551, standard buffer plus 50% formamide for pre-hybridization and hybridization, and stringent washes performed at 68 [symbol] C. (McNamara and Iandolo, 1998). Detection used the chemiluminescent substrate disodium 3-(4-methoxyspiro{1,2-dioxetane-3,2′-(5′-chloro)tricyclo[3.3.2.2³⁷]decan}-4-yl) phenyl phosphate (CSPD, Boehringer Mannheim).

Total cellular RNA was isolated from 10 hour cultures of S. aureus by the method of Hart et al. (Hart et al., 1993) and purified using RNAeasy (Qiagen, Chatsworth, Calif.). Electrophoresis of RNA was conducted in 1% LE agarose glyoxal gels. The RNA was transferred to a nylon membrane (MagnaGraph), and probed with a ClaI-XbaI gragment from pIM42 that encodes part of RNAIII or a SpeI-HindIII fragment from plasmid pJM531 that encodes α-toxin. The probes were digoxigenin-labeled and hybridized using high SDS buffer at 50 [symbol] C. Stringent washes were performed at 65 [symbol] C. and detection CSPD. Levels of message were compared using Mutli-Analyst Version 1.02 software (Bio-Rad Laboratories, Hercules, Calif.).

Inverse-PCR and Nucleotide Sequencing.

Inverse-PCR reactions contained chromosomal DNA from strains PM614, PM615, and PM616 digested with either EcoRI or PstI and self-ligated at a concentration of 5 ng DNA μl⁻¹. The Tn917-specific outward facing primers were 5′-GAGCATATCCACTTTTCTTGGAG-3′ (SEQ ID NO:8) and 5′-CACAATAGAGAGATGTCACGTC-3′ (SEQ ID NO:9) (GenBank M11180). DNA was amplified by the method of Coen (Coen, 1992). The nucleotide sequence for rot was obtained using an Applied Biosystems 373A or 377 DNA Sequencer with dye terminator cycle sequening chemistry (Perkin Elmer, Foster City, Calif.) an Qiagen purified DNA (Chatsworth, Calif.). Template DNA consisted of a pool of three independently amplifed PCR products. Sequencing primers were designed to extend newly acquired sequence. Additional S. aureus sequence data was obtained from The Institute for Genomic Research (website at http://www.tigr.org). Data was analyzed usign the GCG Sequence Analysis Software Package Version 8.1 (Wu et al., 1996).

Construction of S. aureus Strain PM720.

PM720 was created by allelic exchange using S. aureus PM614 and plasmid pJM202. Plasmid pJM202 is plasmid pSPT181(ts) with a 1.3-kb PCR fragment generated from the wild type S. aureus strain RN6390 using primers that correspond to sequence upstream and downstream of rot, 5′-CAAAGCCTGACACGACAATCC-3′ (SEQ ID NO:10) and 5′-CTGAAAGATGAGACAGTAGATG-3′ (SEQ ID NO:11), respectively. To construct pJM202, the rot containing PCR fragment was cloned into plasmid pCR11(+)(Invitrogen, Carlsbad, Calif.) and verified by restriction endonuclease and sequence analysis. The rot fragment in PCR11 was removed by digestion with EcoRI and moved into a similar site within the multiple cloning site of pSPT181(ts).

Results

Construction of S. aureus Strain PM466.

Strain PM466 is a new agr-null derivative of S. aureus RN6390 created by allelic exchange using plasmid pJM48. The deletion in PM466 encompasses the entire agr P2 operon and the first 379 bp of the P3 transcript. The expected chromosomal deletion was confirmed in strain PM466 by Southern analysis (FIG. 2B). Measurements of virulence factor activity demonstrated that post-exponential phase culture supernatant fluids from PM466 had less than five percent of the protease and α-toxin activities associated with RN6390. Coagulase activity was approximately 10-fold higher in PM466 than the wild type control. RNAIII in PM466 could not be detected by Northern analysis.

Transposon Tn917 Mutagenesis and Transductional Analysis.

Strain PM466 was subjected to mutagenesis with transposon Tn917. Approximately 2×10⁴ bacterial with chromosomal insertions of the transposon were screened for proteolytic activity on Skim Milk agar both with and without erythromycin. Eleven protease-positive strains were isolated. To rule out mutations in genes that only activate protease expression, the erythromycin-resistant protease-positive strains were screened for hemolytic activity on rabbit blood agar plates. Nine of the original eleven isolates had α-toxin activity. The loss of plasmid pTV1 from these nine strains was confirmed by testing for vector-encoded antibiotic resistance on TSA supplemented with chloramphenicol at the minimal inhibitory concentration. The lack of growth of the nine strains in this media provides supporting evidence that the erythromycin resistance was mediated by a chromosomal insertion of the transposon.

To confirm the linkage between the transposon and the genetic lesion causing the restored phenotype, DNA surrounding the transposon from the presumptive mutant strains was back-transferred in the agr-minus strains PM466 or RN6911 by transduction using bacteriophage φ11. In independent experiments, the protease- and α-toxin-positive phenotype was shown to co-transfer with transposon-encoded erythromycin resistance in four of the nine isolates. No differences in phenotype were observed between mutations in the two agr-null genetic backgrounds. Transduction of the erythromycin resistance marker into PM466 resulted in the isolation of strains PM614, PM615, and PM616. In these experiments, greater than 98% of the transductants had a protease and α-toxin-positive phenotype. In the remaining strains, genetic linkage could not be verified.

Southern analysis of chromosomal DNA from PM614, PM615, and PM616 using a Tn917-derived probe suggested a single gene conferred the restored extracellular protein phenotype. Single digests of the chromosomal DNA using four different restriction endonucleases that do not cut within the Tn917 resulted in an identical pattern of hybridizing DNA fragments. These data suggest that the chromosomal insertion of the transposon in the three strains occurred within the same gene.

DNA surrounding the insertion site of the transposon from strains PM614, PM615, and PM616 was amplified by inverse-PCR and the nucleotide sequence of approximately 2 kb of DNA flanking the transposon insertion site was determined. The size of the inverse-PCR products was consistent with values predicted from Southern analysis of the protease- and α-toxin-positive transductants. With each of these strains, the probe-hybridizing EcoRI fragment was 9 kb and the inverse-PCR product, minus Tn917 DNA, was approximately 4 kb. Furthermore, in each of these strains, the nucleotide duplication that occurs upon the transposition of Tn917 was found.

Nucleotide sequence analysis of the inverse-PCR products indicated that the transposon insertion site in PM614 and PM616 was identical. In strain PM615, the transposon had inserted into a different site within the same gene. The open reading frame for the interrupted gene is 498 bp in length (SEQ ID NO:1) (GenBank AF189239). The predicted protein begins at a ATG translational start and terminates after 161 amino acid residues at an TAA stop (SEQ ID NO:2). Alternatively, protein initiation may occur from a number of downstream in-frame ATG starts resulting in a shorter protein.

A BLASTP search using a conceptional translation of the predicted 161 amino acid protein identified hypothetical proteins (GenBank U89914 and Swiss-protein P54182) and a region of homology to SarA from S. aureus and S. epidermidis (FIG. 3). The transposon inactivated gene was named rot (repressor of toxins) because loss of a wild type allele results in the restoration of protease and α-toxin activities to S. aureus PM466 and to reflect the fact that it has homology to known transcriptional regulators and acts as a repressor of toxin synthesis.

Verification and Initial Characterization of the Rot Mutation

As viewed on indicator plates, inactivation of rot restores a post-exponential phase protease- and α-toxin-positive phenotype to the agr-null strain of S. aureus PM466. To quantify the effect of the rot mutation on virulence factor production, α-toxin activity in culture supernatant fluids from strains RN6390, PM466, PM614, and PM720 was compared (FIG. 4). PM466, the agr-minus strain has approximately 4% of the activity associated with RN6390, its wild type parental strain. When compared to the activity seen with PM466, the rot mutation in PM614 results in a 25-fold increase in α-toxin activity. This level is approximately half that associated with a wild type strain. Similar results were seen with PM615.

Hemolytic activity in PM614 can be restored to PM466 levels by replacement of the chromosomal insertion of Tn917 with a wild type copy of rot. PM614 was subjected to allelic exchange using plasmid pJM202 resulted in the isolation of colonies that lacked protease and α-toxin on indicator plates. The genome of one resulting strain, PM720, was examined by Southern analysis. This strain both lacked DNA that hybridized with the transposase-encoding insert from plasmid pIM36 and had the expected 4 kb EcoRI rot-hybridizing fragment. Measurement of hemolytic activity in culture supernatant fluids from PM702 revealed that the 1.3 kb rot encoding fragment in pJM202 is sufficient to return PM466-like levels of activity to PM614 (FIG. 4).

Transposon encoded erythromycin resistance was transduced from PM614 into the wild-type strain of S. aureus, RN6390. In the resulting transductants, no difference in protease activity could be visualized on indicator plates. One transductant, strain PM720 was shown by Southern analysis to have the expected rot mutation. In this strain, α-toxin activity was similar to that seen in RN6390 (FIG. 4).

Post-exponential phase α-toxin message from strains PM466, PM614, and PM702 as quanitified by Northern analysis (FIG. 5). Consistent with our activity data, the α-toxin transcript in PM614 (rot-minus, agr-null mutant) was elevated 6-fold when compared to the message found in the agr-null parental strain. Furthermore, in strain PM702 (PM614 with a wild type copy of rot) the α-toxin message is returned to PM466 levels.

Discussion

This example identifies a locus in S. aureus that encodes a regulator of virulence factors. This locus was named rot because the gene product acts as a repressor of toxins. In an agr-null background, a mutation in rot increases the expression of protease and α-toxin in an agr-minus, rot-minus strain.

PM466 is a derivative of RN6390, the wild type strain used to define the molecular genetics of agr and sar (Cheung and Projan, 1994; Peng et al., 1988). In contrast to S. aureus RN6911, the published RN6390-derived agr-null mutant strain, PM466 has a specific deletion rather than an antibiotic marker and an accompanying deletion of unknown extent (Novick et al., 1995). Despite this genetic difference, quantitative measurements of cell surface and extracellular proteins demonstrate that the two agr-null strains have a common phenotype. This observation supports previous findings where the changes seen in RN6311 were interpreted as being solely due to the inactivation of agr.

Initially, transposon Tn917 mutants were screened for restored protease activity. Although the protein or proteins responsible for the zone of proteolysis surrounding single colonies of bacteria on Skim Milk agar have not been definitely identified, this activity has been shown to be RNAIII-dependent (Chien et al., 1999). Wild type strains produce clear zones of proteolysis on indicator plates, while the agr-null strains RN6911 and PM466 lack this activity. To rule out mutations in genes that only up-regulate protease expression in the agr-minus genetic background, the protease-positive strains were screened for hemolytic activity. S. aureus produces four different hemolysins (α-, β-, δ- and γ-toxins); however, rabbit erythrocytes suspended in agar are only susceptible to the action of α- and δ-toxin, the hemolytic activity associated with mutants created in the PM466-background is due to α-toxin (Peng et al., 1988). Despite the fact that RNAIII has been reported to be required for α-toxin translation, several of the proteolytic mutants displayed a hemolytic phenotype (Morfeldt et al., 1995). Co-transductional analysis of the proteolytic- and α-toxin-positive mutants was used to verify genetic linkage between the extracellular protein phenotype and the erythromycin resistance encoded by the transposon. Finally, the phenotype associated with the rot-minus allele in the agr-minus strains was confirmed by demonstrating that wild type rot is sufficient to restore and agr-minus phenotype to PM614.

Quantitative measurements of α-toxin activity and Northern analysis of the corresponding message were used to verify rot and in part, define its activity. Measurements of α-toxin activity indicated that restoration of rot in PM614 completely represses α-toxin to agr-minus levels. Moreover, rot mutations were found to only partially restore α-toxin activity and message in PM614 demonstrating that regulation occurs at the level of transcription. Therefore, it is possible that the rot-encoded protein may up-regulate an activator that is necessary for full α-toxin expression.

This example demonstrates that rot encodes a repressor of extracellular virulence factor transcription. Although the inventor does not wish to be held to any specific mechanism of action, one model (FIG. 1) predicts that the rot gene product (Rot) binds within the promoter region of regulated genes during the lag and exponential phase of bacterial growth blocking their transcription. Transcription of Rot-regulated promoters occurs when levels of the bound repressor are decreased, thus exposing the promoter and allowing for the binding of transcriptional activators and RNA polymerase. This model is analogous to the H-NS/DsrA-RNA pathway of Escherichia coli (Sledjeski et al., 1996). In the E. coli system, DrsA-RNA is part of a complex that binds the histone-like protein (H-NS), thus relieving DNA secondary structure that inhibits the transcription of regulated genes (Lease et al., 1998). A competing hypothesis is that rot and agr may be components of independent, yet partially redundant, pathways. Under this scenario, the rot translation product may act as either a repressor or an activator of factors necessary for virulence factor synthesis. In either case, the rot-associated activity appears to be altered by an agr product or factors that are regulated by agr because the rot mutation does not alter α-toxin expression found in culture supernatant fluids from stationary cultures of wild type strains.

### Example 2

This example describes an erythromycin insertional knockout mutant of a new regulator in S. aureus, and the effect of the knockout on coagulase and α-toxin activities and messages. Additionally, it describes the examination of total extracellular protease activity and the effect of the mutation on the levels of RNAIII and the SarA messages.

Identification of rlp

BLASTP searches of the partially completed DNA sequence from S. aureus COL and GenBank genome, using the predicted rot gene product of Example 1, identified the staphylococcal regulator SarA, as well as previously uncharacterized gene encoding a potential 247 amino acid protein named herein rlp for rot-like protein (GenBank accession number AF288788). Rlp and SarA share approximately 34% identity and 56% homology over the range of reported amino acids (FIG. 6). The rlp locus was amplified by PCR and cloned to create plasmid pJM730 (Table 4).

Construction of S. aureus Strains PM734 and PM743

S. aureus strain PM734 was created by allelic exchange using plasmid pJM730. This plasmid has rlp interrupted by an erythromycin cassette (rlp::erm) cloned into the temperature sensitive shuttle vector pSPT181(ts). While the vector alone was incapable of integration into the staphylococcal chromosome, pJM730 was capable of coverting S. aureus strain RN6390 from a proteolytic and hemolytic phenotype to a non-proteolytic and non-hemolytic phenotype. Furthermore, 6% of these strains were erythromycin susceptible suggesting that the erythromycin gene was lost from the chromosome. The genetic lesion in one erythromycin-resistant mutant strain, PM734, was confirmed by Southern analysis (FIG. 7).

The Southern data could be verified by demonstrating that primers flanking rlp that amplify a 1.3 kb DNA fragment in strain RN6390 can be used to amplify a 3.3 kb DNA fragment in PM734. Digestion of the 3.3 kb PCR product with Csp45, the restriction enzyme used in the construction of the rlp::erm allele, resulted in three DNA fragments that correspond in size to the erythromycin cassette and flanking DNA fragments.

To demonstrate a genetic linkage between the rlp::erm mutation and the observed extracellular toxin deficiencies, the erythromycin resistance marker in PM734 was transduced to RN6390, and the resulting strains were screened for proteolytic and α-toxin activities. Back-transduction of the marker resulted in 100% of the transductants having the phenotype of its original strain.

Like PM734, the rlp-restored strain PM743 was generated by allelic exchange. When introduced into strain PM734 and grown under conditions that allowed for allelic exchange, plasmid PJM744 (wild-type rlp cloned into pSPT181(ts) could restore the proteolytic and hemolytic phenotype of the host strain. Again, several strains that acquired the wild-type phenotype were erythromycin susceptible suggesting that rlp::erm was lost from the chromosome. Southern analysis of one such isolate, PM743, demonstrated that allelic exchange resulted in the return of a wild-type genotype (FIG. 7).

α-Toxin, Protease, and Coagulase Activity in S. aureus Strains RN6390, PM734 and PM743

S. aureus RN6390, PM734 and PM743 were characterized by examining coagulase activity in whole cell cultures and α-toxin and protease activities in supernatant fluid from post-exponential phase bacteria (FIG. 8). The measured enzymatic activities demonstrated that the rlp::erm mutation in strain PM734 increased the expression of cell-associated coagulase and decreases the expression of extracellular α-toxin and protease activities when compared to the wild-type levels associated with strain RN6390. In PM734, the coagulase activity in exponential phase cultures was similar to that seen with RN6390, while post-exponential phase coagulase activity in PM734 was quadrupled that seen in RN6390. The expression of the extracellular proteins in post-exponential phase culture supernatant fluids from PM734 was reduced approximately 95-fold. Moreover, restoration of a wild-type locus at the site of the rlp::erm mutation returned α-toxin, protease, and coagulase activities to wild-type levels in strain PM743.

Primer Extension Analysis of α-Toxin, spa, rnaii, rnaiii, and sar Messages in S. aureus Strains RN6390, PM734, PM743, and PM466.

Total RNA from strains RN6390, PM734, PM743, and PM466 isolated during either exponential and post-exponential phase of growth was analyzed for, α-toxin, protein A, rnaii, rnaiii, and SarA messages by primer extension. The results from the exponential phase cultures are shown in FIGS. 9A, 9B, and 9C.

Discussion

An erythromycin knockout mutation of rlp (rlp::erm) was created by allelic exchange in the wild-type strain of S. aureus RN6390, a strain used in the study of staphylococcal regulators (Cheung and Projan, 1994; McNamara et al., 2000; Peng et al., 1988). Following allelic exchange, as visualized on indicator plates, the toxin-producing wild-type strain converted to a non-proteolytic and non-hemolytic phenotype. Southern analysis demonstrated that rlp-hybridizing chromosomal DNA from one altered strain, PM734, increased by 2 kb, the size of the inserted erythromycin gene (FIG. 7). The site of insertion was further confirmed by Csp45 digestion of a PCR-generated DNA fragment flanking the insertion site of the antibiotic cassette. In contrast to the data obtained form PM734, allelic exchange using the agr-null mutant strain PM466 as the host strain failed to visibly alter the proteolytic and hemolytic activities associated with RN6390. This finding demonstrates that rlp functions as a positive regulator, rather than a repressor of gene expression that confers the tested activities.

Transduction of the erythromycin resistance from strain PM734 to RN6390 confirmed 100% genetic linkage between the extracellular protein deficient phenotype of PM734 and the rlp:erm allele. Additionally, allelic exchange was demonstrated that a 1.2 kb rlp-encoding DNA fragment is sufficient to restore proteolytic and hemolytic activities to PM734. Southern analysis is one resulting strain, PM743, showed that this strain was genetically, as well as phenotypically, similar to the parent strain of the rlp::erm mutant. Collectively, these data strongly support that the knockout mutation is solely responsible for the aberrant phenotype of strain PM734.

A comparison of the activity of cell surface coagulase and extracellular α-toxin and protease production in the tested strains are supporting evidence that the rlp::erm mutation confers and agr-mutant phenotype S. aureus PM734 (Novick et al., 1995). As previously reported for agr-mutant strains, select cell surface proteins are expressed in the exponential phase, but not during post-exponential phase growth. In contrast, select extracellular proteins are only produced after the exponential growth phase. The reciprocal manner of protein expression can be inferred from the phenotype of the rlp::erm mutant strain with regard to coagulase, α-toxin, and protease activities.

Levels of coagulase and α-toxin messages mirrored the activity data providing evidence that the rlp gene product affects the transcription of virulence factor genes. A comparison of RNAIII levels in strains RN6390 (wild-type) and PM734 (rlp::erm) was made to distinguish between an agr-dependent or independent regulation. Unlike in RN6390, RNAIII could not be detected in RNA isolated from post-exponential phase cultures of PM734. These data provide supporting evidence that the rlp, like sar, encodes an activator of agr or is required for the transcription of sarA. To distinguish between these possibilities, the levels of sarA messages in RN6390 and PM734 were compared and found to be identical, providing support that the rlp gene products acts directly upon the agr promoters and that the phenotypic effect of the rlp::erm mutation is mediated by RNAIII.

Experimental Procedures

Bacterial Strains, Plasmids, and Growth Media and Conditions

Bacteria, bacteriophage, and plasmids used in this example are listed and described in Table 4. S. aureus strains were cultivated in Trypticase Soy Broth (TSB, Difco Laboratories, Detroit, Mich.) and incubated at 37 [symbol] C. In S. aureus, tetracycline selection used media supplemented with antibiotic at a concentration of 10 μg ml⁻¹. S. aureus with erythromycin resistance derived from plasmid pHB210 were grown in media with 30 ng ml⁻¹ of the antibiotic for 1 h prior to selection at a final antibiotic concentration of 5 μg ml⁻¹. Resistant E. coli were grown in media with 100 μg ampicillin ml⁻¹.

TABLE 4
Summary of bacterial strains, bacteriophage, and plasmids

| Strain, phage, | Genotype, phenotype, | Reference |
|---|---|---|
| or plasmid | description | or source |
| E. coli | | |
| DH5α | Fφ80lacZΔM15Δ(lacZyA-agrF)U196 | BRL |
| | endAI recA1 HsdR17(rk⁻mk⁺)deoR | |
| | thi-1 supE44 λgyrA96 relA1 | |
| TOP10 | F-,mcrAΔ(mrr-hsdRMS-mcrBC) | Invitrogen |
| | φ80lacZΔM15 ΔlacX74 deoR recA1 | |
| | araΔ139 Δ(ara-leu)7697 | |
| | ga1U galK rps(Strr) endA1 nupG | |
| S. aureus | | |
| 8325 | wild-type strain | NCTC |
| PM466 | RN6390Δagr | McNamara et |
| | | al., 2000 |
| PM734 | RN6390 rlp::erm | This Example |
| PM743 | PM734 rlp | This Example |
| RN6390 | 8325-4, nitrosoguanidine-induced | |
| | restriction-minus mutant used as | |
| | primary recipient for plasmids | |
| | propagated in E. coli | |
| RN6390 | 8325 UV cured of phage and plasmids | Novick et al., |
| | | 1993 |
| Phage | | |
| φ11 | S. aureus generalized transducing | |
| | phage | |
| Plasmids | | |
| pCR11(+) | t-tail cloning vector | Invitrogen |
| pHB201 | 6593 bp shuttle plasmid with | Bacillus |
| | cat, erm, ori-pBR322, ori-1060, | Genetic |
| | palT, rep-1060, cat86::lacZa | Stock Center |
| pIM42 | pT7Blue(R)::RNAIII | McNamara et |
| | | al., 1999 |
| pJM718 | pCRII(+)::rlp | This Example |
| pJM730 | pSPT181(ts)::rlp::erm | This Example |
| pJM744 | pSPT181(ts)::rlp | This Example |
| pJM202 | pSPT181(ts)::rlp::erm | This Example |
| pJM531 | pCR-Script::hla | McNamara et |
| | | al., 2000 |
| pSPT181(ts) | temperature sensitive S. aureus-E. coli | Janzon and |
| | shuttle vector | Arvidson, 1990 |

Phenotypic and Quantitative Activity Assays

Mutant bacteria were screened for protease activity on Nutient agar (Smibert et al., 1981) with 5% Skim Milk (Difco Laboratories) and hemolytic activity on Blood Agar Base (Difco Laboratories) supplemented with 5% rabbit blood. The methods used for quantitative measurement of α-toxin and protease have been previously described (McNamara et al., 2000; Hart et al., 1993). Coagulase assays used serial doubling dilutions of whole-cell staphylococcal cultures incubated with rabbit plasma (Difco Laboratories) as described by the manufacturer. Coagulase units were reported as the reciprocal of the greatest culture dilution that resulted in a solid clot within the assay tube.

Recombinant Techniques

Plasmids were constructed and amplified using either E. coli strain DH5-α or strain XL1-blue as the host with standard recombinant DNA techniques. Restriction endonucleases, DNA modification enzymes, and polymerases were obtained from Promega BioTech (Madison, Wis.) or Amersham Pharmacia Biotech (Piscataway, N.J.) and used as recommended by the manufacturer. S. aureus were transformed using the electroporation procedure of Kraemer and Iandolo (1990). All plasmid DNA initially isolated from E. coli were introduced into S. aureus strain RN4220 prior to introduction to S. aureus strains RN6390 or PM734. Staphylococcal transductions used bacteriophage ll (McNamara et al., 2000).

Allelic Exchange

Plasmid pJM730, the rlp knockout vector, was constructed in steps. Initially, rlp was amplified from S. aureus RN6390 by PCR, cloned into pCR11, and transformed into E. coli strain XL1-Blue. PCR was performed as described by Coen (1992) using primers 5′-GGATCCGCCCATGAAACTTTCCATCTG-3′ (SEQ ID NO:13) and 5′-GGATCCGCGAACGTTATGACGTTGGAG-3′ (SEQ ID NO:14). BamHI sites added to 5′-ends of primers are underlined. A gene coding inducible erythromycin resistance (erm) was PCR amplified from plasmid pHB201 using primers 5′-TTCGAATCGTGCGCTCTCCTGTTCC-3′ (SEQ ID NO:15) and 5′-TTCGAATGGCTTATTGGCATCCTGGC-3′ (SEQ ID NO:16). Csp 45 sites added to 5′-end of primers are underlined. Amplified erm was ligated into plasmid pCR11 and used to transform competent E. coli strain TOP10. Cloned erm was used to interrupt rlp at a unique Csp45 site within the open reading frame of rlp. Finally, the rlp::erm fragment was introduced in to the BamHI site of pSPT181(ts) generating plasmid pJM744. To create S. aureus strain PM734, plasmid pJM730 was used for allelic exchange in strain RN6390 with the conditions for plasmid integration and resolution that have been previously described by Janzon and Arvidson (1990). Mutant S. aureus were screened for loss of proteolytic and hemolytic activity on skim milk and rabbit blood agar plates, respectively. Erythromycin resistant bacteria with an altered phenotype were screened for the loss of pSPT181(ts) sequences on TS containing tetracycline. S. aureus strain PM743 was created in the same manner as strain PM734 using PM734 as the host with plasmid pJM744. Plasmid pJM744 is pSPT181(ts) with the rlp-encoding BamHI fragment from plasmid pJM718.

Southern and Primer Extension

Digested chromosomal DNA from S. aureus was subjected to electrophoresis through 0.7% agarose gels, transferred to nylon membranes (MagnaGraph, Fisher Scientific, Pittsburgh, Pa.), and probed using the KPL system (KPL, Gaithersberg, Md.) as instructed by the manufacturer. Hybridizations used a biotin-N₄-dCTP-labeled 1.3-kb DNA insert from plasmid pJM718 that encompasses rlp. Detection used the chemiluminescent substrate disodium 2-chloro-5-(4-methoxyspiro(1,2-dioxetane-3,2-(5′-chlooro)-tricyclo [3.3.1.13,7]decan)-4-yl)-1-phenyl phosphate (CSP-Star, Tropix, Inc., Foster City, Calif.).

Total cellular RNA was isolated from 3 and 10 hour cultures (OD₅₄₀ of approximately 0.2 and 3.0, respectively) of S. aureus and purified, as described by McNamara et al. (2000). Gene-specific primers for the genes encoding protein A (spa), α-toxin (hla), RNAII, and RNAIII were 5′-CCTAAAGTTACAGATGCAATACC-3′ (SEQ ID NO:17), 5′-CGAGGGTTAGTCAAAGTTG-3′ (SEQ ID NO:18), and 5′-GTGCCATTGAAATCACTCCTT-3′ (SEQ ID NO:19), respectively. The SarA primers have been previously described (Bayer et al, 1996). Primers were end-labeled with γ-³²P ATP using T4 polynucleotide kinase (Promega BioTech, Madison, Wis.) as described by the manufacturer. Complementary DNA was synthesized using 200 U Superscript II (Gibco BRL, Grand Island, N.Y.) in reactions with 1× First Strand Buffer, 0.01 M DTT, 0.05 μg/μl Actinomycin D, 0.1 mM dNTPs. The concentration of total RNA in reactions with hla-, RNAII-, and RNAIII-specific primers was 5 ug/ml. A total of 15 ug/ml total RNA was used with the sar- and spa-specific primers. Reaction mixtures were incubated at 50 [symbol] C. for 50 min. DNA sequence was determined for the promoter regions of the genes encoding protein A, α-toxin, RNAII, and RNAIII, using Thermo Sequenase (USE Corporation, Cleveland, Ohio) chemistry on pJM764, pJM765, and pJM440, respectively. Due to the intensity of the signals derived from the RNAIII primer extension products, these samples were diluted 1:15 prior to loading on the gel. Samples were subjected to electrophoresis through 6% polyacrylamide gels in Glycerol Tolerant Buffer (0.1 M Tris Base, 28 mM taurine, 0.5 mM Na₂EDTA. Intensities of bands were determined from scanned gels using a NIH Image.

### Example 3

This example describe the nucleic acid sequence (SEQ ID NO:5) of the genetic locus encoding Rot (GenBank AF189239). The open reading frame of Rot is underlined. FIG. 10 describes an annotated segment of this sequence.

CAGTAGATGCTCATCTTTTTTTAGAACTTTTTTAAGGTTGAAAATGTATA
TCACATTTTATACACATTTGATTTGTAAGAATGTTTTGATTTATACAAAT
CATATCTTGAAAAATAACCAATTTAGCCTCATTCGGTTTGATTTAATTTG
TTAAATTTAAGGCTAACTAATTAATTTAGTTTAATCAATTTTCATGAGAG
TTATATGTAATAAAAATTCAATGCGTATCTTTTTTGAAGAAATATATGTA
GAATTGTTGCAATTTAATGGTAATATTGATATATTTTCTTTGTATATAAA
TTATAAAATTAATATGTAATAGAGTGATTTGTTTTATGTACTATTATCTT
ATTTCTAAATATTAACTCTATTGATTATTGGTTTTTATACTTATTTAATT
TTATTCAACTTTGACAATTGAATAGAAAGCAAGTTTATTTACACTTGTAG
TTTTATGCATAAGTTAGCACATACAAGTTTTGCATTGTTGGGATGTTTGT
TAATACTTGTATAGTAGCTAAATATGTGATTATTAATTGGGAGATGTTTA
GCATGAAAAAAGTAAATAACGACACTGTATTTGGAATTTTGCAATTAGAA
ACACTTTTGGGTGACATTAACTCAATTTTCAGCGAGATTGAAAGCGAATA
CAAAATGTCTAGAGAAGAAATTTTAATTTTACTAACTTTATGGCAAAAAG
GTTTTATGACGCTTAAAGAAATGGACAGATTTGTTGAAGTTAAACCGTAT
AAGCGTACGAGAACGTATAATAATTTAGTTGAATTAGAATGGATTTACAA
AGAGCGTCCTGTTGACGATGAAAGAACAGTTATTATTCATTTCAATGAAA
AGTTACAACAAGAGAAAGTAGAGTTGTTGAATTTCATCAGTGATGCGATT
GCAAGTAGAGCAACAGCAATGCAAAATAGTTTAAACGCAATTATTGCTGT
GTAAGTTTAATAGCATAAAAAGAGGTTTTCATTAAGTTGAAAACCTCTTT
TTGTTGTTGGCATTAATTTTTCAAATGTTGACTACTCAATCCTAAATTAT
AAATAGTATAGCGCAGCAAATGCTTAAGAAATTTTTTCTATGGCACAAAT
GAATGGAGCATGATTACGTTGGTTTAAAAATTGATATTGCAAAACTTGCG
CATGCTTTTGATCCAAAGTACTCAAGTAATCAAGCAATGCATGCTTCTCA
ATTTGTCCTTCGCTATGACCATGATATATAACAAGTACAATAATACCTTC
AATTGACATTAATGATAGCAATGAATTAATAGCTTGGATTGTCGTGTCAG
GCTTTG

### Example 4

This example describes the nucleic acid sequence (SEQ ID NO:1) of an open reading frame encoding Rot.

ATGCATAAGTTAGCACATACAAGTTTTGGGATTGTTGGGATGTTTGTTAA
TACTTGTATAGTAGCTAAATATGTGATTATTAATTGGGAGATGTTTAGCA
TGAAAAAAGTAAATAACGACACTGTATTTGGAATTTTGCAATTAGAAACA
CTTTTGGGTGACATTAACTCAATTTTCAGCGAGATTGAAAGCGAATACAA
AATGTCTAGAGAAGAAATTTTAATTTTACTAACTTTATGGCAAAAAGGTT
TTATGACGCTTAAAGAAATGGACAGATTTGTTGAAGTTAAACCGTATAAG
CGTACGAGAACGTATAATAATTTAGTTGAATTAGAATGGATTTACAAAGA
GCGTCCTGTTGACGATGAAAGAACAGTTATTATTCATTTCAATGAAAAGT
TACAACAAGAGAAAGTAGAGTTGTTGAATTTCATCAGTGATGCGATTGCA
AGTAGAGCAACAGCAATGCAAAATAGTTTAAACGCAATTATTGCTGTGTAA

### Example 5

This example describes the amino acid sequence of Rot (SEQ ID NO:2).

MHKLAHTSFGTVGMFVNTCIVAKYVIINWEMFSMKKVNNDTVFGTLQLET
LLGDINSIFSEIESEYKMSREEILILLTLWQKGFMTLKEMDRFVEVKPYK
RTRTYNNLVELEWIYKERPVDDERTVIIHFNEKLQQEKVELLNFISDAIA
SPATANQNSLNAIIAV

### Example 6

This example describes the open reading frame (SEQ ID NO:3) of Rlp.

GTGGACTATCAAACTTTCGAAAAGGTCAATAAATTCATAAATGTAAAAGC
GTACATATTTTTTCTCACTCAAGAGTTAAAGCAACAATATAAATTATCAT
TAAAAGAATTGTTGATATTAGCATATTTTTATTACAAAAATGAACACAGT
ATTTCACTAAAAGAAATCATTGGTGACATACTTTACAAACAATCTGATGT
TGTAAAGAACATTAAGTCACTATCTAAAAAAGGATTTATAAATAAGTCTA
GAAACGAAGCAGATGAACGCCGTATTTTTGTTTCAGTTACTCCAATACAA
CGTAAAAAGATTGCTTGTGTTATTAATGAGTTAGATAAAATAATTAAAGG
ATTTAATAAGGAAAGAGACTACATAAAATATCAATGGGCTCCAAAATATA
GCAAAGAATTTTTTATACTTTTTATGAACATTATGTACTCAAAAGATTTT
TTAAATATCGATTTAATTTAACATTTCTTGATTTATCTATCTTATATGTA
ATATACATCTCGAAAAAATGAGATACTAAATTTAAAAGATTTGTTTGAAA
GTATTAGATTTATGTATCCTCAAATTGTTAGGTCAGTTAATAGATTAAAT
AATAAAGGTATGCTAATCAAAGAACGATCCCTTGCAGATGAAAGGATTGT
GTTAATCAAAATAAATAAAATACAATATAACACTATTAAAAGCATATTCA
CACATACTTCCAAGATTCTCAAACCAAGAAAATTTTTCTTTTAAATTTA

### Example 7

This example describes the amino acid sequence of Rlp (SEQ ID NO:4).

MDYQTFEKVNKFINVKAYIFFLTQELKQQYKLSLKELLILAYFYYKNEHS
ISLKEIIGDILYKQSDVVKNIKSLSKKGFINKSRNEADERRIFVSVTPIQ
RKKIACVINELDKITKGFNKERDYIKYQWAPKYSKEFFILFMNIMYSKDF
LKYRFNLTFLDLSILYVISSRKNETLNLKDLFESIRFMYPQIVRSVNRLN
NKGMLIKERSLADERIVLIKINKIQYNTIKSIFTDTSKILKPRKFFF

TABLE 5
A listing of the genes transcriptionally up-regulated by Rot. The effect
of the products of agr on transcription are included for comparison.

| Chip orf no.^{a} | N315 orf no.^{b} | N315 gene^{c} | N315_Description^{d} | agr/sar^{e} | Role Category^{f} |
|---|---|---|---|---|---|
| 4303 | SA2335 | adaB | probable methylated | | |
| | | | DNA-protein | | |
| | | | cysteine | | |
| | | | methyltransferase | | |
| 3994 | SA1736 | aldH | aldehyde | | |
| | | | dehydrogenase | | |
| 2466 | SA2008 | alsS | alpha-acetolactate | | |
| | | | synthase | | |
| 421 | SA2426 | arcD | arginine/oirnithine | agr-up | transport |
| | | | antiporter | | |
| 5372 | | clfB | clumping factor B | | |
| 2462 | SA2336 | clpL | ATP-dependent Clp | sar- | adaptation |
| | | | proteinase chain | down | |
| | | | clpL | | |
| 3833 | | coa | coagulase | | |
| 2163 | SA1253 | ctpA | probable carboxy- | | |
| | | | terminal processing | | |
| | | | proteinase ctpA | | |
| 5110 | SA1164 | dhoM | homoserine | | |
| | | | dehydrogenase | | |
| 1985 | SA0794 | dltB | DltB membrane | | |
| | | | protein | | |
| 1986 | SA0795 | dltC | D-alanyl carrier | | |
| | | | protein | | |
| 4873 | SA0796 | dltD | poly (glycerophosphate | agr- | transport |
| | | | chain) D-alanine | down | |
| | | | transfer protein | | |
| 3193 | SA0436 | dnaX | DNA polymerase III | | |
| | | | gamma and tau | | |
| | | | subunits | | |
| 3383 | | epiA | lantibiotic | | |
| | | | gallidermin | | |
| | | | precursor EpiA | | |
| 84 | SA0602 | fhuA | ferrichrome | | |
| | | | transport ATP- | | |
| | | | binding protein | | |
| 4551 | SA2172 | gltT | proton/sodium- | | |
| | | | glutamate symport | | |
| | | | protein | | |
| 4042 | SA2288 | gtaB | UTP-glucose-1- | | |
| | | | phosphate | | |
| | | | uridyltransferase | | |
| 3087 | SA0376 | guaA | GMP synthase | | |
| | | | (glutamine- | | |
| | | | hydrolyzing) | | |
| 1057 | SA0375 | guaB | inositol- | | |
| | | | monophosphate | | |
| | | | dehydrogenase | | |
| 3534 | SA1412 | hemN | oxygen-independent | | |
| | | | coproporphyrinogen | | |
| | | | oxidase (EC | | |
| | | | 1.3.3.3) III | | |
| 1331 | SA1656 | hit | Hit-like protein | agr- | Miscellaneous |
| | | | involved in cell- | down | |
| | | | cycle regulation | | |
| 1427 | SAS065 | hld | delta-heraolysin | agr-up | Virulence |
| | | | | | factors |
| 1988 | SA0442 | holB | probable DNA | | |
| | | | polymerase III, | | |
| | | | delta prime subunit | | |
| 3984 | SA0189 | hsdR | probable type I | | |
| | | | restriction enzyme | | |
| | | | restriction chain | | |
| 427 | SA2431 | isaB | immunodominant | sar- | Virulence |
| | | | antigen B | down | factors |
| 942 | SA1413 | lepA | GTP-binding protein | | |
| 2883 | SA1505 | lysP | lysine-specific | agr-up | transport |
| | | | permease | | |
| 1565 | SA1458 | lytH | N-acetylmuramoyl-L- | | |
| | | | alanine amidase | | |
| 2234 | SA0251 | lytR | two-component | | |
| | | | response regulator | | |
| 4486 | SA0250 | lytS | two-component | | |
| | | | sensor histidine | | |
| | | | kinase | | |
| 1183 | SA0813 | mnhA | Na+/H+ antiporter | agr- | electron |
| | | | subunit | down | transport |
| 1047 | SA1194 | msrA | peptide methionine | | |
| | | | sulfoxide reductase | | |
| | | | homolog | | |
| 4144 | SA0547 | mvaK1 | mevalonate kinase | | |
| 854 | SA2410 | nrdD | anaerobic | | |
| | | | ribonucleoside- | | |
| | | | triphosphate | | |
| | | | reductase | | |
| 4206 | SA0686 | nrdE | ribonuceloside | | |
| | | | diphosphate | | |
| | | | reductase major | | |
| | | | subunit | | |
| 4205 | SA0685 | nrdI | NrdI protein | | |
| | | | involved in | | |
| | | | ribonucleotide | | |
| | | | reductase function | | |
| 4390 | SA0374 | pbuX | xanthine permease | | |
| 1505 | SA0460 | pth | peptidyl-tRNA | agr- | translation |
| | | | hydrolase | down | elongation |
| 4905 | SA0923 | purM | phosphoribosylform- | sar- | nucleotide |
| | | | ylglycinamidine | down | & nucleic |
| | | | cyclo-ligase PurM | | acid |
| | | | | | metabolism |
| 4137 | SA1718 | putP | high affinity | | |
| | | | proline permease | | |
| 5335 | SA0963 | pycA | pyruvate | | |
| | | | carboxylase | | |
| 2800 | SA0676 | recQ | probable DNA | agr-up | DNA |
| | | | helicase | | replication |
| 1079 | SA1583 | rot | represser of toxins | | |
| | | | Rot | | |
| 3568 | SA0501 | rpoC | RNA polymerase | | |
| | | | beta-prime chain | | |
| 2343 | | sdrC | sdrC protein | sar-up | Virulence |
| | | | | | factors |
| 2125 | SA1869 | sigB | sigma factor B | | |
| 2735 | SA0128 | sodM | superoxide | | |
| | | | dismutase | | |
| 2119 | SA0107 | spa | Immunoglobulin G | agr- | Virulence |
| | | | winding protein A | down | factors |
| | | | precursor | | |
| 4767 | SA1322 | srrB | staphylococcal | | |
| | | | respiratory | | |
| | | | response protein | | |
| | | | SrrB | | |
| 5112 | SA1166 | thrB | homoserine kinase | | |
| | | | homolog | | |
| 3239 | SA1165 | thrC | threonine synthase | | |
| 5075 | SA0373 | xprT | xanthine | | |
| | | | phosphoribosyltrans- | | |
| | | | ferase | | |
| 4253 | SA0003 | | conserved HP | | |
| 3517 | SA0013 | | conserved HP | | |
| 3843 | SA0078 | | HP | | |
| 3698 | SA0173 | | HP, similar to | agr-up | antibiotic |
| | | | surfactin | | production |
| | | | synthetase | | |
| 18 | SA0220 | | HP, similar to | | |
| | | | glycerophospho- | | |
| | | | diester | | |
| | | | phosphodiesterase | | |
| 31 | SA0229 | | HP, similar to | | |
| | | | nickel ABC | | |
| | | | transporter nickel- | | |
| | | | binding protein | | |
| 32 | SA0230 | | conserved HP | | |
| 5437 | SA0246 | | hypotheticl | | |
| | | | protein, similar to | | |
| | | | D-xylulose | | |
| | | | reductase | | |
| 1228 | SA0248 | | HP, similar to | | |
| | | | beta- | | |
| | | | glycosyltransferase | | |
| 2103 | SA0271 | | conserved HP | agr-up | unknown |
| 2077 | SA0291 | | HP | | |
| 2078 | SA0292 | | HP | | |
| 3832 | SA0298 | | HP, similar to | | |
| | | | regulatory protein | | |
| | | | PfoR | | |
| 4153 | SA0310 | | HP | | |
| 2368 | SA0330 | | HP, similar to | | |
| | | | ribosomal-protein- | | |
| | | | serine N- | | |
| | | | acetyltransferase | | |
| 2507 | SA0380 | | conserved HP | | |
| | | | [Pathogenicity | | |
| | | | island SaPIn2] | | |
| 2472 | SA0406 | | HP | | |
| 1563 | SA0407 | | conserved HP | | |
| 1564 | SA0408 | | HP | | |
| 477 | SA0428 | | conserved HP | | |
| 1991 | SA0439 | | HP, similar to | | |
| | | | lysine | | |
| | | | decarboxylase | | |
| 2482 | SA0462 | | HP, similar to low | | |
| | | | temperature | | |
| | | | requirement B | | |
| | | | protein | | |
| 66 | SA0507 | | HP, similar to N- | agr-up | amino acid |
| | | | acyl-L-amino acid | | metabolism |
| | | | amidohydrolase | | |
| 5142 | SA0513 | | conserved HP | | |
| 4157 | SA0517 | | conserved HP | | |
| 600 | SA0523 | | HP, similar to poly | sar-up | cell wall |
| | | | (GP) alpha- | | |
| | | | glucosyltransferase | | |
| | | | (TA biosynthesis) | | |
| 631 | SA0524 | | conserved HP | | |
| 4183 | SA0526 | | conserved HP | | |
| 5101 | SA0541 | | HP, similar to | | |
| | | | cationic amino acid | | |
| | | | transporter | | |
| 5483 | SA0566 | | HP, similar to | | |
| | | | iron-binding | | |
| | | | protein | | |
| 1701 | SA0601 | | conserved HP | | |
| 3305 | SA0613 | | HP | | |
| 1082 | SA0620 | | secretory antigen | | |
| | | | SsaA homologue | | |
| 2907 | SA0622 | | HP, similar to | | |
| | | | AraC/XylS family | | |
| | | | transcriptional | | |
| | | | regulator | | |
| 442 | SA0651 | | HP | | |
| 4090 | SA0657 | | HP, similar to | | |
| | | | hemolysin homologue | | |
| 3259 | SA0675 | | HP, similar to ABC | | |
| | | | transporter ATP- | | |
| | | | binding protein | | |
| 1422 | SA0678 | | HP, similar to | | |
| | | | choline transporter | | |
| 678 | SA0682 | | HP, similar to di- | | |
| | | | tripepride ABC | | |
| | | | transporter | | |
| 392 | SA0739 | | conserved HP | | |
| 1459 | SA0770 | | conserved HP | | |
| 4867 | SA0788 | | conserved HP | | |
| 1187 | SA0817 | | HP, similar to | | |
| | | | NADH-dependent | | |
| | | | flavin oxidoreductase | | |
| 3035 | SA0827 | | HP, similar to ATP- | | |
| | | | dependent nuclease | | |
| | | | subunit B | | |
| 5245 | SA0827 | | HP, similar to ATP- | | |
| | | | dependent nuclease | | |
| | | | subunit B | | |
| 3774 | SA0863 | | conserved HP | | |
| 1754 | SA0867 | | HP, similar to Mg2+ | | |
| | | | transporter | | |
| 1755 | SA0868 | | Na+/H+ antiporter | | |
| | | | homologue | | |
| 1022 | SA0883 | | HP | | |
| 1398 | SA0914 | | reverse complement | agr-up | Miscellaneous |
| | | | of HP, similar to | | |
| | | | chitinase B | | |
| 4669 | SA0968 | | conserved HP | | |
| 4667 | SA0973 | | phosphopantetheine | | |
| | | | adenyltransferase | | |
| | | | homolog | | |
| 4525 | SA1016 | | reverse complement | | |
| | | | of conserved HP | | |
| 4752 | SA1056 | | HP | | |
| 2032 | SA1059 | | methionyl-tRNA | agr-up | aminnoacyl |
| | | | formyltransferase | | tRNA |
| | | | | | synthetases |
| 2679 | SA1121 | | HP, similar to | | |
| | | | processing | | |
| | | | proteinase homolog | | |
| 5471 | SA1131 | | HP, similar to 2- | | |
| | | | oxoacid ferredoxin | | |
| | | | oxidoreductase, | | |
| | | | alpha subunit | | |
| 5175 | SA1132 | | HP, similar to 2- | | |
| | | | oxoacid ferredoxin | | |
| | | | oxidoreductase, | | |
| | | | beta subunit | | |
| 2592 | SA1159 | | HP, similar to two- | | |
| | | | component response | | |
| | | | regulator | | |
| 2971 | SA1199 | | HP, similar to | | |
| | | | anthranilate | | |
| | | | synthase component | | |
| | | | I | | |
| 2454 | SA1320 | | HP | | |
| 457 | SA1613 | | conserved HP | | |
| 329 | SA1625 | | probable | | |
| | | | specificity | | |
| | | | determinant HsdS | | |
| | | | [Pathogenicity | | |
| | | | island SaPIn3] | | |
| 5554 | SA1679 | | HP, similar to D-3- | | |
| | | | phosphoglycerate | | |
| | | | dehydrogenase | | |
| 146 | SA1680 | | conserved HP | | |
| 534 | SA1717 | | glutamyl-tRNAGln | | |
| | | | amidotransferase | | |
| | | | subunit C | | |
| 4713 | SA1877 | | conserved HP | | |
| 3426 | SA1924 | | HP, simialr to | | |
| | | | aldehyde | | |
| | | | dehydrogenase | | |
| 4282 | SA1942 | | conserved HP | | |
| 5232 | SA1943 | | HP | | |
| 168 | SA1986 | | HP | | |
| 2337 | SA2001 | | HP, simialr to | | |
| | | | oxidoreductase, | | |
| | | | aldo/keto reductase | | |
| | | | family | | |
| 1027 | SA2007 | | HP, similar to | | |
| | | | alpha-acetolactate | | |
| | | | decarboxylase | | |
| 322 | SA2019 | | conserved HP | | |
| 5005 | SA2052 | | conserved HP | agr-up | translation |
| 4825 | SA2056 | | HP, similar to | | |
| | | | acriflavin | | |
| | | | resistance protein | | |
| 58 | SA2096 | | conserved HP | | |
| 3907 | SA2097 | | HP, similar to | | |
| | | | secretory antigen | | |
| | | | precursor SsaA | | |
| 2202 | SA2102 | | formate | | |
| | | | dehydrogenase | | |
| | | | homolog | | |
| 5585 | SA2119 | | HP, simialr to | | |
| | | | dehydrogenase | | |
| 2132 | SA2131 | | conserved HP | | |
| 2133 | SA2132 | | HP, simialr to ABC | | |
| | | | transporter (ATP- | | |
| | | | binding protein) | | |
| 616 | SA2133 | | conserved HP | | |
| 846 | SA2156 | | L-lactate permease | | |
| | | | lctP homolog | | |
| 1356 | SA2170 | | HP, similar to | | |
| | | | general stress | | |
| | | | protein 26 | | |
| 1357 | SA2171 | | HP | | |
| 1632 | SA2228 | | HP, similar to | | |
| | | | NA(+)/H(+) | | |
| | | | exchanger | | |
| 1231 | SA2231 | | HP, similar to | | |
| | | | glucose epimerase | | |
| 1232 | SA2232 | | HP, similar to 2- | | |
| | | | dehydropantoate 2- | | |
| | | | reductase | | |
| 1233 | SA2233 | | HP, similar to | | |
| | | | integral membrane | | |
| | | | efflux protein | | |
| 5270 | SA2240 | | HP, similar to | sar- | lipid |
| | | | para-nitrobenzyl | down | metabolism |
| | | | esterase chain A | | |
| 1709 | SA2247 | | conserved HP | | |
| 5065 | SA2256 | | conserved HP | | |
| 748 | SA2261 | | HP, similar to | | |
| | | | efflux pump | | |
| 1917 | SA2265 | | HP | | |
| 1918 | SA2266 | | HP, similar to | | |
| | | | oxidoreductase | | |
| 1175 | SA2284 | | HP, similar to | agr-up | Virulence |
| | | | accumulation- | | factors |
| | | | associated protein | | |
| 1666 | SA2303 | | HP, simialr to | | |
| | | | membrane spanning | | |
| | | | protein | | |
| 3888 | SA2339 | | HP, similar to | | |
| | | | antibiotic | | |
| | | | transport- | | |
| | | | associated protein | | |
| 493 | SA2378 | | conserved HP | agr- | unknown |
| | | | | down | |
| 105 | SA2402 | | acetate-CoA ligase | | |
| | | | (EC 6.2.1.1) | | |
| 855 | SA2409 | | HP, similar to | | |
| | | | anaerobic | | |
| | | | ribonucleotide | | |
| | | | reductase activator | | |
| | | | protein | | |
| 4290 | SA2413 | | sulfite reductase | | |
| | | | (NADPH) (EC | | |
| | | | 1.8.1.2) | | |
| | | | flavoprotein | | |
| 3313 | SA2436 | | HP, similar to | sar-up | bacteriophage |
| | | | phage infection | | related |
| | | | protein | | |
| 2912 | SA2438 | | HP, similar to N- | sar- | coenzyme |
| | | | Carbamoylsarosine | down | metabolism |
| | | | Amidohydrolase | | |
| 2837 | SA2439 | | conserved HP | | |
| 3518 | SA2439 | | conserved HP | | |
| 2232 | SA2440 | | HP | | |
| 4992 | SA2440 | | HP | | |
| 2697 | SA2442 | | preprotein | | |
| | | | translocase secA | | |
| | | | homolog | | |
| 218 | SA2444 | | HP | | |
| 356 | SA2445 | | HP | | |
| 360 | SA2447 | | HP, similar to | agr-up | Virulence |
| | | | streptococcal | | factors |
| | | | hemagglutinin | | |
| | | | protein | | |
| 195 | SA2487 | | HP, similar to rarD | | |
| | | | protein | | |
| 1760 | SA2495 | | HP, similar to HP | | |
| 1759 | SA2496 | | HP | | |
| 1758 | SA2497 | | HP | | |
| 237 | SAS013 | | reverse complement | agr-up | unknown |
| | | | of HP | | |
| | | | [Pathogenicity | | |
| | | | island SaPIn2] | | |
| 873 | SAS088 | | HP | | |
| 17 | | | HP | | |
| 1212 | | | surface protein, | | |
| | | | putative | | |
| 1353 | | | HP | | |
| 1592 | | | HP | | |
| 1597 | | | HP | | |
| 1765 | | | epidermin immunity | agr-up | antibiotic |
| | | | protein F | | production |
| 2076 | | | | | |
| 2255 | | | HP | | |
| 2275 | | | | agr-up | unknown |
| 3391 | | | conserved HP | | |
| 3599 | | | ABC transporter, | | |
| | | | ATP-binding protein | | |
| 4138 | | | conserved HP | | |
| 4550 | | | HP | | |
| 4829 | | | reverse complement | | |
| | | | of HP | | |

^{a}S. aureus GeneChip ORF number.
^{b}ORF number based on the published sequence of strain N315 (Accession no. NC_002745)
^{c,d}Gene name & description based on the published sequence of strain N315 (Accession no. NC_002745)
Note:
For genes not present in N315, the gene name & description are from the COL genome:
^{e,f}Effects of agr and sar & expected metabolic role as described by Dunman et al(Dunman et al., 2001). Up = upregulated; down = downregulated
HP = hypothetical protein; GP = glycerol-phosphate; TA = teichoic acid
TIGR CMR: http://www.tigr.org/tigr-scripts/CMR2/GeneNameSearch.spl?db=gsa

TABLE 6
A listing of the genes transcriptionally down-regulated by Rot. The effect
of the products of agr on transcription are included for comparison.

| Chip orf no.^{a} | N315 orf no.^{b} | M315 gene^{c} | N315_Description^{d} | agr/sar^{e} | Role Category^{f} |
|---|---|---|---|---|---|
| 4303 | SA2335 | adaB | probable methylated | | |
| | | | DNA-protein | | |
| | | | cysteine | | |
| | | | methyltransferase | | |
| 3994 | SA1736 | aldH | aldehyde | | |
| | | | dehydrogenase | | |
| 2466 | SA2008 | alsS | alpha-acetolactate | | |
| | | | synthase | | |
| 421 | SA2426 | arcD | arginine/oirnithine | agr-up | transport |
| | | | antiporter | | |
| 5372 | | clfB | clumping factor B | | |
| 2462 | SA2336 | clpL | ATP-dependent Clp | sar- | adaptation |
| | | | proteinase chain | down | |
| | | | clpL | | |
| 3833 | | coa | coagulase | | |
| 2163 | SA1253 | ctpA | probable carboxy- | | |
| | | | terminal processing | | |
| | | | proteinase ctpA | | |
| 5110 | SA1164 | dhoM | homoserine | | |
| | | | dehydrogenase | | |
| 1985 | SA0794 | dltB | DltB membrane | | |
| | | | protein | | |
| 1986 | SA0795 | dltC | D-alanyl carrier | | |
| | | | protein | | |
| 4873 | SA0796 | dltD | poly(glycerophosphate | agr- | transport |
| | | | chain) D-alanine | down | |
| | | | transfer protein | | |
| 3193 | SA0436 | dnaX | DNA polymerase III | | |
| | | | gamma and tau | | |
| | | | subunits | | |
| 3383 | | epiA | lantibiotic | | |
| | | | gallidermin | | |
| | | | precursor EpiA | | |
| 84 | SA0602 | fhuA | ferrichrome | | |
| | | | transport ATP- | | |
| | | | binding protein | | |
| 4551 | SA2172 | gltT | proton/sodium- | | |
| | | | glutamate symport | | |
| | | | protein | | |
| 4042 | SA2288 | gtaB | UTP-glucose-1- | | |
| | | | phosphate | | |
| | | | uridyltransferase | | |
| 3087 | SA0376 | guaA | GMP synthase | | |
| | | | (glutamine- | | |
| | | | hydrolyzing) | | |
| 1057 | SA0375 | guaB | inositol- | | |
| | | | monophosphate | | |
| | | | dehydrogenase | | |
| 3534 | SA1412 | hemN | oxygen-independent | | |
| | | | coproporphyrinogen | | |
| | | | oxidase (EC | | |
| | | | 1.3.3.3) III | | |
| 1331 | SA1656 | hit | Hit-like protein | agr- | Miscellaneous |
| | | | involved in cell- | down | |
| | | | cycle regulation | | |
| 1427 | SAS065 | hld | delta-hemolysin | agr-up | Virulence |
| | | | | | factors |
| 1988 | SA0442 | holB | probable DNA | | |
| | | | polymerase III, | | |
| | | | delta prime subunit | | |
| 3984 | SA0189 | hsdR | probable type I | | |
| | | | restriction enzyme | | |
| | | | restriction chain | | |
| 427 | SA2431 | isaB | immunodominant | sar- | Virulence |
| | | | antigen B | down | factors |
| 942 | SA1413 | lepA | GTP-binding protein | | |
| 2883 | SA1505 | lysP | lysine-specific | agr-up | transport |
| | | | permease | | |
| 1565 | SA1458 | lytH | N-acetylmuramoyl-L- | | |
| | | | alanine amidase | | |
| 2234 | SA0251 | lytR | two-component | | |
| | | | response regulator | | |
| 4486 | SA0250 | lytS | two-component | | |
| | | | sensor histidine | | |
| | | | kinase | | |
| 1183 | SA0813 | mnhA | Na+/H+ antiporter | agr- | electron |
| | | | subunit | down | transport |
| 1047 | SA1194 | msrA | peptide methionine | | |
| | | | sulfoxide reductase | | |
| | | | homolog | | |
| 4144 | SA0547 | mvaK1 | mevalonate kinase | | |
| 854 | SA2410 | nrdD | anaerobic | | |
| | | | ribonucleoside- | | |
| | | | triphosphate | | |
| | | | reductase | | |
| 4206 | SA0686 | nrdE | ribonuceloside | | |
| | | | diphosphate | | |
| | | | reductase major | | |
| | | | subunit | | |
| 4205 | SA0685 | nrdI | NrdI protein | | |
| | | | involved in | | |
| | | | ribonucleotide | | |
| | | | reductase function | | |
| 4390 | SA0374 | pbuX | xanthine permease | | |
| 1505 | SA0460 | pth | peptidyl-tRNA | agr- | translation |
| | | | hydrolase | down | elongation |
| 4905 | SA0923 | purM | phosphoribosylformyl- | sar- | nucleotide |
| | | | glycinamidine | down | & nucleic |
| | | | cyclo-ligase PurM | | acid |
| | | | | | metabolism |
| 4137 | SA1718 | putP | high affinity | | |
| | | | proline permease | | |
| 5335 | SA0963 | pycA | pyruvate | | |
| | | | carboxylase | | |
| 2800 | SA0676 | recQ | probable DNA | agr-up | DNA |
| | | | helicase | | replication |
| 1079 | SA1583 | rot | represser of toxins | | |
| | | | Rot | | |
| 3568 | SA0501 | rpoC | RNA polymerase | | |
| | | | oeta-prime chain | | |
| 2343 | | sdrC | sdrC protein | sar-up | Virulence |
| | | | | | factors |
| 2125 | SA1869 | sigB | sigma factor B | | |
| 2735 | SA0128 | sodM | superoxide | | |
| | | | dismutase | | |
| 2119 | SA0107 | spa | Immunoglobulin G | agr- | Virulence |
| | | | binding protein A | down | factors |
| | | | precursor | | |
| 4767 | SA1322 | srrB | staphylococcal | | |
| | | | respiratory | | |
| | | | response protein | | |
| | | | SrrB | | |
| 5112 | SA1166 | thrB | homoserine kinase | | |
| | | | homolog | | |
| 3239 | SA1165 | thrC | threonine synthase | | |
| 5075 | SA0373 | xprT | xanthine | | |
| | | | phosphoribosyltrans- | | |
| | | | ferase | | |
| 4253 | SA0003 | | conserved HP | | |
| 3517 | SA0013 | | conserved HP | | |
| 3843 | SA0078 | | HP | | |
| 3698 | SA0173 | | HP, similar to | agr-up | antibiotic |
| | | | surfactin | | production |
| | | | synthetase | | |
| 18 | SA0220 | | HP, similar to | | |
| | | | glycerophospho- | | |
| | | | diester | | |
| | | | phosphodiesterase | | |
| 31 | SA0229 | | HP, similar to | | |
| | | | nickel ABC | | |
| | | | transporter nickel- | | |
| | | | binding protein | | |
| 32 | SA0230 | | conserved HP | | |
| 5437 | SA0246 | | hypotheticl | | |
| | | | protein, similar to | | |
| | | | D-xylulose | | |
| | | | reductase | | |
| 1228 | SA0248 | | HP, similar to | | |
| | | | beta- | | |
| | | | glycosyltransferase | | |
| 2103 | SA0271 | | conserved HP | agr-up | unknown |
| 2077 | SA0291 | | HP | | |
| 2078 | SA0292 | | HP | | |
| 3832 | SA0298 | | HP, similar to | | |
| | | | regulatory protein | | |
| | | | PfoR | | |
| 4153 | SA0310 | | HP | | |
| 2368 | SA0330 | | HP, similar to | | |
| | | | ribosomal-protein- | | |
| | | | serine N- | | |
| | | | acetyltransferase | | |
| 2507 | SA0380 | | conserved HP | | |
| | | | [Pathogenicity | | |
| | | | island SaPIn2] | | |
| 2472 | SA0406 | | HP | | |
| 1563 | SA0407 | | conserved HP | | |
| 1564 | SA0408 | | HP | | |
| 477 | SA0428 | | conserved HP | | |
| 1991 | SA0439 | | HP, similar to | | |
| | | | lysine | | |
| | | | decarboxylase | | |
| 2482 | SA0462 | | HP, similar to low | | |
| | | | temperature | | |
| | | | requirement B | | |
| | | | protein | | |
| 66 | SA0507 | | HP, similar to N- | agr-up | amino acid |
| | | | acyl-L-amino acid | | metabolism |
| | | | amidohydrolase | | |
| 5142 | SA0513 | | conserved HP | | |
| 4157 | SA0517 | | conserved HP | | |
| 600 | SA0523 | | HP, similar to poly | sar-up | cell wall |
| | | | (GP) alpha- | | |
| | | | glucosyltransferase | | |
| | | | (TA biosynthesis) | | |
| 631 | SA0524 | | conserved HP | | |
| 4183 | SA0526 | | conserved HP | | |
| 5101 | SA0541 | | HP, similar to | | |
| | | | cationic amino acid | | |
| | | | transporter | | |
| 5483 | SA0566 | | HP, similar to | | |
| | | | iron-binding | | |
| | | | protein | | |
| 1701 | SA0601 | | conserved HP | | |
| 3305 | SA0613 | | HP | | |
| 1082 | SA0620 | | secretory antigen | | |
| | | | SsaA homologue | | |
| 2907 | SA0622 | | HP, similar to | | |
| | | | AraC/XylS family | | |
| | | | transcriptional | | |
| | | | regulator | | |
| 442 | SA0651 | | HP | | |
| 4090 | SA0657 | | HP, similar to | | |
| | | | hemolysin homologue | | |
| 3259 | SA0675 | | HP, similar to ABC | | |
| | | | transporter ATP- | | |
| | | | binding protein | | |
| 1422 | SA0678 | | HP, similar to | | |
| | | | choline transporter | | |
| 678 | SA0682 | | HP, similar to di- | | |
| | | | tripepride ABC | | |
| | | | transporter | | |
| 392 | SA0739 | | conserved HP | | |
| 1459 | SA0770 | | conserved HP | | |
| 4867 | SA0788 | | conserved HP | | |
| 1187 | SA0817 | | HP, similar to | | |
| | | | NADH-dependent | | |
| | | | flavin | | |
| | | | oxidoreductase | | |
| 3035 | SA0827 | | HP, similar to ATP- | | |
| | | | dependent nuclease | | |
| | | | subunit B | | |
| 5245 | SA0827 | | HP, similar to ATP- | | |
| | | | dependent nuclease | | |
| | | | subunit B | | |
| 3774 | SA0863 | | conserved HP | | |
| 1754 | SA0867 | | HP, similar to Mg2+ | | |
| | | | transporter | | |
| 1755 | SA0868 | | Na+/H+ antiporter | | |
| | | | homologue | | |
| 1022 | SA0883 | | HP | | |
| 1398 | SA0914 | | reverse complement | agr-up | Miscellaneous |
| | | | of HP, similar to | | |
| | | | chitinase B | | |
| 4669 | SA0968 | | conserved HP | | |
| 4667 | SA0973 | | phosphopantetheine | | |
| | | | adenyltransferase | | |
| | | | homolog | | |
| 4525 | SA1016 | | reverse complement | | |
| | | | of conserved HP | | |
| 4752 | SA1056 | | HP | | |
| 2032 | SA1059 | | methionyl-tRNA | agr-up | aminnoacyl |
| | | | formyltransferase | | tRNA |
| | | | | | synthetases |
| 2679 | SA1121 | | HP, similar to | | |
| | | | processing | | |
| | | | proteinase homolog | | |
| 5471 | SA1131 | | HP, similar to 2- | | |
| | | | oxoacid ferredoxin | | |
| | | | oxidoreductase, | | |
| | | | alpha subunit | | |
| 5175 | SA1132 | | HP, similar to 2- | | |
| | | | oxoacid ferredoxin | | |
| | | | oxidoreductase, | | |
| | | | beta subunit | | |
| 2592 | SA1159 | | HP, similar to two- | | |
| | | | component response | | |
| | | | regulator | | |
| 2971 | SA1199 | | HP, similar to | | |
| | | | anthranilate | | |
| | | | synthase component | | |
| | | | I | | |
| 2454 | SA1320 | | HP | | |
| 457 | SA1613 | | conserved HP | | |
| 329 | SA1625 | | probable | | |
| | | | specificity | | |
| | | | determinant HsdS | | |
| | | | [Pathogenicity | | |
| | | | island SaPIn3] | | |
| 5554 | SA1679 | | HP, similar to D-3- | | |
| | | | phosphoglycerate | | |
| | | | dehydrogenase | | |
| 146 | SA1680 | | conserved HP | | |
| 534 | SA1717 | | glutamyl-tRNAGln | | |
| | | | amidotransferase | | |
| | | | subunit C | | |
| 4713 | SA1877 | | conserved HP | | |
| 3426 | SA1924 | | HP, simialr to | | |
| | | | aldehyde | | |
| | | | dehydrogenase | | |
| 4282 | SA1942 | | conserved HP | | |
| 5232 | SA1943 | | HP | | |
| 168 | SA1986 | | HP | | |
| 2337 | SA2001 | | HP, simialr to | | |
| | | | oxidoreductase, | | |
| | | | aldo/keto reductase | | |
| | | | family | | |
| 1027 | SA2007 | | HP, similar to | | |
| | | | alpha-acetolactate | | |
| | | | decarboxylase | | |
| 322 | SA2019 | | conserved HP | | |
| 5005 | SA2052 | | conserved HP | agr-up | translation |
| 4825 | SA2056 | | HP, similar to | | |
| | | | acriflavin | | |
| | | | resistance protein | | |
| 58 | SA2096 | | conserved HP | | |
| 3907 | SA2097 | | HP, similar to | | |
| | | | secretory antigen | | |
| | | | precursor SsaA | | |
| 2202 | SA2102 | | formate | | |
| | | | dehydrogenase | | |
| | | | homolog | | |
| 5585 | SA2119 | | HP, simialr to | | |
| | | | dehydrogenase | | |
| 2132 | SA2131 | | conserved HP | | |
| 2133 | SA2132 | | HP, simialr to ABC | | |
| | | | transporter (ATP- | | |
| | | | binding protein) | | |
| 616 | SA2133 | | conserved HP | | |
| 846 | SA2156 | | L-lactate permease | | |
| | | | lctP homolog | | |
| 1356 | SA2170 | | HP, similar to | | |
| | | | general stress | | |
| | | | protein 26 | | |
| 1357 | SA2171 | | HP | | |
| 1632 | SA2228 | | HP, similar to | | |
| | | | NA(+)/H(+) | | |
| | | | exchanger | | |
| 1231 | SA2231 | | HP, similar to | | |
| | | | glucose epimerase | | |
| 1232 | SA2232 | | HP, similar to 2- | | |
| | | | dehydropantoate 2- | | |
| | | | reductase | | |
| 1233 | SA2233 | | HP, similar to | | |
| | | | integral membrane | | |
| | | | efflux protein | | |
| 5270 | SA2240 | | HP, similar to | sar- | lipid |
| | | | para-nitrobenzyl | down | metabolism |
| | | | esterase chain A | | |
| 1709 | SA2247 | | conserved HP | | |
| 5065 | SA2256 | | conserved HP | | |
| 748 | SA2261 | | HP, similar to | | |
| | | | efflux pump | | |
| 1917 | SA2265 | | HP | | |
| 1918 | SA2266 | | HP, similar to | | |
| | | | oxidoreductase | | |
| 1175 | SA2284 | | HP, similar to | agr-up | Virulence |
| | | | accumulation- | | factors |
| | | | associated protein | | |
| 1666 | SA2303 | | HP, simialr to | | |
| | | | membrane spanning | | |
| | | | protein | | |
| 3888 | SA2339 | | HP, similar to | | |
| | | | antibiotic | | |
| | | | transport- | | |
| | | | associated protein | | |
| 493 | SA2378 | | conserved HP | agr- | unknown |
| | | | | down | |
| 105 | SA2402 | | acetate-CoA ligase | | |
| | | | (EC 6.2.1.1) | | |
| 855 | SA2409 | | HP, similar to | | |
| | | | anaerobic | | |
| | | | ribonucleotide | | |
| | | | reductase activator | | |
| | | | protein | | |
| 4290 | SA2413 | | sulfite reductase | | |
| | | | (NADPH) (EC | | |
| | | | 1.8.1.2) | | |
| | | | flavoprotein | | |
| 3313 | SA2436 | | HP, similar to | sar-up | bacteriophage |
| | | | phage infection | | related |
| | | | protein | | |
| 2912 | SA2438 | | HP, similar to N- | sar- | coenzyme |
| | | | Carbamoylsarcosine | down | metabolism |
| | | | Amidohydrolase | | |
| 2837 | SA2439 | | conserved HP | | |
| 3518 | SA2439 | | conserved HP | | |
| 2232 | SA2440 | | HP | | |
| 4992 | SA2440 | | HP | | |
| 2697 | SA2442 | | preprotein | | |
| | | | translocase secA | | |
| | | | homolog | | |
| 218 | SA2444 | | HP | | |
| 356 | SA2445 | | HP | | |
| 360 | SA2447 | | HP, similar to | agr-up | Virulence |
| | | | streptococcal | | factors |
| | | | hemagglutinin | | |
| | | | protein | | |
| 195 | SA2487 | | HP, similar to rarD | | |
| | | | protein | | |
| 1760 | SA2495 | | HP, similar to HP | | |
| 1759 | SA2496 | | HP | | |
| 1758 | SA2497 | | HP | | |
| 237 | SAS013 | | reverse complement | agr-up | unknown |
| | | | of HP | | |
| | | | [Pathogenicity | | |
| | | | island SaPIn2] | | |
| 873 | SAS088 | | HP | | |
| 17 | | | HP | | |
| 1212 | | | surface protein, | | |
| | | | putative | | |
| 1353 | | | HP | | |
| 1592 | | | HP | | |
| 1597 | | | HP | | |
| 1765 | | | epidermin immunity | agr-up | antibiotic |
| | | | protein F | | production |
| 2076 | | | | | |
| 2255 | | | HP | | |
| 2275 | | | | agr-up | unknown |
| 3391 | | | conserved HP | | |
| 3599 | | | ABC transporter, | | |
| | | | ATP-binding protein | | |
| 4138 | | | conserved HP | | |
| 4550 | | | HP | | |
| 4829 | | | reverse complement | | |
| | | | of HP | | |

^{a}S. aureus GeneChip ORF number.
^{b}ORF number based on the published sequence of strain N315 (Accession no. NC_002745)
^{c,d}Gene name & description based on the published sequence of strain N315 (Accession no. NC_002745)
Note:
For genes not present in N315, the gene name & description are from the COL genome:
^{e,f}Effects of agr and sar & expected metabolic role as described by Dunman et al(Dunman et al., 2001). Up = upregulated; down = downregulated
HP = hypothetical protein; GP = glycerol-phosphate; TA = teichoic acid
TIGR CMR: http://www.tigr.org/tigr-scripts/CMR2/GeneNameSearch.spl?db=gsa

### REFERENCES CITED

Abbas-Ali, B. and G. Colman. The characteristics of extracellular protein secretion by Staphylococcus aureus (Wood 46) and their relationship to the regulation of α-toxin formation. J. Gen. Microbiol. 99:277-282, 1977.

Abdelnour, A., Arvidson, S., Bremell, T., Ryden, C. and A. Tarkowski. The accessory gene regulator (agr) controls Staphylococcus aureus virulence in a murine model. Infect. Immun. 61:3879-3895, 1993.

Altuvia, S. and E. G. Wagner. Switching on and off with RNA. Proc. Natl. Acad. Sci. U.S.A. 97:9824-9826, 2000.

Ausubel, F. A., Brent, R., Kingston, R. E., Moore, D. D., Steidman, J. G., Smith, J. A., and K. Struhl (ed.) Current Protocols in Molecular Biology. Greene Publishing and Wiley-Interscience, New York, 1996.

Balaban, N. and R. P. Novick. Translation of RNA, the Staphylococcus aureus agr regulatory RNA molecule, can be activated by a 3′-end deletion. FEMS Microbiol. Lett. 133: 155-161, 1995.

Bayer, M. G., Heinrichs, J. H. and A. L. Cheung. The molecular architecture of the sar locus in Staphylococcus aureus. J. Bacteriol. 178:4563-4570, 1996.

Bergdoll, M. S. Regulation and control of toxic shock syndrome toxin 1: overview. Rev. Infect. Dis. 1:S142-122, 1989.

Börklind, A. and S. Arvidson. Mutants of Staphylococcus aureus affected in the regulation of exoprotein synthesis. FEMS Microbiol. Lett. 7:203-206, 1980.

Campbell, In: Monoclonal Antibody Technology, Laboratory Techniques in Biochemistry and Molecular Biology, Vol. 13, Burden and Von Knippenberg, Eds. P. 75-83, Amsterdam, Elsview, 1984.

Chan, P. F. and S. J. Foster. Role of SarA in virulence determinant production and environmental signal transduction in Staphylococcus aureus. J. Bacteriol. 180: 6232-6241, 1998.

Cheung, A. L., Eberhardt, K. J., Chung, E., Yeaman, M. R., Sullam, P. M., Ramos, M. and A. S. Bayer. Diminished virulence of a sar-/agr-mutant of Staphylococcus aureus in the rabbit model of endocarditis. J. Clin. Invest. 94:1815-1822, 1994.

Cheung, A. L., Schmidt, K., Bateman, B. and A. C. Manna. SarS, a SarA homolog repressible by agr, is an activator of protein A synthesis in Staphylococcus aureus. Infect Immun 69:2448-2455, 2001.

Cheung, A. L. and S. J. Projan. Cloning and sequencing of sarA of Staphylococcus aureus, a gene required for the expression of agr. J. Bacteriol. 176:4168-4172, 1994.

Cheung, A. L., Woltz, C., Yeaman, M. R., and A. S. Bayer. Insertional inactivation of a chromosomal locus that modulates expression of potential virulence determinants in Staphylococcus aureus. J. Bacteriol. 177:3220-3226, 1995.

Cheung, A. L., Eberhardt K. and J. H. Heinrichs. Regulation of protein A synthesis by the sar and agr loci of Staphylococcus aureus. Infect. Immun. 65:2243-2249, 1997.

Cheung, A. L. and S. J. Projan. Cloning and sequencing of sarA of Staphylococcus aureus, a gene required for the expression of agr. J. Bacteriol. 176:4168-4172, 1994.

Cheung, A. L., Woltz, C., Yearman, M. R. and A. S. Bayer. Insertional inactivation of a chromosomal locus that modulates expression of potential virulence determinants in Staphylococcus aureus. J. Bacteriol. 177:3220-3226, 1995.

Chien, Y., Manna, A. C. and A. L. Cheung. SarA level is a determinant of agr activation in Staphylococcus aureus. Molec. Microbiol. 39:991-1001, 1998.

Chien, Y., Manna, A. C., Porjan, S. J. and A. L. Cheung. SarA, a global regulator of virulence determinants in Staphylococcus aureus, binds to a conserved motif essential for sar-dependent gene regulation. J. Biol. Chem. 24: 37169-37176, 1999.

Chou, P. Y. and G. D. Fasman. Conformational Parameters for Amino Acids in Helical, β-Sheet, and Random Coil Regions Calculated from Proteins. Biochemistry 13:211-222, 1974.

Chou, P. Y. and G. D. Fasman. Prediction of the Secondary Structure of Proteins from Their Amino Acid Sequence. Adv. Enzymol. Relat. Areas Mol. Biol. 47:45-148, 1978a.

Chou, P. Y. and G. D. Fasman. Empirical Predictions of Protein Conformation. Ann. Rev. Biochem. 47:251-276, 1978b.

Chou, P. Y. and G. D. Fasman. Prediction of β-Turns. Biophys. J., 26:367-384, 1979.

Coen, D. Enzymatic amplification of DNA by PCR: Standard procedures and optimization, p. 15.1.1-15.1.7. In Ausubel, F. A., Brent R., Kingston, R. E., Moore D. D., Seidman, J. G., Smith, J. A. and K. Struhl, (ed.), Current Protocols in Molecular Biology. Greene Publishing and Wiley-Interscience, New York, 1992.

Darouiche, R. O., Landon, G. C., Patti, J. M., Nguyen, L. L., Fernau, R. C., McDevitt, D., Greene, C., Foster, T. and M. Klima. Role of Staphylococcus aureus surface adhesins in orthopaedic device infections: are results model-dependent? J. Med. Microbiol. 46:75-79, 1997.

Dassy, B., Hogan, T., Foster, T. J. and M. Fournier. Involvement of the accessory gene regulator (agr) in expression of type 5 capsular polysaccharide by Staphylococcus aureus. J. Gen. Microbiol. 139:1301-1306, 1993.

Deora, R., Tseng, T. and T. K. Misra. Alternative transcription factor sigmaSB of Staphylococcus aureus: characterization and role in transcription of the global regulatory locus sar. J. Bacteriol. 179:6355-6359, 1997.

Elek, S. and E. Levy. Distribution of haemolysin in pathogenic and non-pathogenic staphylococci. J. Path. Bacteriol. 62:541-554, 1950.

Fournier, B., Klier, A. and G. Rapoport. The two-component system ArlS—ArlR is a regulator of virulence gene expression in Staphylococcus aureus. Mol. Microbiol. 41:247-261, 2001.

Fujimoto, D. F. and K. W. Bales. Opposing roles of the Staphylococcus aureus virulence regulators, Agr and Sar, in Triton X-100 and penicillin-induced autolysis. J. Bacteriol. 180:3724-3726, 1998.

Gefter, M. L., Margulies, D. H. and M. D. Scharff. A simple method for polyethylene glycol-promoted hybridization of mouse myeloma cells. Somatic Cell Genet., 3:231-236, 1977.

Gillaspy, A. F., Hickmon, S. G., Skinner, R. A., Thomas, J. R., Nelson, C. L. and M. S. Smeltzer. 1995. Role of the accessory gene regulator (agr) in pathogenesis of staphylococcal osteomyelitis. Infect. Immun. 63:3373-3380, 1995.

Giraudo, A. T., Cheung, A. L. and R. Nagel. The sae locus of Staphylococcus aureus controls exoprotein synthesis at the transcriptional level. Arch. Microbiol. 168:53-58, 1997.

Giraudo, A. T., Raspanti, C. G., Calzolari, A. and R. Nagel. Characterization of a Tn551-mutant of Staphylococcus aureus defective in the production of several exoproteins. Can. J. Microbiol. 40:677-681, 1994.

Goding, “Monoclonal Antibodies: Principles and Practice,” 2nd Edition, Academic Press, Orlando, Fla., pp. 60-74, 1986.

Goerke, C., Campana, S., Bayer, M. G., Doring, G., Botzenhart, K and C. Wolz. Direct quantitative transcript analysis of the agr regulon of Staphylococcus aureus during human infection in comparison to the expression profile in vitro. Infect. Immun. 68:1304-1311, 2000.

Gopalan, V., Vioque, A. and S. Altman. RNase P: Varieties and uses. J. Biol. Chem. 277:6759-62, 2001.

Harlow, E. and D. Lane (ed.) Antibodies: A Laboratory Manual, Cold Springs Harbor Laboratory, N.Y., 1988.

Hart, M. E., Smeltzer, M. S. and J. J. Iandolo, Phenotypic characterization of xpr, a global regulator of extra-cellular virulence factors in Staphylococcus aureus. Infect Immun 61:919-925, 1993

Heinrichs, J., Bayer, M. and A. Cheung. Characterization of the sar locus and its interaction with agr in Staphylococcus aureus. J. Bacteriol. 178:418-423, 1996.

Inouye, S. and M. Inouye. Up-promoter mutations in the lpp gene of Escherichia coli. Nucleic Acids Res. 13:3101-3110, 1985.

Janzon, L. and S. Arvidson. The role of the delta-lysin gene (hld) in the regulation of virulence genes by the accessory gene regulator (agr) in Staphylococcus aureus. EMBO J. 9:1391-1319, 1990.

Ji., G., Beavis, R. C. and R. P. Novick. Cell density control of staphylococcal virulence mediated by an octapeptide pheromone. Proc. Natl. Acad. Sci. (U.S.A.) 92: 12055-12059, 1995.

Ji, G., Beavis, R. C. and R. P. Novick. Bacterial interference caused by autoinducing peptide variants. Science. 276:2027-2030, 1997.

Karzai, A. W., Susskind, M. M. and R. T. Sauer. SmpB, a unique RNA-binding protein essential for the peptide-tagging activity of SsrA (tmRNA). EMBO J.18:3793-3799, 1999.

Kernodle, D. S., McGraw, P. A., Barg, N. L., Menzies, B. E., Voladri, R. K. and S. Harshmanm. Growth of Staphylococcus aureus with nafcillin in vitro induces alpha-toxin production and increases the lethal activity of sterile broth filtrates in a murine model. J. Infect. Dis. 172:410-419, 1995.

Kornblum, J., Kreiswirth, N., Projan, S. J., Ross, H. and R. P. Novick. agr: a polycistronic locus regulating exoprotein synthesis in Staphylococcus aureus. p. 373-402. In R. P. Novick (ed.), Molecular Biology of the staphylococci. VCTL Publishers, New York, 1990.

Kraemer, G. R. and J. J. Iandolo, High-frequency transformation of Staphylococcus aureus by electroporation. Current Microbiol. 21:373-376, 1990.

Kullik, I., Giachinom P. and T. Fuchs. Deletion of the alternative sigma factor sigmaB in Staphylococcus aureus reveals its function as a global regulator of virulence genes. J. Bacteriol. 180:4814-4820, 1998.

Kuroda, M., Ohta, T., Uchiyama, I., Baba, T., Yuzawa, H., Kobayashi, I., Cui, L., Oguchi, A., Aoki, K., Nagai, Y., Lian, J., Ito, T., Kanamori, M., Matsumaru, H., Maruyama, A., Murakami, H., Hosoyama, A., Mizutani-Ui, Y., Takahashi, N. K, Sawano, T., Inoue, R., Kaito, C., Sekimizu, K., Hirakawa, H., Kuhara, S., Goto, S., Yabuzaki, J., Kanehisa, M., Yamashita, A., Oshima, K., Furuya, K., Yoshino, C., Shiba, T., Hattori, M., Ogasawara, N., Hayashi, H. and K. Hiramatsu. Whole genome sequencing of meticillin-resistant Staphylococcus aureus. Lancet. 357:1225-1240, 2001.

Kyte, J. and R. F. Doolittle. A simple method for displaying the hydropathic character of a protein. J. Mol. Biol. 157: 105-132, 1982.

Lease, R. A., Cusick, M. E. and M. Belfort. Riboregulation in Escherichia coli: DsrA RNA acts by RNA:RNA interactions at multiple loci. Proc. Natl. Acad. Sci. U.S.A. 95:12456-12461, 1998.

Lina, G., Jarraurd, S., Ji, G., Greenland, T., Pedraza, A., Etienne, J., Novick, R. P. and F. Vandenesch. Transmembrane topology and histidine kinase activity of AgrC, the agr signal receptor in Staphylococcus aureus. Mol. Microbiol 28:655-662, 1998.

Liu, Y., Manna, A., Li, R., Martin, W. E., Murphy, R. C., Cheung, A. L. and G. Zhang. Crystal structure of the SarR protein from Staphylococcus aureus. Proc. Natl. Acad. Sci. U.S.A. 98:6877-6882, 2001.

Ludwig, A., Tengel, C., Bauer, S., Bubert, A., Benz, R., Mollenkopf, H. J. and W. Goebel. SlyA, a regulatory protein from Salmonella typhimurium, induces a haemolytic and pore-forming protein in Escherichia coli. Mol. Gen. Genet. 249:474-486, 1995.

Manna, A. and A. L. Cheung. Characterization of sarR, a modulator of sar expression in Staphylococcus aureus. Infect. Immun. 69:885-889, 2001.

McNamara, P. J. and J. J. Iadnolo. Genetic instability of the global regulator agr explains the phenotype of the xpr mutation in Staphylococcus aureus KSI905.1. J. Bacteriol. 180:2609-2615, 1998.

McNamara, P. J., Milligan-Monroe, K. and Proctor, R. A. Identification, cloning, and initial characterization of rot, a locus encoding a repressor of extracellular toxins in Staphylococcus aureus. J. Bacteriol. 182:3197-3203, 2000.

Mills, J. T., Dodel, A. W. and E. H. Kass. Regulation of staphylococcal toxic shock syndrome toxin-1 and total exoprotein production by magnesium ion, Infect. Immun. 53:663-670, 1986.

Morfeldt, E. Tegmark, K. and S. Arvidson. Transcriptional control of the agr-dependent virulence gene regulator, RNAIII, in Staphylococcus aureus. Mol. Microbiol. 21:1227-1237, 1996.

Morfeldt, E., Taylor, D., von Gabain, A. and S. Arvidson. Activation of alpha-toxin translation in Staphylococcus aureus by the trans-encoded antisense RNA, RNAIII. EMBO J. 14:4569-4577, 1995.

Nakamura et al., Enzyme Immunoassays: Heterogeneous and Homogeneous Systems, Chapter 27.

Novick, R. Signal transduction in staphylococci and other gram-positive cocci, p. 39-53. In Rappuoli, R., Scarlato, V., and Arico, B., (ed.), Signal transduction and bacterial virulence. R. G. Landes Company, Austin, 1995.

Novick, R. P., Projan, S. J., Kornblum, J., Ross, H. F., Ji, G., Kreiswirth, B., Bandenesch, F. and S. Moghazeh. The agrP2 operon: an autocatalytic sensory transduction system in Staphylococcus aureus. Mol. Gen. Genet. 248:446-458, 1995.

Novick, R. P., Kornblum, J., Kreisworth, B., Projan, S. and H. Ross. Regulation of post exponential phase exoprotein synthesis in Staphylococcus aureus, p. 9-18. In Ayout, E. M., Cassell, G. H., Branche, W. C., and T. J. Henery (ed.), Microbial Determinants of virulence and hot responses. American Society for Microbiology, Washington, D.C., 1990.

Novick, R. P., Ross, H. F., Projan, S. J., Kornblum, J., Kreiswirth, B. and S. Moghazeh. Synthesis of staphylococcal virulence factors is controlled by a regulatory RNA molecule. EMBO J. 12:3967-3975, 1993.

Palma, M. and A. L. Cheung. Sigma(B) activity in Staphylococcus aureus is controlled by RsbU and an additional factor(s) during bacterial growth. Infect. Immun. 69:7858-65, 2001.

Peng, H. -L., Novick, R. P., Kreiswirth, B., Kornblum, J. and P. Schlievert. Cloning, characterization, and sequence of an accessory gene regulator (agr) in Staphylococcus aureus J. Bacteriol. 170:4365-4372, 1988.

Rampone, H., Martinez, G. L., Giraudo, A. T., Calzolari, A. and R. Nagel. In vivo expression of exoprotein synthesis with a Sae mutant of Staphylococcus aureus. Can. J. Vet. Res. 60:237-240, 1996.

Rechtin T. M., Gillaspy A. F., Schumacher M. A., Brennan R. G., Smeltzer M. S. and B. K. Hurlburt. Characterization of the SarA virulence gene regulator of Staphylococcus aureus. Molec. Mcrobiol. 33:307-316, 1999.

Regassa, L. B. and M. J. Bentley. Alkaline pH decreases expression of the accessory gene regulator (agr) in Staphylococcus aureus. J. Bacteriol. 174:5095-5100, 1992

Regassa, L. B., Novick, R. P. and M. J. Bentley. Glucose and nonmaintained pH decrease expression of the accessory gene regulator (agr) in Staphylococcus aureus. Infect. Immun. 60:3381-3388, 1992.

Romeo, T. Global regulation by the small RNA-binding protein CsrA and the non-coding RNA molecule CsrB. Mol. Microbiol. 29:1321-1330, 1998.

Ruppen, M. E., Van Alstine, G. L. and L. Band. Control of intracellular serine protease expression in Bacillus subtilis. J. Bacteriol. 170:136-140, 1988.

Sambrook, J., Russell, D. W, and J. Sambrook. Molecular cloning: A Laboratory Manual, 2nd ed. Cold Spring Harbor Laboratory Press, Cold Spring Harbor, N.Y., 1989.

Schmidt, K. A., Manna, A. C., Gill, S. and A. L. Cheung. SarT, a repressor of alpha-hemolysin in Staphylococcus aureus. Infect. Immun. 69:4749-4758, 2001.

Schumacher, M. A., Hurlburt, B. K. and R. G. Brennan. Crystal structures of SarA, a pleiotropic regulator of virulence genes in S. aureus. Nature 409:215-219, 2001a.

Schumacher, M. A., Hurlburt, B. K. and R. G. Brennan. Correction: Crystal structures of SarA, a pleiotropic regulator of virulence genes in S. aureus. Nature 409:215-219, 2001b.

Sledjeski, D. D., Gupta, A. and S. Gottesman. The small RNA, dsrA, is essential for the low temperature expression of rpoS during exponential growth in Escherichia coli. EMBO J. 15:3993-4000, 1996.

Smeltzer, M. S., Hart, M. E. and J. J. Iandolo. Phenotypic characterization of xpr, a global regulator of extracellular virulence factors in Staphylococus aureus. Infect. Immun. 61:919-925, 1993.

Smibert, R. M. and N. R. Krieg. General Characterization, p. 435. In Kreig, N. (ed.), Manual of Methods for General Bacteriology. American Society for Microbiology, Washington, D.C., 1981.

Tegmark, K., Karlsson, A. and S. Arvidson. Identification and characterization of SarH1, a new global regulator of virulence gene expression in Staphylococcus aureus. Molec. Microbiol. 37:398-409, 2000.

Tegmark, K., Morfeldt, E. and S. Arvidson. The virulence gene regulator, SarA appears to be a non-specific DNA binding protein. Molec. Microbiol., in press, 2002.

Thomson N. R., Cox, A., Bycroft, B. W., Stewart, G. S., Williams, P., and G. P. Salmond. The Rap and Hor proteins of Erwinia, Serratia and Yersinia: a novel subgroup in a growing superfamily of proteins regulating diverse physiological processes in bacterial pathogens. Mol. Microbiol. 26:531-544, 1997.

Vandenesch, F., Kornblum, J. and R. P. Novick. A temporal signal, independent of agr, is required for hla but not spa transcription in Staphylococcus aureus. J. Bacteriol. 173: 6313-6320, 1991.

Wu, S., de Lancastre, H. and A. Tomaz. Sigma-B, a putative operon encoding alternate sigma factor of Staphylococcus aureus RNA polymerase: Molecular cloning and DNA sequencing. J. Bacteriol. 178:6036-6942, 1996.

Yarwood, J. M. and P. M. Schlievert. Oxygen and carbon dioxide regulation of toxic shock syndrome toxin 1 production in Staphylococcus aureus MN8. J. Clin. Microbiol. 38:1797-1803, 2000.

Youngman, P. Plasmid vectors for recovering and exploiting Tn917 transposotions in Bacillus and other gram-positive bacteria, p. 79-103, In K. G. Hardy (ed.), Plasmids: a practical approach. IRL Press Limited, Oxford, 1987.

Yu, A. V. and V. V. Petrov. Branched respiratory chain in aerobically grown Staphylococcus aureus oxidation of ethanol by cells and protoplasts. Arch. Microbiol. 153:580-584, 1990.

We claim:

1. An isolated polynucleotide comprising a member selected from the group consisting of:
(a) a polynucleotide of SEQ ID NO:1, SEQ ID NO:3, or SEQ ID NO:5 or a complement of SEQ ID NO:1, SEQ ID NO:3, or SEQ ID NO:5;
(b) a fragment of the polynucleotide of SEQ ID NO:1, SEQ ID NO:3, or SEQ ID NO:5 wherein the fragment comprises at least 20 contiguous nucleotides of SEQ ID NO:1, SEQ ID NO:3, or SEQ ID NO:5;
(c) a polynucleotide having at least 70% sequence identity to the sequence of SEQ ID NO:1, SEQ ID NO:3, or SEQ ID NO:5;
(d) a polynucleotide that hybridizes to any one of the polynucleotides of (a), (b) or (c) under conditions of 5× SSC, 50% formamide and 42° C., and which encodes a protein having the same biological function; and
(e) a polynucleotide encoding the same amino acid sequence as any of the polynucleotides of (a), (b), (c) or (d) but which exhibits regular degeneracy in accordance with the degeneracy of the genetic code.

2. A recombinant polynucleotide comprising a member selected from the group consisting of:
(a) a polynucleotide of SEQ ID NO:1, SEQ ID NO:3, or SEQ ID NO:5 or a complement of SEQ ID NO:1, SEQ ID NO:3, or SEQ ID NO:5;
(b) a fragment of the polynucleotide of SEQ ID NO:1, SEQ ID NO:3, or SEQ ID NO:5 wherein the fragment comprises at least 20 contiguous nucleotides of SEQ ID NO:1, SEQ ID NO:3, or SEQ ID NO:5;
(c) a polynucleotide having at least 70% sequence identity to the sequence of SEQ ID NO:1, SEQ ID NO:3, or SEQ ID NO:5;
(d) a polynucleotide that hybridizes to any of the polynucleotides of (a), (b) or (c) under conditions of 5× SSC, 50% formamide and 42° C., and which encodes a protein having the same biological function; and
(e) a polynucleotide encoding the same amino acid sequence as any of the polynucleotides of (a), (b), (c) or (d) but which exhibits regular degeneracy in accordance with the degeneracy of the genetic code.

3. The polynucleotide of claim 2 wherein the polynucleotide is selected from the group consisting of:
(a) a polynucleotide of SEQ ID NO:3 or a complement of SEQ ID NO:3;
(b) a fragment of the polynucleotide of SEQ ID NO:3 wherein the fragment comprises at least 20 contiguous nucleotides of SEQ ID NO:3;
(c) a polynucleotide having at least 70% sequence identity to the sequence of SEQ ID NO:3;
(d) a polynucleotide that hybridizes to any of the polynucleotides of (a), (b) or (c) under conditions of 5× SSC, 50% formamide and 42° C., and which encodes a protein having the same biological function; and
(e) a polynucleotide encoding the same amino acid sequence as any of the polynucleotides of (a), (b), (c) or (d) but which exhibits regular degeneracy in accordance with the degeneracy of the genetic code.

4. A recombinant vector comprising a polynucleotide of claim 2.

5. The recombinant vector of claim 4 wherein the vector is selected from the group consisting of plasmids, bacteriophages, cosmids, and viruses.

6. The recombinant vector of claim 4 wherein the vector further comprises at least one additional sequence chosen from the group consisting of:
(a) regulatory sequences operatively coupled to the polynucleotide;
(b) selection markers operatively coupled to the polynucleotide;
(c) marker sequences operatively coupled to the polynucleotide;
(d) a purification moiety operatively coupled to the polynucleotide;
(e) a targeting sequence operatively coupled to the polynucleotide; and
(f) a sequence directing expression of a heterologous polypeptide.

7. The recombinant vector of claim 6 wherein the vector is selected from the group consisting of plasmids, bacteriophages, cosmids, and viruses.

8. The recombinant vector of claim 6 wherein the vector comprises a promoter selected from the group consisting of trp, lac, P_{L}, and T7 polymerase operably coupled to the polynucleotide.

9. A host cell comprising the recombinant vector of any of claims 4 to 8.

10. The host cell as set forth in claim 9 wherein said host cell is selected from the group consisting of mammalian cells, plant cells, insect cells, yeast, bacteria, bacteriophage.

11. The host cell as set forth in claim 9 wherein said host cell expresses a protein encoded by said vector.

12. A protein or polypeptide encoded by the polynucleotide selected from the group consisting of:
(a) a polynucleotide of SEQ ID NO:1, SEQ ID NO:3, or SEQ ID NO:5 or a complement of SEQ ID NO:1, SEQ ID NO:3, or SEQ ID NO:5;
(b) a fragment of the polynucleotide of SEQ ID NO:1, SEQ ID NO:3, or SEQ ID NO:5 wherein the fragment comprises at least 20 contiguous nucleotides of SEQ ID NO:1, SEQ ID NO:3, or SEQ ID NO:5;
(c) a polynucleotide having at least 70% sequence identity to the sequence of SEQ ID NO:1, SEQ ID NO:3, or SEQ ID NO:5;
(d) a polynucleotide that hybridizes to any of the polynucleotides of (a), (b) or (c) under conditions of 5× SSC, 50% formamide and 42° C., and which encodes a protein having the same biological function; and
(e) a polynucleotide encoding the same amino acid sequence as any of the polynucleotides of (a), (b), (c) or (d) but which exhibits regular degeneracy in accordance with the degeneracy of the genetic code.

13. An isolated protein or polypeptide comprising the amino acid sequence of SEQ ID NO:2.

14. A recombinant protein or polypeptide comprising the amino acid sequence of SEQ ID NO:2.

15. An isolated protein or polypeptide comprising a Rot polypeptide sequence from Staphylococcus aureus, wherein the polypeptide comprises at least 15 contiguous amino acids of SEQ ID NO:2.

16. A protein or polypeptide of claim 12 wherein one or more of the amino acids have been substituted with a conserved amino acid and the biological function of the protein has been maintained.

17. A protein or polypeptide of claim 12 wherein the protein or polynucleotide is selected from the group consisting of:
(a) a polynucleotide of SEQ ID NO:3 or a complement of SEQ ID NO:3;
(b) a fragment of the polynucleotide of SEQ ID NO:3 wherein the fragment comprises at least 20 contiguous nucleotides of SEQ ID NO:3;
(c) a polynucleotide having at least 70% sequence identity to the sequence of SEQ ID NO:3;
(d) a polynucleotide that hybridizes to any of the polynucleotides of (a), (b) or (c) under conditions of 5× SSC, 50% formamide and 42° C., and which encodes a protein having the same biological function; and
(e) a polynucleotide encoding the same amino acid sequence as any of the polynucleotides of (a), (b), (c) or (d) but which exhibits regular degeneracy in accordance with the degeneracy of the genetic code.

18. An isolated protein or polypeptide comprising the amino acid sequence as depicted in SEQ ID NO:4.

19. A recombinant protein or polypeptide comprising the amino acid sequence as depicted in SEQ ID NO:4.

20. An isolated protein or polypeptide comprising an Rlp polypeptide sequence from Staphylococcus aureus, wherein the protein or polypeptide comprises at least 15 contiguous amino acids of SEQ ID NO:4.

21. The protein or polypeptide of claim 12 or 17 wherein one or more of the amino acids have been substituted with a conserved amino acid and the biological function of the protein has been maintained.

22. An isolated, purified antibody that specifically binds to a polypeptide of claim 12.

23. The antibody of claim 22 wherein the antibody is a monoclonal antibody.

24. An isolated, purified antibody that specifically binds to a polypeptide as set forth in claim 17.

25. The antibody of claim 24 wherein the antibody is a monclonal antibody.

26. A fusion protein comprising a polypeptide or protein of claim 11 linked to a heterologous protein or polypeptide.

27. A nucleic acid probe or primer comprising at least 20 contiguous nucleotides of a polynucleotide of claim 2.

28. A nucleic acid probe or primer comprising at least 20 contiguous nucleotides complementary to a polynucleotide of claim 2.

29. A nucleic acid probe or primer comprising at least 20 contiguous nucleotides of a polynucleotide of claim 3.

30. A nucleic acid probe or primer comprising at least 20 contiguous nucleotides complementary to a polynucleotide of claim 3.

31. A method for detecting a polynucleotide of claim 2 in a microbial isolate, comprising:
(a) extracting the DNA of a microbial isolate; and
(b) probing said DNA with a labeled nucleic acid probe constructed from a polynucleotide of claim 2.

32. A method for detecting a polynucleotide of claim 3 in a microbial isolate, comprising:
(a) extracting the DNA of a microbial isolate; and
(b) probing said DNA with a labeled nucleic acid probe constructed from a polynucleotide of claim 3.

33. A method for detecting a protein or polypeptide of claim 12 in a biological sample, comprising:
(a) combining a biological sample with an antibody of claim 22 or 24 under conditions that permit the formation of a stable antigen-antibody complex; and
(b) detecting any stable complexes formed in step (a).

34. A method for detecting a protein or polypeptide of claim 17 in a biological sample, comprising:
(a) combining a biological sample with an antibody of claim 23 or 25 under conditions that permit the formation of a stable antigen-antibody complex; and
(b) detecting any stable complexes formed in step (a).

35. A method for detecting an anti-Staphylococcus aureus Rot antibody in a biological sample, comprising:
(a) combining a biological sample which potentially contains an anti-Staphylococcus aureus rot antibody with a polypeptide of claim 12 under conditions which permit formation of a stable antigen-antibody complex; and
(b) detecting any stable complexes formed in step (a).

36. A method for detecting an anti-Staphylococcus aureus Rlp antibody in a biological sample, comprising:
(a) combining a biological sample which potentially contains an anti-Staphylococcus aureus rlp antibody with a polypeptide of claim 12 under conditions which permit formation of a stable antigen-antibody complex; and
(b) detecting any stable complexes formed in step (a).

37. A method for purifying an anti-Staphylococcus aureus Rot antibody present in a biological sample, comprising:
(a) combining the biological sample with a polypeptide of claim 12 under conditions which permit formation of a stable antigen-antibody complex;
(b) separating any stable complexes formed in step (a) from said biological sample; and
(c) isolating said antibody from any stable complexes separated from the biological sample in step (b).

38. A method for purifying an anti-Staphylococcus aureus Rlp antibody in a biological sample, comprising:
(a) combining the biological sample with a polypeptide of claim 12 under conditions which permit formation of a stable antigen-antibody complex;
(b) separating any stable complexes formed in step (a) from said biological sample; and
(c) isolating said antibody from any stable complexes separated from the biological sample in step (b).

39. A method of screening for an antibacterial agent, comprising:
(a) contacting a cell expressing a polypeptide encoded by a gene selected from the group consisting of the polynucleotides as set forth in claim 2 with a test compound; and
(b) determining whether the amount or level of activity of said polypeptide is increased.

40. The method of claim 39 wherein said increase or decrease is measured by assaying the protein level of the expressed polynucleotide.

41. The method of claim 39 wherein said increase or decrease is measured by assaying the RNA level of the expressed polynucleotide.

42. A method of screening to identify test substances which induce or repress the expression of genes which are induced or repressed by a protein or polypeptide of claim 12 comprising:
(a) contacting a cell with a test substance; and
(b) monitoring expression of a transcript or its translation product, wherein the transcript specifically hybridizes to a gene selected from a first and a second group, wherein the first group consists of genes known to be transcriptionally upregulated by Rot: adaB, aldH, alsS, arcD, clfB, clpL, coa, ctpA, dhoM, dltB, dltC, dltD, dnax, epiA, fhuA, gltT, gtaB, guaA, guaB, hemn, hit, hld, holb, hsdR, isaB, lepA, lysP, lytH, lytR, lyts, mnha, msrA, mvaKl, nrdD, nrdE, nrdI, pbux, pth, purM, putp, pycA, recQ, rot, rpoc, sdrC, sigb, sodM, spa, srrB, thrb, thrC, xprT, and the second group consists of genes known to be transcriptionally downregulated by Rot: (hlb), adhe, cysK, ddh, ebhA, ebhB, fmhC(eprh), geh, gntK, gntp, hlgB, hlgC, kdpA, kdpC, lytN, mvaK2, mvaS, narG, pmi, prsA, ptsG, ribD, splA, splB, splC, splD, splE, splF, sspC, ureB, ureC, ureD, ureE, uref, ureG, wherein a test substance is identified if it increases expression of a transcript which specifically hybridizes to a gene in the first group or decreases expression of a transcript which specifically hybridizes to a gene in the second group.

43. The method of claim 42 wherein the transcript specifically hybridizes to a gene selected from the first group.

44. The method of claim 42 wherein the transcript specifically hybridizes to a gene selected from the second group.

45. A diagnostic kit for detecting the presence of Staphylococcus aureus in a sample comprising:
(a) a pair of PCR primers, one member of the pair being a primer of claim 27 and the other being a primer of claim 28;
(b) a polymerase; and
(c) buffers and reagents for use in PCR.

46. A diagnostic kit for detecting the presence of Staphylococcus aureus in a sample comprising:
(a) a pair of PCR primers, one member of the pair being a primer of claim 29 and the other being a primer of claim 30;
(b) a polymerase; and
(c) buffers and reagents suitable for use in PCR.

47. A diagnostic kit for detecting the presence of a polynucleotide of claim 2 in a microbial isolate or patient sample, comprising:
(a) a pair of PCR primers, one member of the pair being a primer of claim 27 and the other being a primer of claim 28;
(b) a polymerase; and
(c) buffers and reagents for use in PCR.

48. A diagnostic kit for detecting the presence of a polynucleotide of claim 3 in a microbial isolate or patient sample, comprising:
(a) a pair of PCR primers, one member of the pair being a primer of claim 29 and the other being a primer of claim 30;
(b) a polymerase; and
(c) buffers and reagents for use in PCR.

49. A diagnostic kit for detecting the presence of a polynucleotide of claim 2 in a microbial isolate or patient sample, comprising:
(a) an antibody of claim 22 or 24; and
(b) one or more ancillary reagents for detecting the presence of a complex between said antibody and a polynucleotide.

50. A diagnostic kit for detecting the presence of the polynucleotide as set forth in claim 3 in a microbial isolate or patient sample, comprising:
(a) an antibody of claim 23 or 25; and
(b) one or more ancillary reagents for detecting the presence of a complex between said antibody and a polynucleotide.

51. A recombinant host cell comprising at least one copy of a recombinant vector of claim 4 wherein said host cell or an ancestor of said host cell was transformed with said recombinant vector to produce said recombinant host cell, and wherein said nucleotide sequence is operably associated with an expression control sequence functional in said recombinant host cell.
